SEQUENCE LISTING (3 sequences)

SEQ ID NO: 1 - protein, 156 aa, Homo sapiens
MEPAAGSSMEPSADWLATAAARGRVEEVRALLEAGALPNAPNSYGRRPIQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPD

SEQ ID NO: 2 - protein, 410 aa, Homo sapiens
MPRERRERDAKERDTMKEDGGAEFSARSRKRKANVTVFLQDPDEEMAKIDRTARDQCGSQPWDNNAVCADPCSLIPTPDKEDDDRVYPNSTCKPRIIAPSRGSPLPVLSWANREEVWKIMLNKEKTYLRDQHFLEQHPLLQPKMRAILLDWLMEVCEVYKLHRETFYLAQDFFDRYMATQENVVKTLLQLIGISSLFIAAKLEEIYPPKLHQFAYVTDGACSGDEILTMELMIMKALKWRLSPLTIVSWLNVYMQVAYLNDLHEVLLPQYPQQIFIQIAELLDLCVLDVDCLEFPYGILAASALYHFSSSELMQKVSGYQWCDIENCVKWMVPFAMVIRETGSSKLKHFRGVADEDAHNIQTHRDSLDLLDKARAKKAMLSEQNRASPLPSGLLTPPQSGKKQSSGPEMA

SEQ ID NO: 3 - protein, 928 aa, Homo sapiens
MPPKTPRKTAATAAAAAAEPPAPPPPPPPEEDPEQDSGPEDLPLVRLEFEETEEPDFTALCQKLKIPDHVRERAWLTWEKVSSVDGVLGGYIQKKKELWGICIFIAAVDLDEMSFTFTELQKNIEISVHKFFNLLKEIDTSTKVDNAMSRLLKKYDVLFALFSKLERTCELIYLTQPSSSISTEINSALVLKVSWITFLLAKGEVLQMEDDLVISFQLMLCVLDYFIKLSPPMLLKEPYKTAVIPINGSPRTPRRGQNRSARIAKQLENDTRIIEVLCKEHECNIDEVKNVYFKNFIPFMNSLGLVTSNGLPEVENLSKRYEEIYLKNKDLDARLFLDHDKTLQTDSIDSFETQRTPRKSNLDEEVNVIPPHTPVRTVMNTIQQLMMILNSASDQPSENLISYFNNCTVNPKESILKRVKDIGYIFKEKFAKAVGQGCVEIGSQRYKLGVRLYYRVMESMLKSEEERLSIQNFSKLLNDNIFHMSLLACALEVVMATYSRSTSQNLDSGTDLSFPWILNVLNLKAFDFYKVIESFIKAEGNLTREMIKHLERCEHRIMESLAWLSDSPLFDLIKQSKDREGPTDHLESACPLNLPLQNNHTAADMYLSPVRSPKKKGSTTRVNSTANAETQATSAFQTQKPLKSTSLSLFYKKVYRLAYLRLNTLCERLLSEHPELEHIIWTLFQHTLQNEYELMRDRHLDQIMMCSMYGICKVKNIDLKFKIIVTAYKDLPHAVQETFKRVLIKEEEYDSIIVFYNSVFMQRLKTNILQYASTRPPTLSPIPHIPRSPYKFPSSPLRIPGGNIYISPLKSPYKISEGLPTPTKMTPRSRILVSIGESFGTSEKFQKINQMVCNSDRVLKRSAEGSNPPKPLKKLRFDIEGSDEADGSKHLPGESKFQQKLAEMTSTRTRMQKQKMNDSMDTSNKEEK

(19) United States
(12) Patent Application Publication
(10) Pub. No.: US 2023/0279004 A1
(43) Pub. Date: Sep. 7, 2023

(54) SOLID FORMS, SALTS, AND PROCESSES OF PREPARATION OF A CDK2 INHIBITOR

(71) Applicant: Incyte Corporation, Wilmington, DE (US)

(72) Inventors: Joseph A. Sclafani, Malvern, PA (US); Daniel Carper, Chadds Ford, PA (US); Zhongjiang Jia, Kennett Square, PA (US); Eric Shi, Wilmington, DE (US); Aibin Zhang, Chester Springs, PA (US); Huaping Zhang, Newark, DE (US); Wenxing Guo, Wilmington, DE (US)

(21) Appl. No.: 18/179,838

(22) Filed: Mar. 7, 2023

Related U.S. Application Data
(60) Provisional application No. 63/317,308, filed on Mar. 7, 2022.

(51) Int. Cl. C07D 471/04

(52) U.S. Cl. CPC C07D 471/04; C07B 2200/13

(57) ABSTRACT

The present application provides solid forms and salts of a compound of Formula (I):

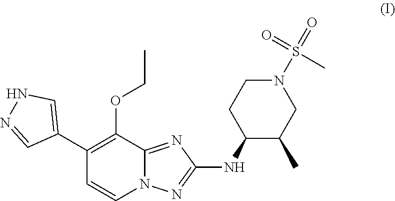 [SMILES: CCOc1c(-c2cn[nH]c2)ccn2nc(N[C@H]3CCN(S(C)(=O)=O)C[C@H]3C)nc12]

pharmaceutical compositions thereof, methods of treating a disease or disorder associated with CDK2 using the same, and processes of preparing the compound of Formula (I) and the solid forms and salts.

This application claims the benefit of priority of U.S. Prov. Appln. No. 63/317,308, filed Mar. 7, 2022, which is incorporated by reference in its entirety.

### SEQUENCE LISTING

This application contains a Sequence Listing that has been submitted electronically as an XML file named 20443-0746001_SL_ST26.xml. The XML file, created on Mar. 6, 2023, is 5,121 bytes in size. The material in the XML file is hereby incorporated by reference in its entirety.

### TECHNICAL FIELD

This application is directed to solid forms and salts of a CDK2 inhibitor, pharmaceutical compositions thereof, methods of treating a disease or disorder associated with CDK2 using the same, and processes of preparing the compound of Formula (I) and the solid forms and salts.

### BACKGROUND

Cyclin-dependent kinases (CDKs) are a family of serine/threonine kinases. Heterodimerized with regulatory subunits known as cyclins, CDKs become fully activated and regulate key cellular processes including cell cycle progression and cell division (Morgan, D. O., Annu Rev Cell Dev Biol, 1997. 13: 261-91). Uncontrolled proliferation is a hallmark of cancer cells. The deregulation of the CDK activity is associated with abnormal regulation of cell-cycle, and is detected in virtually all forms of human cancers (Sherr, C. J., Science, 1996. 274(5293): 1672-7).

CDK2 is of particular interest because deregulation of CDK2 activity occurs frequently in a variety of human cancers. CDK2 plays a crucial role in promoting G1/S transition and S phase progression. In complex with cyclin E (CCNE), CDK2 phosphorylates retinoblastoma pocket protein family members (p107, p130, pRb), leading to de-repression of E2F transcription factors, expression of G1/S transition related genes and transition from G1 to S phase (Henley, S. A. and F. A. Dick, Cell Div, 2012, 7(1): p. 10). This in turn enables activation of CDK2/cyclin A, which phosphorylates endogenous substrates that permit DNA synthesis, replication and centrosome duplication (Ekholm, S. V. and S. I. Reed, Curr Opin Cell Biol, 2000. 12(6): 676-84). It has been reported that the CDK2 pathway influences tumorigenesis mainly through amplification and/or overexpression of CCNE1 and mutations that inactivate CDK2 endogenous inhibitors (e.g., p27), respectively (Xu, X., et al., Biochemistry, 1999. 38(27): 8713-22).

CCNE1 copy-number gain and overexpression have been identified in ovarian, gastric, endometrial, breast and other cancers and been associated with poor outcomes in these tumors (Keyomarsi, K., et al., N Engl J Med, 2002. 347(20): 1566-75; Nakayama, N., et al., Cancer, 2010. 116(11): 2621-34; Au-Yeung, G., et al., Clin Cancer Res, 2017. 23(7): 1862-1874; Rosen, D. G., et al., Cancer, 2006. 106(9): 1925-32). Amplification and/or overexpression of CCNE1 also reportedly contribute to trastuzumab resistance in HER2+ breast cancer and resistance to CDK4/6 inhibitors in estrogen receptor-positive breast cancer (Scaltriti, M., et al., Proc Natl Acad Sci USA, 2011. 108(9): 3761-6; Herrera-Abreu, M. T., et al., Cancer Res, 2016. 76(8): 2301-13). Various approaches targeting CDK2 have been shown to induce cell cycle arrest and tumor growth inhibition (Chen, Y N., et al., Proc Natl Acad Sci USA, 1999. 96(8): 4325-9; Mendoza, N., et al., Cancer Res, 2003. 63(5): 1020-4). Inhibition of CDK2 also reportedly restores sensitivity to trastuzumab treatment in resistant HER2+ breast tumors in a preclinical model (Scaltriti, supra).

These data provide a rationale for considering CDK2 as a potential target for new drug development in cancer associated with deregulated CDK2 activity. In the last decade there has been increasing interest in the development of CDK selective inhibitors. Despite significant efforts, there are no approved agents targeting CDK2 to date (Cicenas, J., et al., Cancers (Basel), 2014. 6(4): p. 2224-42). Therefore it remains a need to discover new forms of CDK2 inhibitors and processes of preparing such inhibitors and solid forms. This application is directed to this need and others.

### SUMMARY

The present disclosure relates to, inter alia, a solid form of a compound of Formula (I):

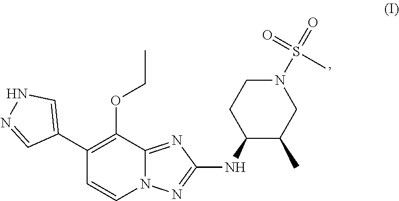 [SMILES: CCOc1c(-c2cn[nH]c2)ccn2nc(N[C@H]3CCN(S(C)(=O)=O)C[C@H]3C)nc12]

which is Form I, Form II, or Form III.

The present disclosure further provides a salt of the compound of Formula (I), which is selected from:
a mono-maleate salt of the compound of Formula (I);
a di-besylate salt of the compound of Formula (I);
a mono-mesylate salt of the compound of Formula (I);
a di-tosylate salt of the compound of Formula (I);
a mono-hydrochloride salt of the compound of Formula (I); and
a di-hydrochloride salt of the compound of Formula (I).

The present disclosure further provides pharmaceutical compositions comprising a solid form of the compound of Formula (I) as described herein, and a pharmaceutically acceptable carrier. The present disclosure also provides pharmaceutical compositions comprising a salt of the compound of Formula (I) as described herein, and a pharmaceutically acceptable carrier.

The present disclosure further provides methods of inhibiting CDK2, comprising contacting the CDK2 with a solid form of Formula (I) as described herein. The present disclosure further provides methods of inhibiting CDK2, comprising contacting the CDK2 with a salt of the compound of Formula (I) as described herein.

The present disclosure further provides methods of inhibiting CDK2 in a patient, comprising administering to the patient a solid form of the compound of Formula (I) as described herein. The present disclosure further provides methods of inhibiting CDK2 in a patient, comprising administering to the patient a salt of the compound of Formula (I) as described herein.

The present disclosure further provides methods of treating a disease or disorder associated with CDK2 in a patient, comprising administering to the patient a solid form of the compound of Formula (I) as described herein. The present disclosure further provides methods of treating a disease or disorder associated with CDK2 in a patient, comprising administering to the patient a salt of the compound of Formula (I) as described herein.

The present disclosure further provides a solid form of the compound of Formula (I) as described herein for use in any of the methods described herein. The present disclosure further provides a salt of the compound of Formula (I) as described herein for use in any of the methods described herein.

The present disclosure further provides uses of a solid form of the compound of Formula (I) as described herein for the preparation of a medicament for use in any of the methods described herein. The present disclosure further provides uses of a salt of the compound of Formula (I) as described herein for the preparation of a medicament for use in any of the methods described herein.

The present disclosure further provides processes of preparing a solid form of the compound of Formula (I) as described herein, comprising cooling a solution of the compound of Formula (I) in a solvent component comprising ethanol and water.

The present disclosure also provides processes of preparing the salts of the compound of Formula (I) as described herein.

The present disclosure further provides processes of preparing a compound of Formula (I) as described herein, or a pharmaceutically acceptable salt thereof, a solid form of the compound of Formula (I) as described herein, or a salt of the compound of Formula (I) as described herein, the process comprising:
reacting a compound of Formula (1c):

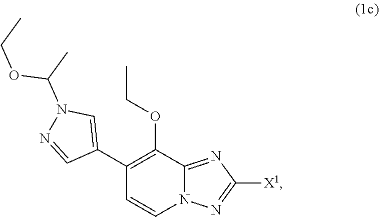

with a compound of Formula (1b):

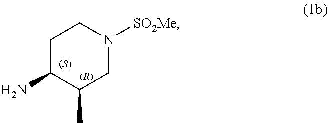

or a salt thereof, via a Buchwald coupling reaction, to form a compound of Formula (1a):

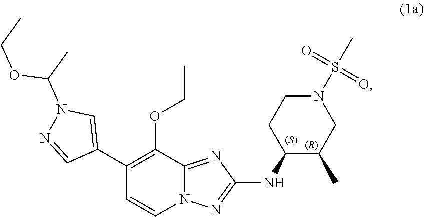

wherein X¹ is halo.

### DESCRIPTION OF DRAWINGS

FIG. 1 shows an XRPD pattern for Form I of crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) free base.

FIG. 2 shows a DSC thermogram for Form I of crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) free base.

FIG. 3 shows a TGA thermogram for Form I of crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) free base.

FIG. 4 shows an XRPD pattern for crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) maleate salt.

FIG. 5 shows a DSC thermogram for crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) maleate salt.

FIG. 6 shows a TGA thermogram for crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) maleate salt.

FIG. 7 shows an XRPD pattern for crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) besylate salt.

FIG. 8 shows a DSC thermogram for crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) besylate salt.

FIG. 9 shows a TGA thermogram for crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) besylate salt.

FIG. 10 shows an XRPD pattern for crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) mesylate salt.

FIG. 11 shows a DSC thermogram for crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) mesylate salt.

FIG. 12 shows a TGA thermogram for crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) mesylate salt.

FIG. 13 shows an XRPD pattern for crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) tosylate salt.

FIG. 14 shows a DSC thermogram for crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) tosylate salt.

FIG. 15 shows a TGA thermogram for crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) tosylate salt.

FIG. 16 shows an XRPD pattern for crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) mono-hydrochloride salt.

FIG. 17 shows a DSC thermogram for crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) mono-hydrochloride salt.

FIG. 18 shows a TGA thermogram for crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) mono-hydrochloride salt.

FIG. 19 shows an XRPD pattern for crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) di-hydrochloride salt.

FIG. 20 shows a DSC thermogram for crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) di-hydrochloride salt.

FIG. 21 shows a TGA thermogram for crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) di-hydrochloride salt.

FIG. 22 shows an XRPD pattern for Form II of crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) free base.

FIG. 23 shows a DSC thermogram for Form II of crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) free base.

FIG. 24 shows a TGA thermogram for Form II of crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) free base.

FIG. 25 shows an XRPD pattern for Form III of crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) free base.

FIG. 26 shows a DSC thermogram for Form III of crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) free base.

FIG. 27 shows a TGA thermogram for Form III of crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) free base.

FIG. 28 shows an XRPD pattern for Form I of crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) free base.

FIG. 29 shows a DSC thermogram for Form I of crystalline 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I)) free base.

### DETAILED DESCRIPTION

### Solid Form and Salts

The present application provides, inter alia, a solid form of a compound of Formula (I):

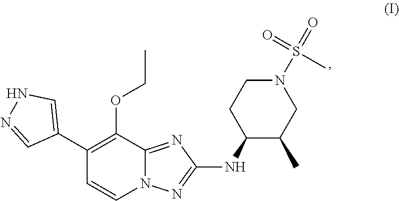 [SMILES: CCOc1c(-c2cn[nH]c2)ccn2nc(N[C@H]3CCN(S(C)(=O)=O)C[C@H]3C)nc12]

which is Form I. Form I is the free base of the compound of Formula (I). In some embodiments, the solid form is non-solvated. In some embodiments, the solid form is crystalline.

In some embodiments, the solid form has at least one XRPD peak, in terms of 2-theta (±0.2 degrees), selected from 7.3, 10.5, 12.8, 14.5, 15.2, 16.4, 20.3, 21.3, 21.6, and 27.0.

In some embodiments, the solid form has at least two XRPD peaks, in terms of 2-theta (±0.2 degrees), selected from 7.3, 10.5, 12.8, 14.5, 15.2, 16.4, 20.3, 21.3, 21.6, and 27.0.

In some embodiments, the solid form has at least three XRPD peaks, in terms of 2-theta (±0.2 degrees), selected from 7.3, 10.5, 12.8, 14.5, 15.2, 16.4, 20.3, 21.3, 21.6, and 27.0.

In some embodiments, the solid form has at least four XRPD peaks, in terms of 2-theta (±0.2 degrees), selected from 7.3, 10.5, 12.8, 14.5, 15.2, 16.4, 20.3, 21.3, 21.6, and 27.0.

In some embodiments, the solid form has at least five XRPD peaks, in terms of 2-theta (±0.2 degrees), selected from 7.3, 10.5, 12.8, 14.5, 15.2, 16.4, 20.3, 21.3, 21.6, and 27.0.

In some embodiments, the solid form has at least ten XRPD peaks, in terms of 2-theta (±0.2 degrees), selected from 7.3, 10.5, 12.8, 14.5, 15.2, 16.4, 20.3, 21.3, 21.6, and 27.0.

In some embodiments, the solid form has at least one XRPD peak, in terms of 2-theta (±0.2 degrees), selected from 7.5, 13.0, 14.7, 15.3, 16.2, 16.6, 20.5, 20.8, 21.4, 23.3, 24.0, and 27.1.

In some embodiments, the solid form has at least two XRPD peaks, in terms of 2-theta (±0.2 degrees), selected from 7.5, 13.0, 14.7, 15.3, 16.2, 16.6, 20.5, 20.8, 21.4, 23.3, 24.0, and 27.1.

In some embodiments, the solid form has at least three XRPD peaks, in terms of 2-theta (±0.2 degrees), selected from 7.5, 13.0, 14.7, 15.3, 16.2, 16.6, 20.5, 20.8, 21.4, 23.3, 24.0, and 27.1.

In some embodiments, the solid form has at least four XRPD peaks, in terms of 2-theta (±0.2 degrees), selected from 7.5, 13.0, 14.7, 15.3, 16.2, 16.6, 20.5, 20.8, 21.4, 23.3, 24.0, and 27.1.

In some embodiments, the solid form has at least five XRPD peaks, in terms of 2-theta (±0.2 degrees), selected from 7.5, 13.0, 14.7, 15.3, 16.2, 16.6, 20.5, 20.8, 21.4, 23.3, 24.0, and 27.1.

In some embodiments, the solid form has at least ten XRPD peaks, in terms of 2-theta (±0.2 degrees), selected from 7.5, 13.0, 14.7, 15.3, 16.2, 16.6, 20.5, 20.8, 21.4, 23.3, 24.0, and 27.1.

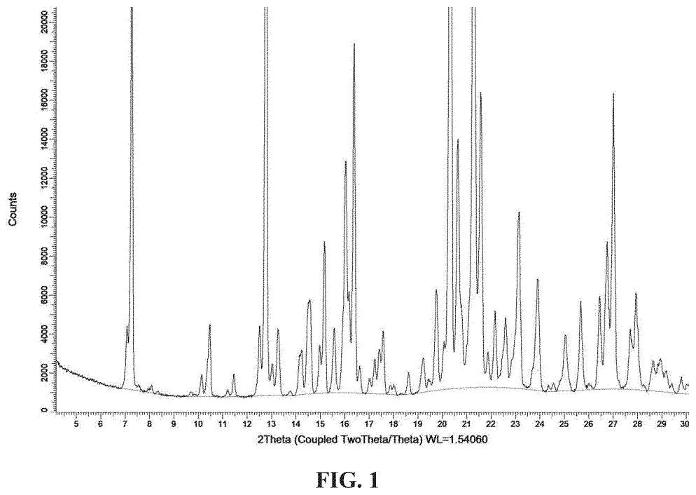

In some embodiments, the solid form has an XRPD pattern as substantially shown in FIG. 1.

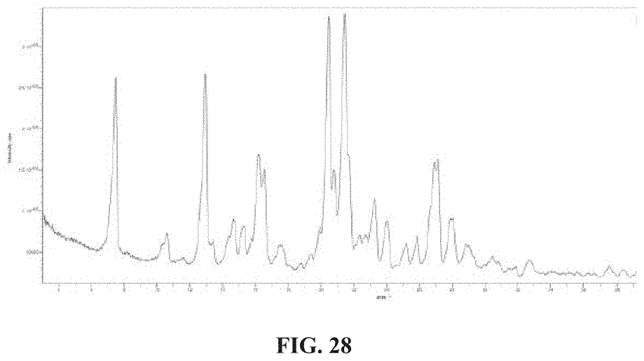

In some embodiments, the solid form has an XRPD pattern as substantially shown in FIG. 28.

In some embodiments, the solid form has an endothermic peak with an onset temperature (±3° C.) at 191.7° C. and a maximum at 193.6° C.

In some embodiments, the solid form has an endothermic peak with an onset temperature (±3° C.) at 191.3° C. and a maximum at 193.3° C.

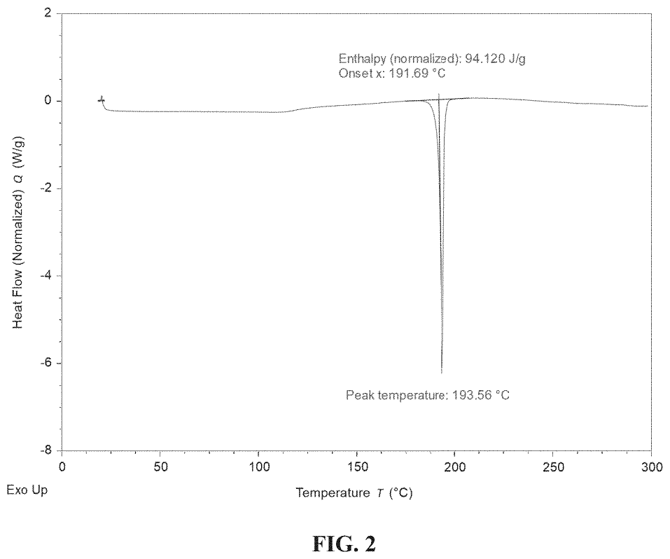

In some embodiments, the solid form has a DSC thermogram substantially as shown in FIG. 2.

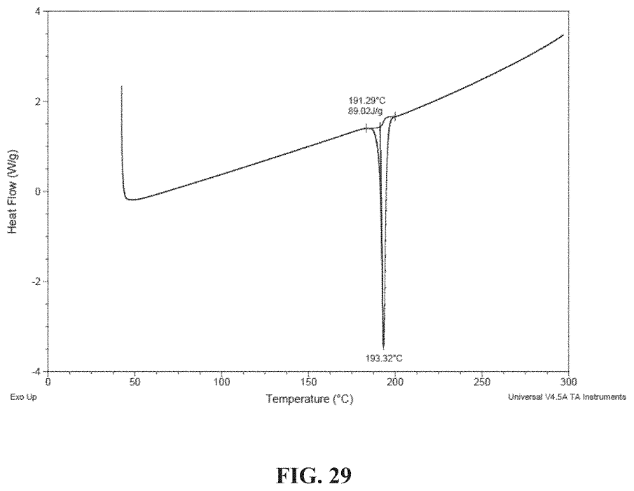

In some embodiments, the solid form has a DSC thermogram substantially as shown in FIG. 29.

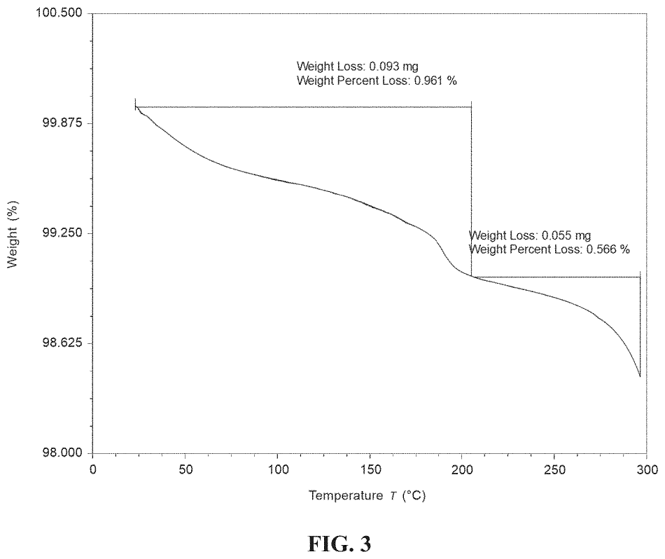

In some embodiments, the solid form has a TGA thermogram substantially as shown in FIG. 3.

The present application also provides a solid form of a compound of Formula (I), which is Form II. Form II is the free base of the compound of Formula (I). In some embodiments, the solid form is non-solvated. In some embodiments, the solid form is crystalline.

In some embodiments, the solid form has at least one XRPD peak, in terms of 2-theta (0.2 degrees), selected from 5.8, 7.6, 11.4, 12.5, 14.4, 17.2, 17.9, and 25.3.

In some embodiments, the solid form has at least two XRPD peaks, in terms of 2-theta (0.2 degrees), selected from 5.8, 7.6, 11.4, 12.5, 14.4, 17.2, 17.9, and 25.3.

In some embodiments, the solid form has at least three XRPD peaks, in terms of 2-theta (0.2 degrees), selected from 5.8, 7.6, 11.4, 12.5, 14.4, 17.2, 17.9, and 25.3.

In some embodiments, the solid form has at least four XRPD peaks, in terms of 2-theta (0.2 degrees), selected from 5.8, 7.6, 11.4, 12.5, 14.4, 17.2, 17.9, and 25.3.

In some embodiments, the solid form has at least five XRPD peaks, in terms of 2-theta (0.2 degrees), selected from 5.8, 7.6, 11.4, 12.5, 14.4, 17.2, 17.9, and 25.3.

In some embodiments, the solid form has at least ten XRPD peaks, in terms of 2-theta (0.2 degrees), selected from 5.8, 7.6, 11.4, 12.5, 14.4, 17.2, 17.9, and 25.3.

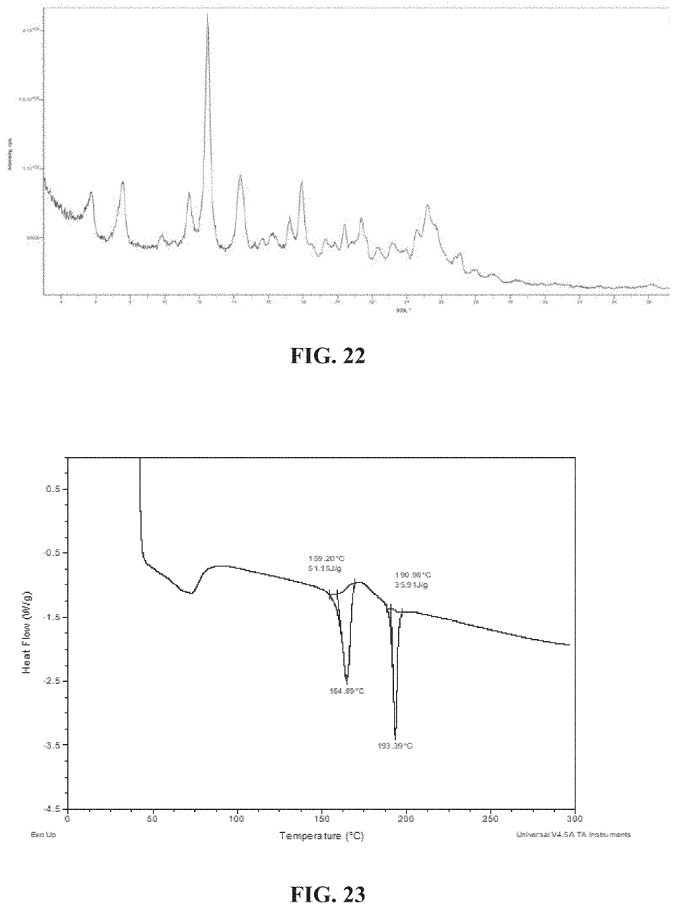

In some embodiments, the solid form has an XRPD pattern as substantially shown in FIG. 22.

In some embodiments, the solid form has an endothermic peak with an onset temperature (±3° C.) at 191.0° C. and a maximum at 193.4° C.

In some embodiments, the solid form has a DSC thermogram substantially as shown in FIG. 23.

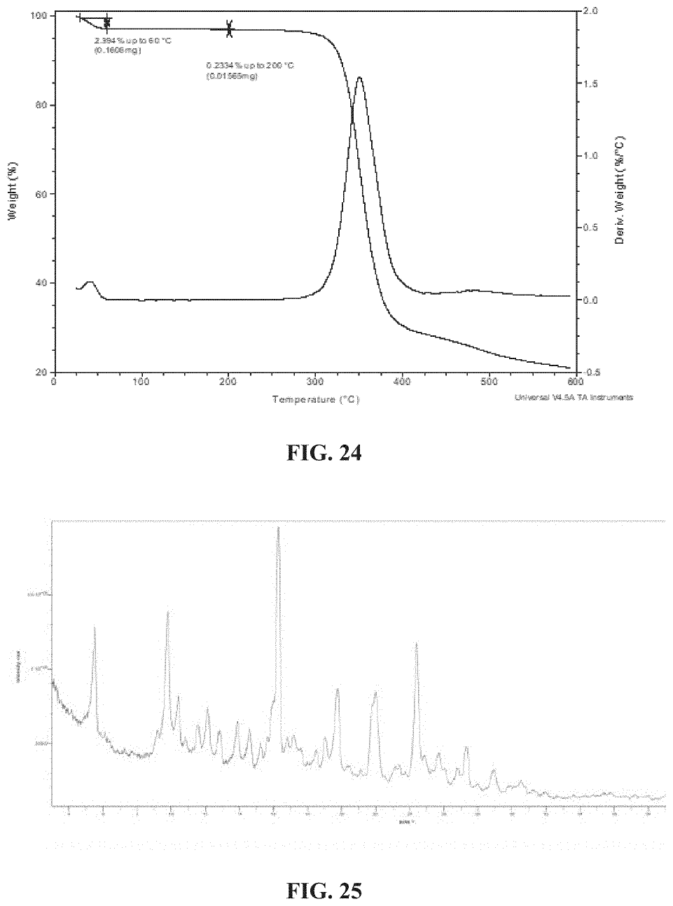

In some embodiments, the solid form has a TGA thermogram substantially as shown in FIG. 24.

The present application also provides a solid form of a compound of Formula (I), which is Form III. Form III is the free base of the compound of Formula (I). In some embodiments, the solid form is solvated. In some embodiments, the solid form is a 1,4-dioxane solvate. In some embodiments, the 1,4-dioxane solvate of the compound of Formula (I) has a stoichiometric ratio of 4:1 of the compound of Formula (I) to 1,4-dioxane. In some embodiments, the solid form is crystalline.

In some embodiments, the solid form has at least one XRPD peak, in terms of 2-theta (0.2 degrees), selected from 5.5, 9.8, 10.5, 12.1, 13.9, 16.3, 19.8, 22.0, 24.4, and 27.3.

In some embodiments, the solid form has at least two XRPD peaks, in terms of 2-theta (0.2 degrees), selected from 5.5, 9.8, 10.5, 12.1, 13.9, 16.3, 19.8, 22.0, 24.4, and 27.3.

In some embodiments, the solid form has at least three XRPD peaks, in terms of 2-theta (0.2 degrees), selected from 5.5, 9.8, 10.5, 12.1, 13.9, 16.3, 19.8, 22.0, 24.4, and 27.3.

In some embodiments, the solid form has at least four XRPD peaks, in terms of 2-theta (0.2 degrees), selected from 5.5, 9.8, 10.5, 12.1, 13.9, 16.3, 19.8, 22.0, 24.4, and 27.3.

In some embodiments, the solid form has at least five XRPD peaks, in terms of 2-theta (0.2 degrees), selected from 5.5, 9.8, 10.5, 12.1, 13.9, 16.3, 19.8, 22.0, 24.4, and 27.3.

In some embodiments, the solid form has at least ten XRPD peaks, in terms of 2-theta (0.2 degrees), selected from 5.5, 9.8, 10.5, 12.1, 13.9, 16.3, 19.8, 22.0, 24.4, and 27.3.

In some embodiments, the solid form has an XRPD pattern as substantially shown in FIG. 25.

In some embodiments, the solid form has an endothermic peak with an onset temperature (±3° C.) at 192.6° C. and a maximum at 194.3° C.

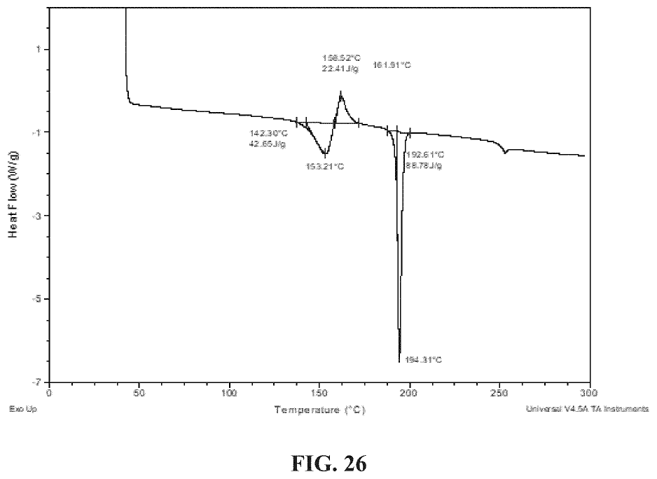

In some embodiments, the solid form has a DSC thermogram substantially as shown in FIG. 26.

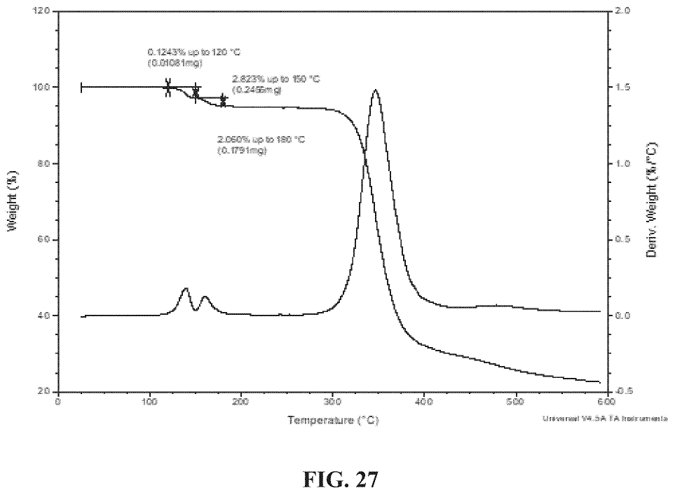

In some embodiments, the solid form has a TGA thermogram substantially as shown in FIG. 27.

Also provided in the present application is a salt of a compound of Formula (I):

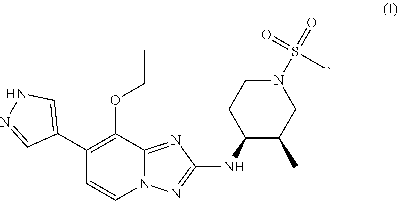 [SMILES: CCOc1c(-c2cn[nH]c2)ccn2nc(N[C@H]3CCN(S(C)(=O)=O)C[C@H]3C)nc12]

which is selected from a mono-maleate salt of the compound of Formula (I); a di-besylate salt of the compound of Formula (I); a mono-mesylate salt of the compound of Formula (I); a di-tosylate salt of the compound of Formula (I); a mono-hydrochloride salt of the compound of Formula (I); and a di-hydrochloride salt of the compound of Formula (I).

In some embodiments, the salt is a mono-maleate salt of the compound of Formula (I). In some embodiments, the mono-maleate salt is crystalline.

In some embodiments, the mono-maleate salt has at least one XRPD peak, in terms of 2-theta (0.2 degrees), selected from 10.4, 11.6, 12.0, 14.1, 15.1, 17.2, 18.1, 19.1, 21.3, 21.9, 22.9, 24.2, and 25.9.

In some embodiments, the mono-maleate salt has at least two XRPD peaks, in terms of 2-theta (0.2 degrees), selected from 10.4, 11.6, 12.0, 14.1, 15.1, 17.2, 18.1, 19.1, 21.3, 21.9, 22.9, 24.2, and 25.9.

In some embodiments, the mono-maleate salt has at least three XRPD peaks, in terms of 2-theta (0.2 degrees), selected from 10.4, 11.6, 12.0, 14.1, 15.1, 17.2, 18.1, 19.1, 21.3, 21.9, 22.9, 24.2, and 25.9.

In some embodiments, the mono-maleate salt at least four XRPD peaks, in terms of 2-theta (0.2 degrees), selected from 10.4, 11.6, 12.0, 14.1, 15.1, 17.2, 18.1, 19.1, 21.3, 21.9, 22.9, 24.2, and 25.9.

In some embodiments, the mono-maleate salt has at least five XRPD peaks, in terms of 2-theta (0.2 degrees), selected from 10.4, 11.6, 12.0, 14.1, 15.1, 17.2, 18.1, 19.1, 21.3, 21.9, 22.9, 24.2, and 25.9.

In some embodiments, the mono-maleate salt has at least ten XRPD peaks, in terms of 2-theta (0.2 degrees), selected from 10.4, 11.6, 12.0, 14.1, 15.1, 17.2, 18.1, 19.1, 21.3, 21.9, 22.9, 24.2, and 25.9.

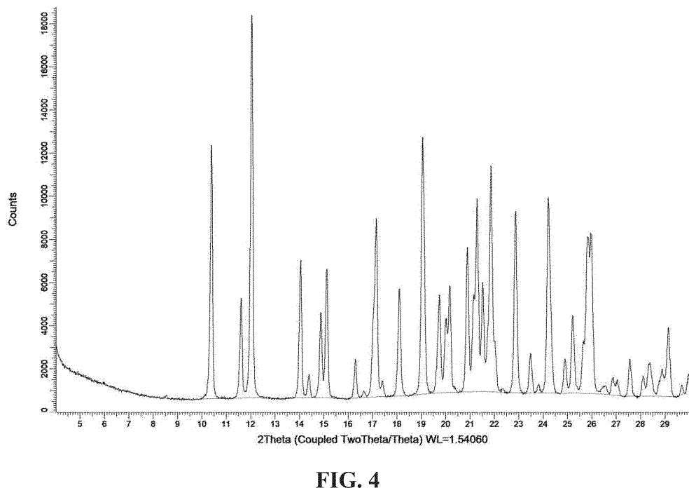

In some embodiments, the mono-maleate salt has an XRPD pattern as substantially shown in FIG. 4.

In some embodiments, the mono-maleate salt has an endothermic peak with an onset temperature (±3° C.) at 180.4° C. and a maximum temperature (±3° C.) at 181.8° C.

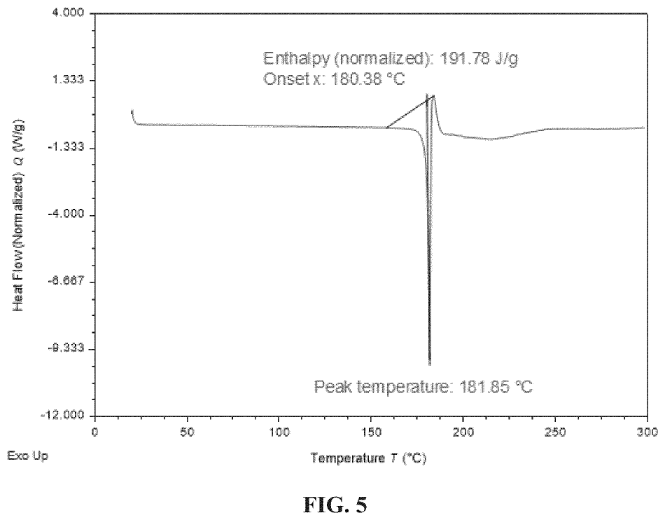

In some embodiments, the mono-maleate salt has a DSC thermogram substantially as shown in FIG. 5.

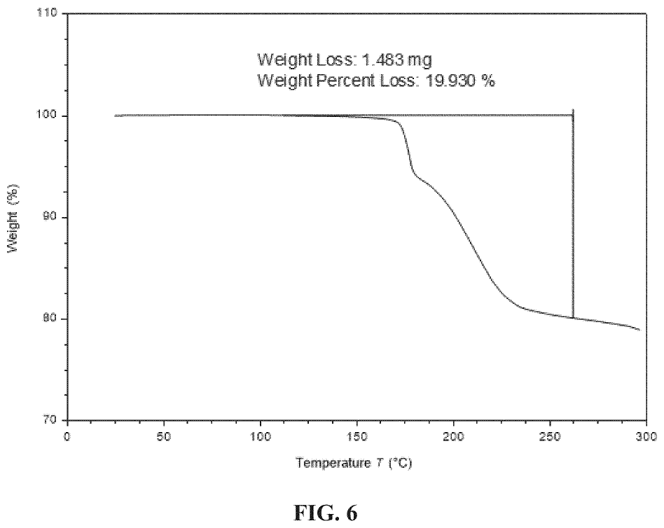

In some embodiments, the mono-maleate salt has a TGA thermogram substantially as shown in FIG. 6.

In some embodiments, the salt is a di-besylate salt of the compound of Formula (I). In some embodiments, the di-besylate salt is crystalline.

In some embodiments, the di-besylate salt has at least one XRPD peak, in terms of 2-theta (±0.2 degrees), selected from 6.3, 9.9, 12.1, 12.6, 15.9, 17.4, 18.7, 19.0, 19.6, and 25.1.

In some embodiments, the di-besylate salt has at least two XRPD peaks, in terms of 2-theta (±0.2 degrees), selected from 6.3, 9.9, 12.1, 12.6, 15.9, 17.4, 18.7, 19.0, 19.6, and 25.1.

In some embodiments, the di-besylate salt has at least three XRPD peaks, in terms of 2-theta (±0.2 degrees), selected from 6.3, 9.9, 12.1, 12.6, 15.9, 17.4, 18.7, 19.0, 19.6, and 25.1.

In some embodiments, the di-besylate salt has at least four XRPD peaks, in terms of 2-theta (±0.2 degrees), selected from 6.3, 9.9, 12.1, 12.6, 15.9, 17.4, 18.7, 19.0, 19.6, and 25.1.

In some embodiments, the di-besylate salt has at least five XRPD peaks, in terms of 2-theta (±0.2 degrees), selected from 6.3, 9.9, 12.1, 12.6, 15.9, 17.4, 18.7, 19.0, 19.6, and 25.1.

In some embodiments, the di-besylate salt has at least ten XRPD peaks, in terms of 2-theta (±0.2 degrees), selected from 6.3, 9.9, 12.1, 12.6, 15.9, 17.4, 18.7, 19.0, 19.6, and 25.1.

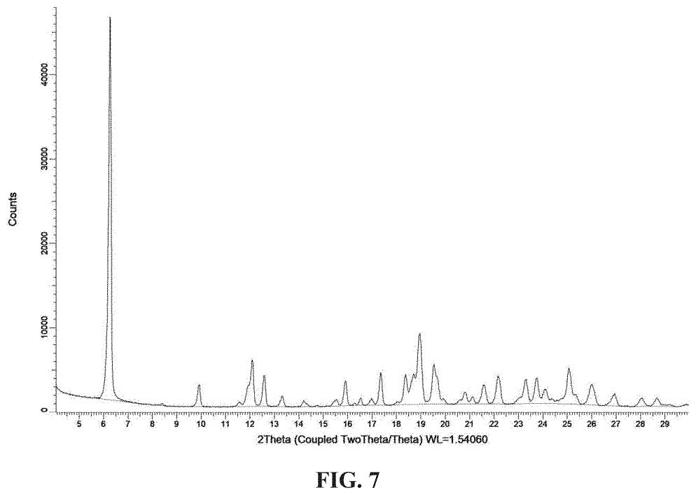

In some embodiments, the di-besylate salt has an XRPD pattern as substantially shown in FIG. 7.

In some embodiments, the di-besylate salt has an endothermic peak with an onset temperature (±3° C.) at 160.4° C. and a maximum temperature (±3° C.) at 163.4° C.

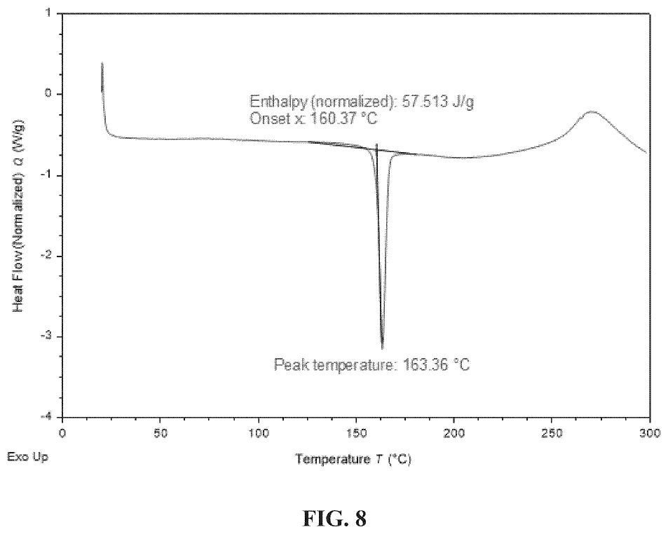

In some embodiments, the di-besylate salt has a DSC thermogram substantially as shown in FIG. 8.

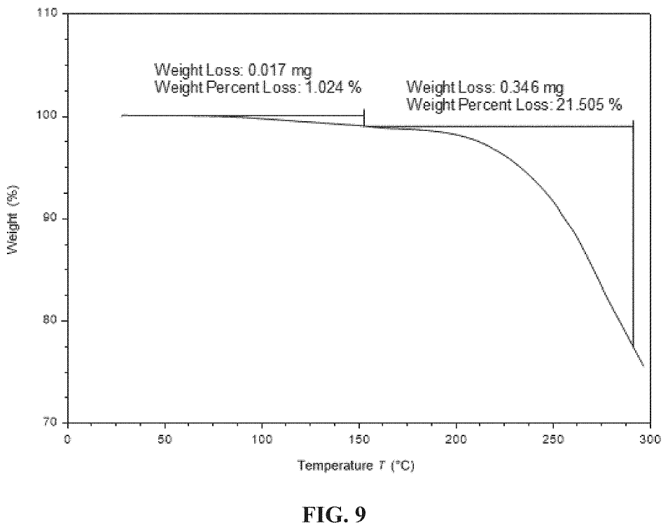

In some embodiments, the di-besylate salt has a TGA thermogram substantially as shown in FIG. 9.

In some embodiments, the salt is a mono-mesylate salt of the compound of Formula (I). In some embodiments, the mono-mesylate salt is crystalline.

In some embodiments, the mono-mesylate salt has at least one XRPD peak, in terms of 2-theta (±0.2 degrees), selected from 4.8, 7.0, 11.9, 14.1, 14.9, 17.7, 18.9, 20.2, 22.1, and 26.1.

In some embodiments, the mono-mesylate salt has at least two XRPD peaks, in terms of 2-theta (±0.2 degrees), selected from 4.8, 7.0, 11.9, 14.1, 14.9, 17.7, 18.9, 20.2, 22.1, and 26.1.

In some embodiments, the mono-mesylate salt has at least three XRPD peaks, in terms of 2-theta (±0.2 degrees), selected from 4.8, 7.0, 11.9, 14.1, 14.9, 17.7, 18.9, 20.2, 22.1, and 26.1.

In some embodiments, the mono-mesylate salt has at least four XRPD peaks, in terms of 2-theta (±0.2 degrees), selected from 4.8, 7.0, 11.9, 14.1, 14.9, 17.7, 18.9, 20.2, 22.1, and 26.1.

In some embodiments, the mono-mesylate salt has at least five XRPD peaks, in terms of 2-theta (±0.2 degrees), selected from 4.8, 7.0, 11.9, 14.1, 14.9, 17.7, 18.9, 20.2, 22.1, and 26.1.

In some embodiments, the mono-mesylate salt has at least ten XRPD peaks, in terms of 2-theta (±0.2 degrees), selected from 4.8, 7.0, 11.9, 14.1, 14.9, 17.7, 18.9, 20.2, 22.1, and 26.1.

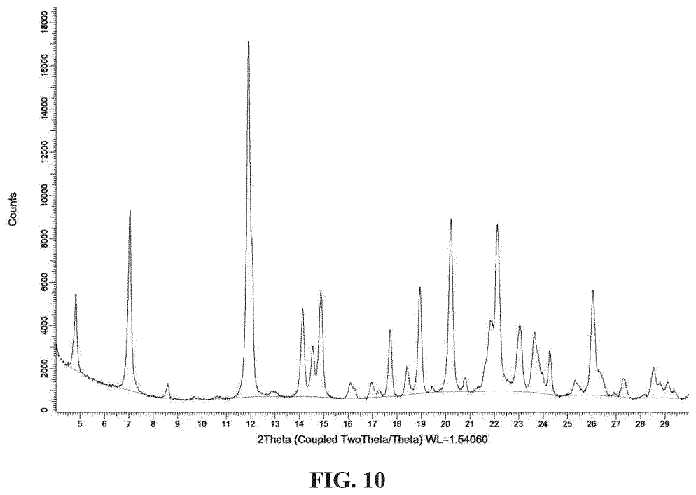

In some embodiments, the mono-mesylate salt has an XRPD pattern as substantially shown in FIG. 10.

In some embodiments, the mono-mesylate salt has a first endothermic peak with a maximum temperature (±3° C.) at 61.1° C. and a second endothermic peak with an onset temperature (±3° C.) at 134.4° C. and a maximum temperature (±3° C.) at 150.1° C.

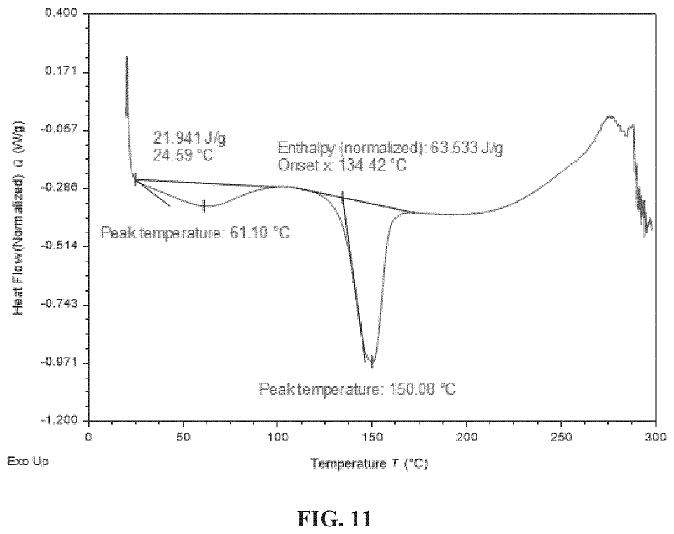

In some embodiments, the mono-mesylate salt has a DSC thermogram substantially as shown in FIG. 11.

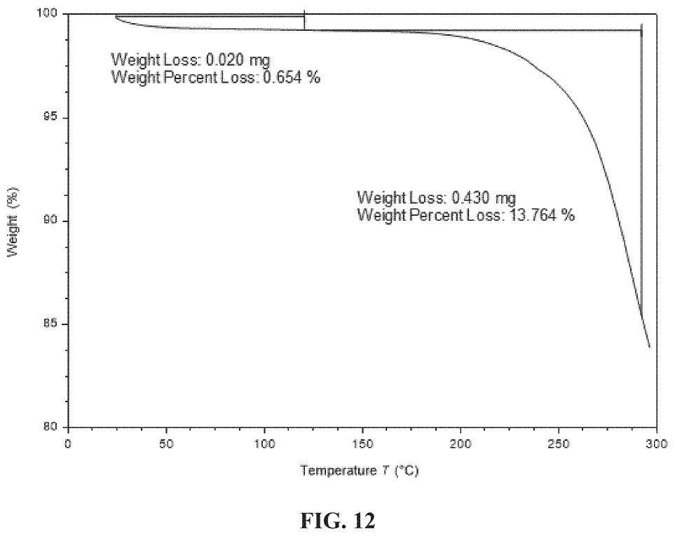

In some embodiments, the mono-mesylate salt has a TGA thermogram substantially as shown in FIG. 12.

In some embodiments, the salt is a di-tosylate salt of the compound of Formula (I). In some embodiments, the di-tosylate salt is crystalline.

In some embodiments, the di-tosylate salt has at least one XRPD peak, in terms of 2-theta (0.2 degrees), selected from 5.7, 7.8, 8.1, 9.3, 13.7, 13.9, 16.2, 18.8, and 20.6.

In some embodiments, the di-tosylate salt has at least two XRPD peaks, in terms of 2-theta (±0.2 degrees), selected from 5.7, 7.8, 8.1, 9.3, 13.7, 13.9, 16.2, 18.8, and 20.6.

In some embodiments, the di-tosylate salt has at least three XRPD peaks, in terms of 2-theta (±0.2 degrees), selected from 65.7, 7.8, 8.1, 9.3, 13.7, 13.9, 16.2, 18.8, and 20.6.

In some embodiments, the di-tosylate salt has at least four XRPD peaks, in terms of 2-theta (±0.2 degrees), selected from 5.7, 7.8, 8.1, 9.3, 13.7, 13.9, 16.2, 18.8, and 20.6.

In some embodiments, the di-tosylate salt has at least five XRPD peaks, in terms of 2-theta (±0.2 degrees), selected from 5.7, 7.8, 8.1, 9.3, 13.7, 13.9, 16.2, 18.8, and 20.6.

In some embodiments, the di-tosylate salt has at least eight XRPD peaks, in terms of 2-theta (±0.2 degrees), selected from 5.7, 7.8, 8.1, 9.3, 13.7, 13.9, 16.2, 18.8, and 20.6.

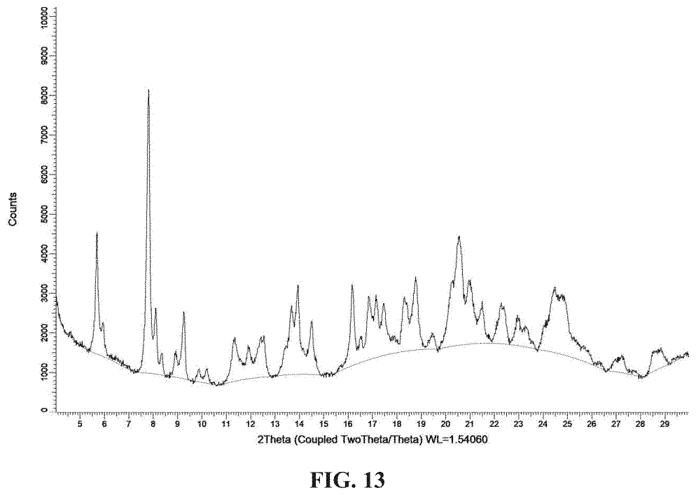

In some embodiments, the di-tosylate salt has an XRPD pattern as substantially shown in FIG. 13.

In some embodiments, the di-tosylate salt has an exothermic peak with an onset temperature (±3° C.) at 99.6° C. and a maximum temperature (±3° C.) at 110.5° C., and an endothermic peak with an onset temperature (±3° C.) at 216.1° C. and a maximum temperature (±3° C.) at 218.7° C.

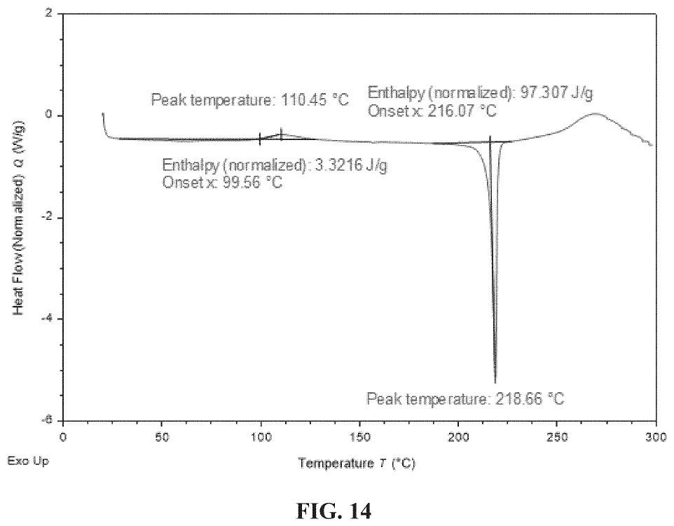

In some embodiments, the di-tosylate salt has a DSC thermogram substantially as shown in FIG. 14.

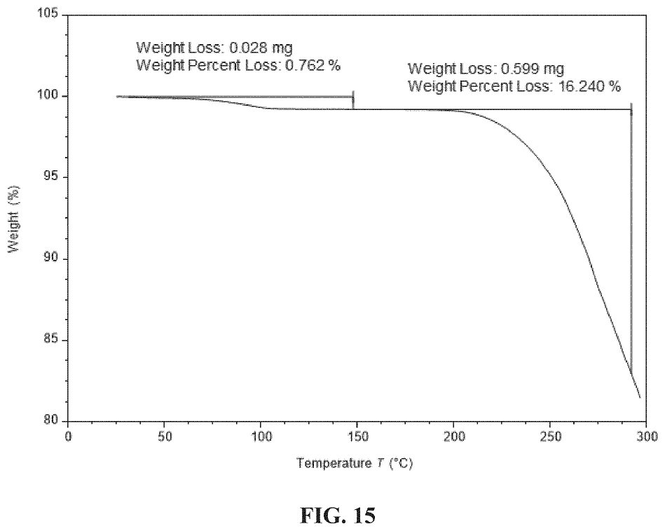

In some embodiments, the di-tosylate salt has a TGA thermogram substantially as shown in FIG. 15.

In some embodiments, the salt is a mono-hydrochloride salt of the compound of Formula (I). In some embodiments, the mono-hydrochloride salt is crystalline.

In some embodiments, the mono-hydrochloride salt has at least one XRPD peak, in terms of 2-theta (±0.2 degrees), selected from 5.7, 8.5, 11.3, 14.1, 15.0, 18.4, 19.3, 20.5, 21.8, 22.8, and 25.7.

In some embodiments, the mono-hydrochloride salt has at least two XRPD peaks, in terms of 2-theta (±0.2 degrees), selected from 5.7, 8.5, 11.3, 14.1, 15.0, 18.4, 19.3, 20.5, 21.8, 22.8, and 25.7.

In some embodiments, the mono-hydrochloride salt has at least three XRPD peaks, in terms of 2-theta (0.2 degrees), selected from 5.7, 8.5, 11.3, 14.1, 15.0, 18.4, 19.3, 20.5, 21.8, 22.8, and 25.7.

In some embodiments, the mono-hydrochloride salt has at least four XRPD peaks, in terms of 2-theta (±0.2 degrees), selected from 5.7, 8.5, 11.3, 14.1, 15.0, 18.4, 19.3, 20.5, 21.8, 22.8, and 25.7.

In some embodiments, the mono-hydrochloride salt has at least five XRPD peaks, in terms of 2-theta (0.2 degrees), selected from 5.7, 8.5, 11.3, 14.1, 15.0, 18.4, 19.3, 20.5, 21.8, 22.8, and 25.7.

In some embodiments, the mono-hydrochloride salt has at least ten XRPD peaks, in terms of 2-theta (±0.2 degrees), selected from 5.7, 8.5, 11.3, 14.1, 15.0, 18.4, 19.3, 20.5, 21.8, 22.8, and 25.7.

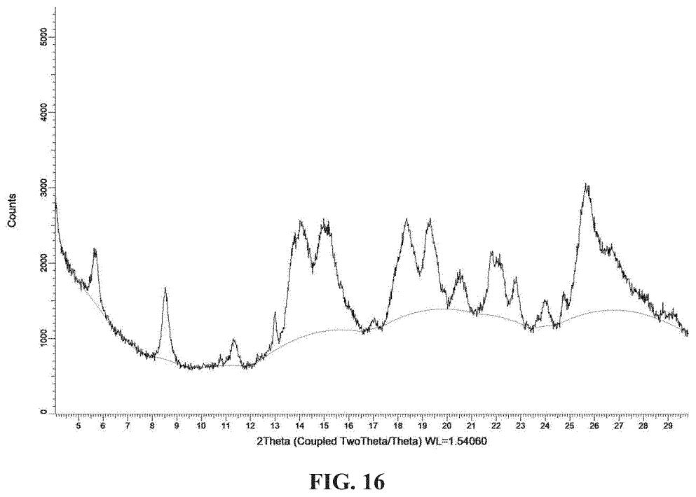

In some embodiments, the mono-hydrochloride salt has an XRPD pattern as substantially shown in FIG. 16.

In some embodiments, the mono-hydrochloride salt has an endothermic peak with an onset temperature (±3° C.) at 196.0° C. and a maximum temperature (±3° C.) at 212.2° C.

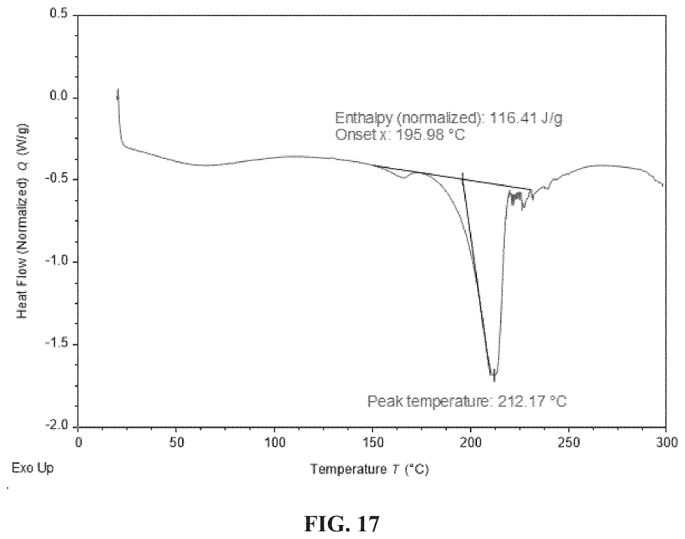

In some embodiments, the mono-hydrochloride salt has a DSC thermogram substantially as shown in FIG. 17.

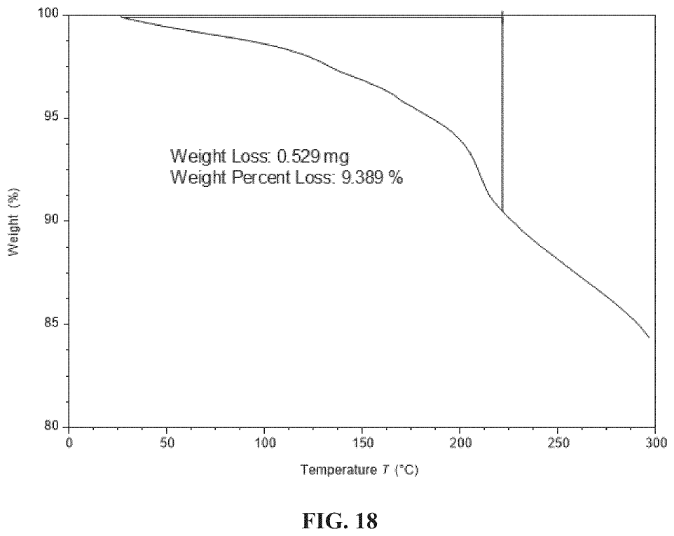

In some embodiments, the mono-hydrochloride salt has a TGA thermogram substantially as shown in FIG. 18.

In some embodiments, the salt is a di-hydrochloride salt of the compound of Formula (I). In some embodiments, the di-hydrochloride salt is crystalline.

In some embodiments, the di-hydrochloride salt has at least one XRPD peak, in terms of 2-theta (±0.2 degrees), selected from 9.9, 10.7, 12.3, 13.0, 14.0, 15.2, 19.9, 21.8, 22.3, and 24.8.

In some embodiments, the di-hydrochloride salt has at least two XRPD peaks, in terms of 2-theta (±0.2 degrees), selected from 9.9, 10.7, 12.3, 13.0, 14.0, 15.2, 19.9, 21.8, 22.3, and 24.8.

In some embodiments, the di-hydrochloride salt has at least three XRPD peaks, in terms of 2-theta (±0.2 degrees), selected from 9.9, 10.7, 12.3, 13.0, 14.0, 15.2, 19.9, 21.8, 22.3, and 24.8.

In some embodiments, the di-hydrochloride salt has at least four XRPD peaks, in terms of 2-theta (±0.2 degrees), selected from 9.9, 10.7, 12.3, 13.0, 14.0, 15.2, 19.9, 21.8, 22.3, and 24.8.

In some embodiments, the di-hydrochloride salt has at least five XRPD peaks, in terms of 2-theta (±0.2 degrees), selected from 9.9, 10.7, 12.3, 13.0, 14.0, 15.2, 19.9, 21.8, 22.3, and 24.8.

In some embodiments, the di-hydrochloride salt has at least ten XRPD peaks, in terms of 2-theta (±0.2 degrees), selected from 9.9, 10.7, 12.3, 13.0, 14.0, 15.2, 19.9, 21.8, 22.3, and 24.8.

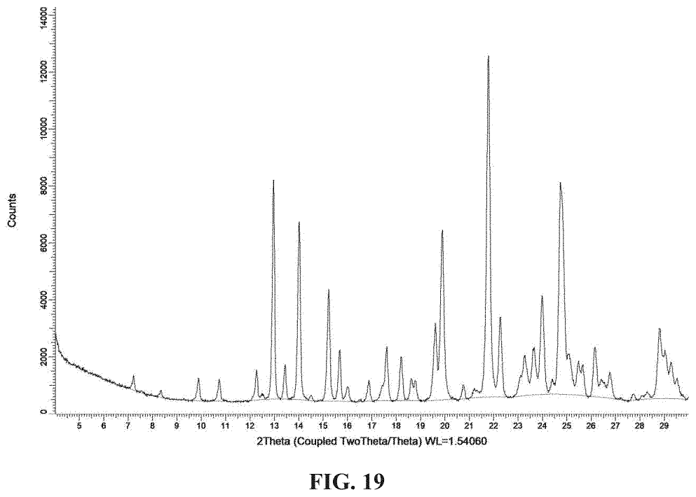

In some embodiments, the di-hydrochloride salt has an XRPD pattern as substantially shown in FIG. 19.

In some embodiments, the di-hydrochloride salt has an endothermic peak with an onset temperature (±3° C.) at 182.1° C. and a maximum temperature (±3° C.) at 206.4° C.

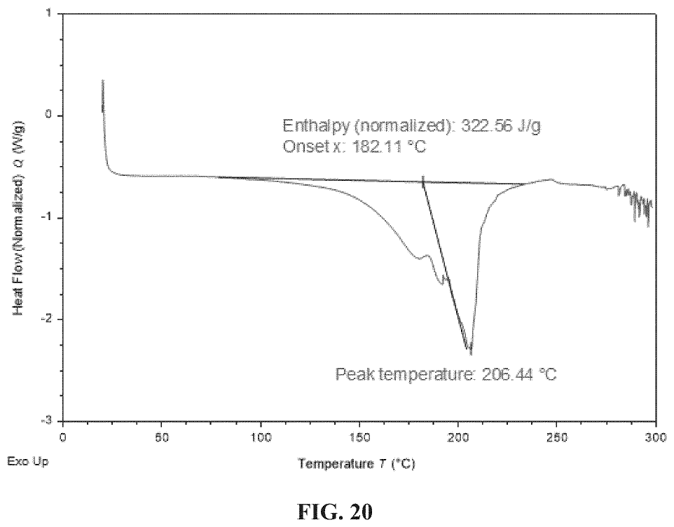

In some embodiments, the di-hydrochloride salt has a DSC thermogram substantially as shown in FIG. 20.

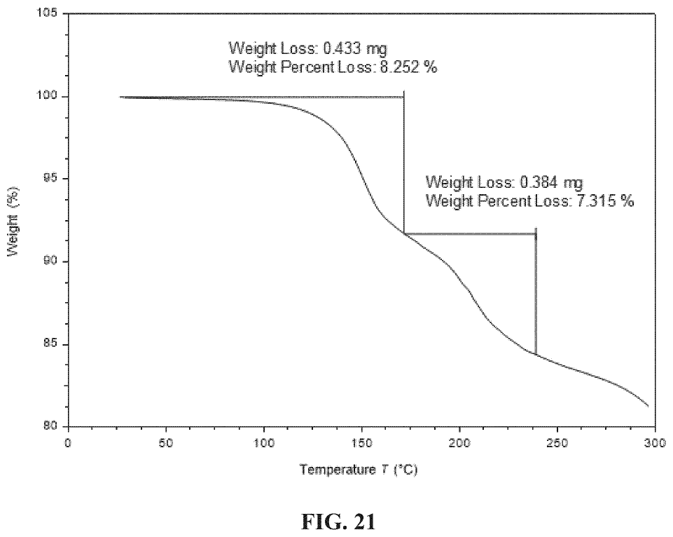

In some embodiments, the di-hydrochloride salt has a TGA thermogram substantially as shown in FIG. 21.

Different forms of the same substance have different bulk properties relating to, for example, hygroscopicity, solubility, stability, and the like. Forms with high melting points often have good thermodynamic stability which is advantageous in prolonging shelf-life drug formulations containing the solid form. Forms with lower melting points often are less thermodynamically stable, but are advantageous in that they have increased water solubility, translating to increased drug bioavailability. Forms that are weakly hygroscopic are desirable for their stability to heat and humidity and are resistant to degradation during long storage.

In some embodiments, the solid form or the salt of the compound of Formula (I) provided herein is crystalline. As used herein, “crystalline” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content.

As used herein, “slurrying” is meant to refer to forming a mixture of insoluble matter in a liquid.

The solid form and salt forms can be identified by solid state characterization methods such as by X-ray powder diffraction (XRPD). Other characterization methods such as differential scanning calorimetry (DSC), thermogravimetric analysis (TGA), dynamic vapor sorption (DVS), solid state NMR, and the like further help identify the form as well as help determine stability and solvent/water content.

An XRPD pattern of reflections (peaks) is typically considered a fingerprint of a particular solid form. It is well known that the relative intensities of the XRPD peaks can widely vary depending on, inter alia, the sample preparation technique, crystal size distribution, various filters used, the sample mounting procedure, and the particular instrument employed. In some instances, new peaks may be observed or existing peaks may disappear, depending on the type of the instrument or the settings. As used herein, the term “peak” refers to a reflection having a relative height/intensity of at least about 4% of the maximum peak height/intensity. Moreover, instrument variation and other factors can affect the 2-theta values. Thus, peak assignments, such as those reported herein, can vary by plus or minus about 0.2° (2-theta), and the term “substantially” and “about” as used in the context of XRPD herein is meant to encompass the above-mentioned variations.

In the same way, temperature readings in connection with DSC, TGA, or other thermal experiments can vary about ±3° C. depending on the instrument, particular settings, sample preparation, etc. Accordingly, a solid form or a salt reported herein having a DSC thermogram “substantially” as shown in any of the Figures or the term “about” is understood to accommodate such variation.

In some embodiments, the solid form or the salts described herein are substantially isolated. By “substantially isolated” is meant that the solid form or the salts is at least partially or substantially separated from the environment in which it was formed or detected. Partial separation can include, for example, a composition enriched in the solid form or the salts described herein. Substantial separation can include compositions containing at least about 50%, at least about 60%, at least about 70%, at least about 80%, at least about 90%, at least about 95%, at least about 97%, or at least about 99% by weight of the solid form or the salts described herein.

### Processes of Preparation

The present application further provides a process for preparing a solid form, which is Form I, comprising cooling a solution of the compound of Formula (I) in a solvent component comprising ethanol and water.

In some embodiments, the solvent component comprises about 5% to about 20% water and about 80% to about 95% ethanol. In some embodiments, the solvent component comprises about 5% to about 10% water and about 90% to about 95% ethanol. In some embodiments, the solvent component comprises about 6% water and about 94% ethanol. In some embodiments, the solvent component comprises about 10% water and about 90% ethanol.

In some embodiments, the solution is cooled to a temperature of 0° C.±3° C.

In some embodiments, the solution is prepared by heating a slurry of the compound of Formula (I) in the solvent component prior to said cooling.

The present application further provides a process for preparing a solid form, which is Form II, comprising evaporating at 25° C. a solution of the compound of Formula (I) in a solvent selected from CH₂Cl₂, CH₃CN, EtOH, and IPA. In some embodiments, the solvent is CH₂Cl₂. In some embodiments, the solvent is CH₃CN. In some embodiments, the solvent is EtOH. In some embodiments, the solvent is IPA.

The present application further provides a process for preparing a solid form, which is Form III, comprising evaporating at 25° C. a solution of the compound of Formula (I) in 1,4-dioxane.

Also provided is a process for preparing Form III comprising preparing a saturated or nearly saturated solution of the compound of Formula (I) in 1,4-dioxane at 25° C.; quench-cooling the solution to a temperature of about −20° C. to about −30° C.; and precipitating the solid form, which is Form III.

The present application further provides a process for preparing a salt form of the compound of Formula (I), which is selected from a mono-maleate salt, a di-besylate salt, a mono-mesylate salt, a di-tosylate salt, a mono-hydrochloride salt, and a di-hydrochloride salt.

Provided in the present application is a process for preparing a mono-maleate salt of a compound of Formula (I), comprising reacting the compound of Formula (I) with maleic acid. In some embodiments, about 1 equivalent to about 2 equivalents of maleic acid are utilized relative to 1 equivalent of the compound of Formula (I). In some embodiments, about 1 equivalent to about 1.5 equivalents of maleic acid are utilized relative to 1 equivalent of the compound of Formula (I). In some embodiments, about 1 equivalent to about 1.2 equivalents of maleic acid are utilized relative to 1 equivalent of the compound of Formula (I). In some embodiments, the reacting of the compound of Formula (I) with the maleic acid is conducted in a solvent component. In some embodiments, the solvent component comprises an alcohol and a halogenated alkane. In some embodiments, the solvent component comprises about 30% to about 70% by weight of a halogenated alkane and about 30% to about 70% by weight of an alcohol. In some embodiments, the solvent component comprises about 40% to about 60% by weight of a halogenated alkane and about 40% to about 60% by weight of an alcohol. In some embodiments, the solvent component comprises about 45% to about 55% by weight of a halogenated alkane and about 45% to about 55% by weight of an alcohol. In some embodiments, the halogenated alkane is a chlorinated alkane. In some embodiments, the solvent component comprises dichloromethane and methanol. In some embodiments, the solvent component comprises about 30% to about 70% by weight of dichloromethane and about 30% to about 70% by weight of methanol. In some embodiments, the solvent component comprises about 40% to about 50% by weight of dichloromethane and about 40% to about 50% by weight of methanol. In some embodiments, the solvent component comprises about 45% to about 55% by weight of dichloromethane and about 45% to about 55% by weight of methanol. In some embodiments, the solvent component comprises 1:1 dichloromethane:methanol.

In some embodiments, after said reacting of the compound of Formula (I) with the maleic acid, the process further comprises removing the solvent component and then slurrying the product of said reacting in acetone.

In some embodiments, the process for preparing a mono-maleate salt of the compound of Formula (I), comprises reacting the compound of Formula (I) with maleic acid in a solvent component comprising dichloromethane and methanol, and then evaporating the solvent component. In some embodiments, the solvent component comprises 1:1 dichloromethane:methanol. In some embodiments, the compound of Formula (I) is dissolved in the solvent component prior to the addition of the maleic acid. In some embodiments, the solvent component is evaporated from the solution at room temperature. In some embodiments, the solution is evaporated to dryness. In some embodiments, evaporating the solvent component results in a solid. In some embodiments, acetone is added to the resulting solid, followed by filtration.

Provided in the present application is a process for preparing a di-besylate salt of a compound of Formula (I), comprising reacting the compound of Formula (I) with benzenesulfonic acid. In some embodiments, about 1 equivalent to about 2 equivalents of benzenesulfonic acid are utilized relative to 1 equivalent of the compound of Formula (I). In some embodiments, about 1 equivalent to about 1.5 equivalents of benzenesulfonic acid are utilized relative to 1 equivalent of the compound of Formula (I). In some embodiments, about 1 equivalent to about 1.2 equivalents of benzenesulfonic acid are utilized relative to 1 equivalent of the compound of Formula (I). In some embodiments, the reacting of the compound of Formula (I) with the benzenesulfonic acid is conducted in a solvent component. In some embodiments, the solvent component comprises an alcohol and a halogenated alkane. In some embodiments, the solvent component comprises about 30% to about 70% by weight of a halogenated alkane and about 30% to about 70% by weight of an alcohol. In some embodiments, the solvent component comprises about 40% to about 60% by weight of a halogenated alkane and about 40% to about 60% by weight of an alcohol. In some embodiments, the solvent component comprises about 45% to about 55% by weight of a halogenated alkane and about 45% to about 55% by weight of an alcohol. In some embodiments, the halogenated alkane is a chlorinated alkane. In some embodiments, the solvent component comprises dichloromethane and methanol. In some embodiments, the solvent component comprises about 30% to about 70% by weight of dichloromethane and about 30% to about 70% by weight of methanol. In some embodiments, the solvent component comprises about 40% to about 50% by weight of dichloromethane and about 40% to about 50% by weight of methanol. In some embodiments, the solvent component comprises about 45% to about 55% by weight of dichloromethane and about 45% to about 55% by weight of methanol. In some embodiments, the solvent component comprises 1:1 dichloromethane:methanol.

In some embodiments, after said reacting of the compound of Formula (I) with the benzenesulfonic acid, the process further comprises removing the solvent component and then slurrying the product of said reacting in acetonitrile.

In some embodiments, after said reacting of the compound of Formula (I) with the benzenesulfonic acid, the process further comprises removing the solvent component and then slurrying the product of said reacting in acetone.

In some embodiments, the process for preparing a di-besylate salt of the compound of Formula (I), comprises reacting the compound of Formula (I) with benzenesulfonic acid in a solvent component comprising dichloromethane and methanol, and then evaporating the solvent component. In some embodiments, the solvent component comprises 1:1 dichloromethane:methanol. In some embodiments, the compound of Formula (I) is dissolved in the solvent component prior to the addition of the benzenesulfonic acid. In some embodiments, the solvent component is evaporated from the solution at room temperature. In some embodiments, the solution is evaporated to a first oil. In some embodiments, acetonitrile is added to the first oil and the solution is evaporated to a second oil. In some embodiments, the acetonitrile is evaporated from the solution at room temperature. In some embodiments, acetone is added to the second oil to form a solution and the solution is slurried to solid. In some embodiments, the solution is slurried at room temperature. In some embodiments, the solid is filtered.

Provided in the present application is a process for preparing a mono-mesylate salt of a compound of Formula (I), comprising reacting the compound of Formula (I) with methanesulfonic acid. In some embodiments, about 1 equivalent to about 2 equivalents of methanesulfonic acid are utilized relative to 1 equivalent of the compound of Formula (I). In some embodiments, about 1 equivalent to about 1.5 equivalents of methanesulfonic acid are utilized relative to 1 equivalent of the compound of Formula (I). In some embodiments, about 1 equivalent to about 1.2 equivalents of methanesulfonic acid are utilized relative to 1 equivalent of the compound of Formula (I). In some embodiments, the reacting of the compound of Formula (I) with methanesulfonic acid is conducted in a solvent component. In some embodiments, the solvent component comprises an alcohol and a halogenated alkane. In some embodiments, the solvent component comprises about 30% to about 70% by weight of a halogenated alkane and about 30% to about 70% by weight of an alcohol. In some embodiments, the solvent component comprises about 40% to about 60% by weight of a halogenated alkane and about 40% to about 60% by weight of an alcohol. In some embodiments, the solvent component comprises about 45% to about 55% by weight of a halogenated alkane and about 45% to about 55% by weight of an alcohol. In some embodiments, the halogenated alkane is a chlorinated alkane. In some embodiments, the solvent component comprises dichloromethane and methanol. In some embodiments, the solvent component comprises about 30% to about 70% by weight of dichloromethane and about 30% to about 70% by weight of methanol. In some embodiments, the solvent component comprises about 40% to about 50% by weight of dichloromethane and about 40% to about 50% by weight of methanol. In some embodiments, the solvent component comprises about 45% to about 55% by weight of dichloromethane and about 45% to about 55% by weight of methanol. In some embodiments, the solvent component comprises 1:1 dichloromethane:methanol.

In some embodiments, after said reacting of the compound of Formula (I) with the methanesulfonic acid, the process further comprises removing the solvent component and then slurrying the product of said reacting in acetone.

In some embodiments, the process for preparing a mono-mesylate salt of the compound of Formula (I), comprises reacting the compound of Formula (I) with methanesulfonic acid in a solvent component comprising dichloromethane and methanol, and then evaporating the solvent component. In some embodiments, the solvent component comprises 1:1 dichloromethane:methanol. In some embodiments, the compound of Formula (I) is dissolved in the solvent component prior to the addition of the methanesulfonic acid. In some embodiments, the solvent component is evaporated from the solution at room temperature. In some embodiments, the solution is evaporated to an oil. In some embodiments, acetone is added to the oil to form a solution and the solution is slurried to solid. In some embodiments, the solution is slurried at room temperature. In some embodiments, the solid is filtered.

Provided in the present application is a process for preparing a di-tosylate salt of a compound of Formula (I), comprising reacting the compound of Formula (I) with p-toluenesulfonic acid. In some embodiments, the p-toluenesulfonic acid is the monohydrate. In some embodiments, about 1 equivalent to about 2 equivalents of p-toluenesulfonic acid are utilized relative to 1 equivalent of the compound of Formula (I). In some embodiments, about 1 equivalent to about 1.5 equivalents of p-toluenesulfonic acid are utilized relative to 1 equivalent of the compound of Formula (I). In some embodiments, about 1 equivalent to about 1.2 equivalents of p-toluenesulfonic acid are utilized relative to 1 equivalent of the compound of Formula (I). In some embodiments, the reacting of the compound of Formula (I) with p-toluenesulfonic acid is conducted in a solvent component. In some embodiments, the solvent component comprises an alcohol and a halogenated alkane. In some embodiments, the solvent component comprises about 30% to about 70% by weight of a halogenated alkane and about 30% to about 70% by weight of an alcohol. In some embodiments, the solvent component comprises about 40% to about 60% by weight of a halogenated alkane and about 40% to about 60% by weight of an alcohol. In some embodiments, the solvent component comprises about 45% to about 55% by weight of a halogenated alkane and about 45% to about 55% by weight of an alcohol. In some embodiments, the halogenated alkane is a chlorinated alkane. In some embodiments, the solvent component comprises dichloromethane and methanol. In some embodiments, the solvent component comprises about 30% to about 70% by weight of dichloromethane and about 30% to about 70% by weight of methanol. In some embodiments, the solvent component comprises about 40% to about 50% by weight of dichloromethane and about 40% to about 50% by weight of methanol. In some embodiments, the solvent component comprises about 45% to about 55% by weight of dichloromethane and about 45% to about 55% by weight of methanol. In some embodiments, the solvent component comprises 1:1 dichloromethane:methanol.

In some embodiments, after said reacting of the compound of Formula (I) with the p-toluenesulfonic acid, the process further comprises removing the solvent component and then slurrying the product of said reacting in acetonitrile.

In some embodiments, after said reacting of the compound of Formula (I) with the p-toluenesulfonic acid, the process further comprises removing the solvent component and then slurrying the product of said reacting in acetone.

In some embodiments, the process for preparing a di-tosylate salt of the compound of Formula (I), comprises reacting the compound of Formula (I) with p-toluenesulfonic acid monohydrate in a solvent component comprising dichloromethane and methanol, and then evaporating the solvent component. In some embodiments, the solvent component comprises 1:1 dichloromethane:methanol. In some embodiments, the compound of Formula (I) is dissolved in the solvent component prior to the addition of the p-toluenesulfonic acid monohydrate. In some embodiments, the solvent component is evaporated from the solution at room temperature. In some embodiments, the solution is evaporated to an oil. In some embodiments, acetonitrile is added to the first oil and the solution is evaporated to an oil/semi-solid. In some embodiments, the acetonitrile is evaporated from the solution at room temperature. In some embodiments, acetone is added to the oil/semi-solid to form a solution and the solution is slurried to solid. In some embodiments, the solution is slurried at room temperature. In some embodiments, the solid is filtered.

Provided in the present application is a process for preparing a mono-hydrochloride salt of a compound of Formula (I), comprising reacting the compound of Formula (I) with hydrochloric acid. In some embodiments, about 1 equivalent to about 2 equivalents of hydrochloric acid are utilized relative to 1 equivalent of the compound of Formula (I). In some embodiments, about 1 equivalent to about 1.5 equivalents of hydrochloric acid are utilized relative to 1 equivalent of the compound of Formula (I). In some embodiments, about 1 equivalent to about 1.2 equivalents of hydrochloric acid are utilized relative to 1 equivalent of the compound of Formula (I). In some embodiments, the reacting of the compound of Formula (I) with hydrochloric acid is conducted in a solvent component. In some embodiments, the solvent component comprises an alcohol and a halogenated alkane. In some embodiments, the solvent component comprises about 30% to about 70% by weight of a halogenated alkane and about 30% to about 70% by weight of an alcohol. In some embodiments, the solvent component comprises about 40% to about 60% by weight of a halogenated alkane and about 40% to about 60% by weight of an alcohol. In some embodiments, the solvent component comprises about 45% to about 55% by weight of a halogenated alkane and about 45% to about 55% by weight of an alcohol. In some embodiments, the halogenated alkane is a chlorinated alkane. In some embodiments, the solvent component comprises dichloromethane and methanol. In some embodiments, the solvent component comprises about 30% to about 70% by weight of dichloromethane and about 30% to about 70% by weight of methanol. In some embodiments, the solvent component comprises about 40% to about 50% by weight of dichloromethane and about 40% to about 50% by weight of methanol. In some embodiments, the solvent component comprises about 45% to about 55% by weight of dichloromethane and about 45% to about 55% by weight of methanol. In some embodiments, the solvent component comprises 1:1 dichloromethane:methanol.

In some embodiments, after said reacting of the compound of Formula (I) with the hydrochloric acid, the process further comprises removing the solvent component and then slurrying the product of said reacting in acetone.

In some embodiments, the process for preparing a mono-hydrochloride salt of the compound of Formula (I), comprises reacting the compound of Formula (I) with about 1 to about 1.5 equivalents of hydrochloric acid in a solvent component comprising dichloromethane and methanol, and then evaporating the solvent component. In some embodiments, the solvent component comprises 1:1 dichloromethane:methanol. In some embodiments, the compound of Formula (I) is dissolved in the solvent component prior to the addition of the hydrochloric acid. In some embodiments, the hydrochloric acid is 6 M aqueous hydrochloric acid. In some embodiments, the solvent component is evaporated from the solution at room temperature. In some embodiments, the solution is evaporated to dryness. In some embodiments, evaporating the solvent component results in a solid.

Provided in the present application is a process for preparing a di-hydrochloride salt of a compound of Formula (I), comprising reacting the compound of Formula (I) with hydrochloric acid. In some embodiments, about 2 equivalent to about 3 equivalents of hydrochloric acid are utilized relative to 1 equivalent of the compound of Formula (I). In some embodiments, about 2 equivalent to about 2.5 equivalents of hydrochloric acid are utilized relative to 1 equivalent of the compound of Formula (I). In some embodiments, about 2 equivalent to about 2.2 equivalents of hydrochloric acid are utilized relative to 1 equivalent of the compound of Formula (I). In some embodiments, the reacting of the compound of Formula (I) with hydrochloric acid is conducted in a solvent component. In some embodiments, the solvent component comprises an alcohol and a halogenated alkane. In some embodiments, the solvent component comprises about 30% to about 70% by weight of a halogenated alkane and about 30% to about 70% by weight of an alcohol. In some embodiments, the solvent component comprises about 40% to about 60% by weight of a halogenated alkane and about 40% to about 60% by weight of an alcohol. In some embodiments, the solvent component comprises about 45% to about 55% by weight of a halogenated alkane and about 45% to about 55% by weight of an alcohol. In some embodiments, the halogenated alkane is a chlorinated alkane. In some embodiments, the solvent component comprises dichloromethane and methanol. In some embodiments, the solvent component comprises about 30% to about 70% by weight of dichloromethane and about 30% to about 70% by weight of methanol. In some embodiments, the solvent component comprises about 40% to about 50% by weight of dichloromethane and about 40% to about 50% by weight of methanol. In some embodiments, the solvent component comprises about 45% to about 55% by weight of dichloromethane and about 45% to about 55% by weight of methanol. In some embodiments, the solvent component comprises 1:1 dichloromethane:methanol.

In some embodiments, after said reacting of the compound of Formula (I) with the hydrochloric acid, the process further comprises removing the solvent component and then slurrying the product of said reacting in acetone.

In some embodiments, the process for preparing a di-hydrochloride salt of the compound of Formula (I), comprises reacting the compound of Formula (I) with about equivalents to about 2.5 equivalents of hydrochloric acid in a solvent component comprising dichloromethane and methanol, and then evaporating the solvent component. In some embodiments, the solvent component comprises 1:1 dichloromethane:methanol. In some embodiments, the compound of Formula (I) is dissolved in the solvent component prior to the addition of the hydrochloric acid. In some embodiments, the hydrochloric acid is 6 M aqueous hydrochloric acid. In some embodiments, the solvent component is evaporated from the solution at room temperature. In some embodiments, the solution is evaporated to dryness.

In some embodiments, acetone is added to the resulting solid, followed by filtration.

The present application also provides processes for preparing a compound of Formula (I), or a solid form or a salt thereof. Accordingly, the present application provides a process of preparing a compound of Formula (I), or a pharmaceutically acceptable salt thereof; a solid form of the compound of Formula (I), which is Form I, Form II, or Form III; or any of the salts of the compound of Formula (I) as described herein, comprising:
reacting a compound of Formula (1c):

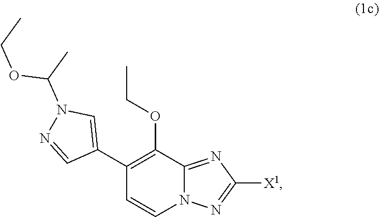

with a compound of Formula (1b):

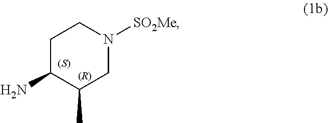

or a salt thereof, via a Buchwald coupling reaction to form a compound of Formula (1a):

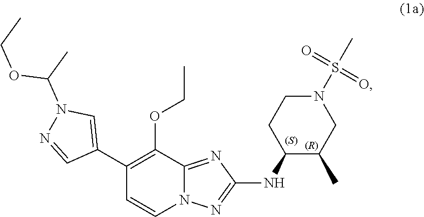

wherein X¹ is halo.

In some embodiments, X¹ is Br.

In some embodiments, the compound of Formula (1b), or the salt thereof, is the HCl salt. In some embodiments, the Buchwald coupling reaction comprises reacting the compound of Formula (1c) with the compound of Formula (1b), or the salt thereof, in the presence of a Buchwald catalyst or precatalyst and a base.

In some embodiments, the Buchwald catalyst or precatalyst is a palladium catalyst. In some embodiments, the palladium catalyst or precatalyst is [(2-di-tert-butylphosphino-3,6-dimethoxy-2′,4′,6′-triisopropyl-1,1′-biphenyl)-2-(2′-amino-1,1′-biphenyl)]palladium(II) methanesulfonate (t-BuBrett Phos Pd G3), [tBuBrettPhos Pd(allyl)]OTf (Pd-175), chloro(2-dicyclohexylphosphino-2′,4′,6′-triisopropyl-1,1′-biphenyl)[2-(2′-amino-1,1′-biphenyl)]palladium(II) (XPhos-Pd-G2), [(4,5-bis(diphenylphosphino)-9,9-dimethylxanthene)-2-(2′-amino-1,1′-biphenyl)]palladium(II) methanesulfonate (XantPhos Pd G3), tBuBrettPhos Pd G3, SPhos Pd G3, cataCXium® A Pd G3, BrettPhos Pd G3, tBuXPhos Pd G3, [(1,3,5,7-tetramethyl-6-phenyl-2,4,6-trioxa-6-phosphaadamantane)-2-(2′-amino-1,1′-biphenyl)]palladium(II) methanesulfonate, JackiePhos Pd G3, CPhos Pd G3, RuPhos Pd G3, APhos Pd G3, RockPhos Pd G3, AdBrettPhos Pd G3, Neopentyl(t-Bu)2P Pd G3, TrixiePhos Pd G3, N-XantPhos Pd G3, DTBPF-Pd-G3, DPPF Pd G3, DavePhos-Pd-G3, (t-Bu)₂PhP Pd G3, rac-BINAP-Pd-G3, CyJohnPhos Pd G3, MorDalphos Pd G3, Josiphos SL-J009-1 Pd G3, P(Cy₃) Pd G3, Me3(OMe)tBuXPhos-Pd-G3, 4MetBuXPhos Pd G3, (t-Bu)PhCPhos Pd G3, CyJohnPhos Pd G3, mesyl[(tri-t-butylphosphine)-2-(2-aminobiphenyl)]palladium(II), DTBPF-Pd-G3, P(o-tol)₃ Pd G3, VPhos Pd G3, QPhos Pd G3, RuPhos Pd G4, SPhos Pd G4, BrettPhos Pd G4, XPhos Pd G4, APhos Pd G4, rac-BINAP Pd G4, P(t-Bu)₃ Pd G4, (t-Bu)PhCPhos Pd G4, cataCXium Pd G4, CPhos Pd G4, CyJohnPhos Pd G4, DavePhos Pd G4, DPPF Pd G4, EPhos Pd G4, MorDalPhos Pd G4, neopentyl(tBu)2P Pd G4, PCy₃ Pd G4, (tBu)₂PMe Pd G4, (tBu)₂PPh Pd G4, 1,3,5,7-tetramethyl-6-phenyl-2,4,6-trioxa-6-phosphaadamantane Pd G4, (R)-TolBINAP Pd G4, VPhos Pd G4, XantPhos Pd G4, N-XantPhos Pd G4, t-BuDavePhos Pd G4, XPhos Pd G2, RuPhos Pd G2, SPhos Pd G2, bis(triphenylphosphine)palladium(II) dichloride, tBuXPhos Pd G1, RuPhos Pd G1 methyl t-butyl ether adduct, SPhos Pd G1 methyl t-butyl ether adduct, 2′-(dimethylamino)-2-biphenylyl-palladium(II) chloride dinorbornylphosphine complex, chloro(η2-P,C-tris(2,4-di-tert-butylphenyl)phosphite)(tricyclohexylphosphine)palladium(II), di-μ-chlorobis[5-chloro-2-[(4-chlorophenyl)(hydroxyimino-κN)methyl]phenyl-κC]palladium dimer, DavePhos Pd G2, (Ad-BippyPhos)₂PdCl₂, APhos Pd G2, sSPhos Pd G2, P(t-Bu)₃ Pd G3, BrettPhos Pd G1 methyl t-butyl ether adduct, dichloro[2-(4,5-dihydro-2-oxazolyl)quinoline]palladium(II), salicylaldehyde thiosemicarbazone palladium(II) chloride, XPhos Pd G1, bis[(dicyclohexyl)(4-dimethylaminophenyl)phosphine]palladium(II) chloride, bis(di-tert-butyl(4-dimethylaminophenyl)phosphine)dichloropalladium(II), di-μ-chlorobis[5-hydroxy-2-[1-(hydroxyimino-κN)ethyl]phenyl-κC]palladium(II) dimer, 2-(2′-di-tert-butylphosphine)biphenylpalladium(II) acetate, or 2-(dimethylaminomethyl)ferrocen-1-yl-palladium(II) chloride dinorbornylphosphine complex.

In some embodiments, the palladium catalyst or precatalyst is XPhos Pd G3.

In some embodiments, the base is an alkali metal alkoxide. In some embodiments, the base is sodium t-butoxide.

In some embodiments, about 1 to about 1.5 equivalents of the compound of Formula (1b), or the salt thereof, is utilized relative to 1 equivalent of the compound of Formula (1c). In some embodiments, about 1.2 equivalents of the compound of Formula (1b), or the salt thereof, is utilized relative to 1 equivalent of the compound of Formula (1c).

In some embodiments, about 4 to about 6 equivalents of the base is utilized relative to 1 equivalent of the compound of Formula (1c).

In some embodiments, about 0.0001 to about 0.1 equivalents of the Buchwald catalyst or precatalyst is utilized relative to 1 equivalent of the compound of Formula (1c). In some embodiments, about 0.01 to about 0.05 equivalents of the Buchwald catalyst or precatalyst is utilized relative to 1 equivalent of the compound of Formula (1c).

In some embodiments, the reacting of the compound of Formula (1c) with the compound of Formula (1b), or the salt thereof, is conducted at a temperature of from about 80° C. to about 100° C. In some embodiments, the reacting of the compound of Formula (1c) with the compound of Formula (1b), or the salt thereof, is conducted at a temperature of about 90° C.

In some embodiments, the reacting of the compound of Formula (1c) with the compound of Formula (1b), or the salt thereof, is conducted in a solvent component. In some embodiments, the solvent component for the reacting of the compound of Formula (1c) with the compound of Formula (1b), or the salt thereof, comprises a cyclic ether. In some embodiments, the solvent component for the reacting of the compound of Formula (1c) with the compound of Formula (1b), or the salt thereof, comprises dioxane.

In some embodiments, the process further comprises reacting the compound of Formula (1a) with an organic acid to form a salt of the compound of Formula (1a).

In some embodiments, the organic acid is succinic acid.

In some embodiments, the salt of the compound of Formula (1) is a hemi-succinic acid salt of the compound of Formula (1a).

In some embodiments, the process further comprises reacting the compound of Formula (1a) with succinic acid to form a hemi-succinic acid salt of the compound of Formula (1a).

In some embodiments, from about 2 equivalents to about 2.5 equivalents of succinic acid are utilized relative to 1 equivalent of the compound of Formula (1a).

In some embodiments, the reacting of the compound of Formula (1a) with succinic acid is conducted in a solvent component. In some embodiments the solvent component for the reacting of the compound of Formula (1a) with succinic acid comprises acetonitrile.

In some embodiments, the reacting of the compound of Formula (1a) with succinic acid is conducted at a temperature of from about 50° C. to about 60° C.

In some embodiments, the process further comprises deprotecting the compound of Formula (1a), or a salt thereof, to form the compound of Formula (I).

In some embodiments, the process further comprises deprotecting the compound of Formula (1a) to form the compound of Formula (I).

In some embodiments, the deprotecting is accomplished by reacting the compound of Formula (1a) with a strong acid. In some embodiments, the strong acid is hydrochloric acid.

In some embodiments, about 2 to about 3 equivalents of the strong acid is utilized relative to 1 equivalent of the compound of Formula (1a).

In some embodiments, the deprotecting of the compound of Formula (1a) is conducted at a temperature of from about 50° C. to about 70° C. In some embodiments, the deprotecting of the compound of Formula (1a) is conducted at a temperature of about 60° C.

In some embodiments, the deprotecting of the compound of Formula (1a) is conducted in a solvent component. In some embodiments, the solvent component for deprotecting of the compound of Formula (1a) comprises a cyclic ether. In some embodiments, the solvent component for deprotecting of the compound of Formula (1a) comprises tetrahydrofuran (THF).

In some embodiments, the compound of Formula (1c) is prepared by a process comprising:
reacting a compound of Formula (1d):

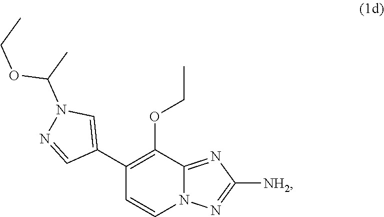 [SMILES: CCOc1c(-c2cnn(C(C)OCC)c2)ccn2nc(N)nc12]

or a salt thereof, with a halogenating agent to form the compound of Formula (1c).

In some embodiments, the halogenating agent is a brominating agent.

In some embodiments, the halogenating agent is Cu(X¹)₂.

In some embodiments, the halogenating agent is CuBr₂.

In some embodiments, about 1 to about 1.5 equivalents of a halogenating agent is utilized relative to 1 equivalent of the compound of Formula (1d), or the salt thereof. In some embodiments, about 1 to about 1.1 equivalents of a halogenating agent is utilized relative to 1 equivalent of the compound of Formula (1d), or the salt thereof.

In some embodiments, the reacting of the compound of Formula (1d), or the salt thereof, with the halogenating agent, is conducted at a temperature of from about 0° C. to about 10° C. In some embodiments, the reacting of the compound of Formula (1d), or the salt thereof, with the halogenating agent, is conducted at a temperature of about 5° C., followed by warming to room temperature.

In some embodiments, the reacting of the compound of Formula (i d), or the salt thereof, with the halogenating agent, is conducted in a solvent component. In some embodiments, the solvent component for the reacting of the compound of Formula (1d), or the salt thereof, with the halogenating agent comprises acetonitrile.

In some embodiments, the compound of Formula (1d), or the salt thereof, is prepared by a process comprising:
reacting a compound of Formula 1(e):

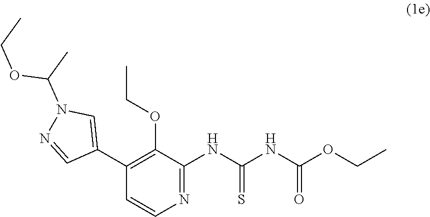 [SMILES: CCOC(=O)NC(=S)Nc1nccc(-c2cnn(C(C)OCC)c2)c1OCC]

with hydroxylamine HCl and a base component to form the compound of formula (1d), or the salt thereof.

In some embodiments, the base component is a tertiary amine. In some embodiments, the tertiary amine is ethyldiisopropylamine.

In some embodiments, about 1 equivalent to about 2 equivalents of hydroxylamine HCl are utilized relative to about 1 equivalent of the compound of Formula (1e).

In some embodiments, the reacting of the compound of Formula (1e), or the salt thereof, with hydroxylamine HCl and a base component, is conducted at a temperature of from about 40° C. to about 60° C. In some embodiments, the reacting of the compound of Formula (1e), or the salt thereof, with hydroxylamine HCl and a base component, is conducted at a temperature of about 50° C.

In some embodiments, the reacting of the compound of Formula (1e), or the salt thereof, with hydroxylamine HCl and a base component, is conducted in a solvent component. In some embodiments, the solvent component for the reacting of the compound of Formula (1e), or the salt thereof, with hydroxylamine HCl and a base component comprises an alcohol. In some embodiments, the solvent component for the reacting of the compound of Formula (1e), or the salt thereof, with hydroxylamine HCl and a base component comprises an alcohol. In some embodiments, the solvent component for the reacting of the compound of Formula (1e), or the salt thereof, with hydroxylamine HCl and a base component comprises ethanol.

In some embodiments, the compound of Formula (1e) is prepared by a process comprising:
reacting a compound of Formula (1f):

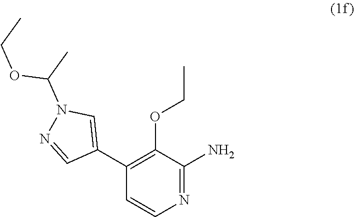 [SMILES: CCOc1c(-c2cnn(C(C)OCC)c2)ccnc1N]

with CH₃CH₂OC(O)—N═C═S to form the compound of Formula (1e).

In some embodiments, about 1 to about 1.5 equivalents of the CH₃CH₂OC(O)—N═C═S are used relative to the compound of Formula (1f).

In some embodiments, the reacting of the compound of Formula (1f) with CH₃CH₂OC(O)—N═C═S is conducted at a temperature of from about 0° C. to about 20° C., followed by warming to room temperature. In some embodiments, the reacting of the compound of Formula (1f) with CH₃CH₂OC(O)—N═C═S is conducted at a temperature of about 10° C., followed by warming to room temperature.

In some embodiments, the reacting of the compound of Formula (1f) with CH₃CH₂OC(O)—N═C═S is conducted in a solvent component. In some embodiments, the solvent component for the reacting of the compound of Formula (1f) with CH₃CH₂OC(O)—N═C═S comprises a cyclic ether. In some embodiments, the solvent component for the reacting of the compound of Formula (1f) with CH₃CH₂OC(O)—N═C═S comprises dioxane.

In some embodiments, the compound of Formula (1f) is prepared by a process comprising:
reacting a compound of Formula (1h):

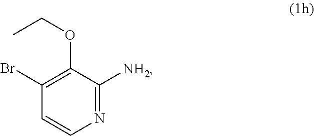

or a salt thereof, with a compound of Formula (1g):

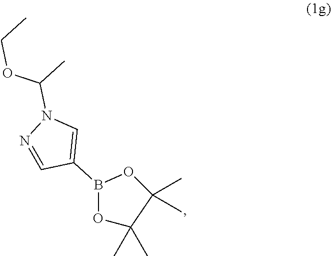 [SMILES: CCOC(C)n1cc(B2OC(C)(C)C(C)(C)O2)cn1]

via a Buchwald coupling reaction to form the compound of Formula (1f).

In some embodiments, the Buchwald coupling reaction comprises reacting the compound of Formula (1h), or the salt thereof, with the compound of Formula (1g) in the presence of Buchwald catalyst or precatalyst and a base.

In some embodiments, the Buchwald catalyst or precatalyst is a palladium catalyst. In some embodiments, the palladium catalyst or precatalyst is [(2-di-tert-butylphosphino-3, 6-dimethoxy-2′,4′,6′-triisopropyl-1,1′-biphenyl)-2-(2′-amino-1,1′-biphenyl)]palladium(II) methanesulfonate (t-BuBrett Phos Pd G3), [tBuBrettPhos Pd(allyl)]OTf (Pd-175), chloro(2-dicyclohexylphosphino-2′,4′,6′-triisopropyl-1,1′-biphenyl)[2-(2′-amino-1,1′-biphenyl)]palladium(II) (XPhos-Pd-G2), [(4,5-bis(diphenylphosphino)-9,9-dimethylxanthene)-2-(2′-amino-1,1′-biphenyl)]palladium(II) methanesulfonate (XantPhos Pd G3), tBuBrettPhos Pd G3, SPhos Pd G3, cataCXium® A Pd G3, BrettPhos Pd G3, tBuXPhos Pd G3, [(1,3,5,7-tetramethyl-6-phenyl-2,4,6-trioxa-6-phosphaadamantane)-2-(2′-amino-1,1′-biphenyl)]palladium(II) methanesulfonate, JackiePhos Pd G3, CPhos Pd G3, RuPhos Pd G3, APhos Pd G3, RockPhos Pd G3, AdBrettPhos Pd G3, Neopentyl(t-Bu)2P Pd G3, TrixiePhos Pd G3, N-XantPhos Pd G3, DTBPF-Pd-G3, DPPF Pd G3, DavePhos-Pd-G3, (t-Bu)₂PhP Pd G3, rac-BINAP-Pd-G3, CyJohnPhos Pd G3, MorDalphos Pd G3, Josiphos SL-J009-1 Pd G3, P(Cy₃) Pd G3, Me3(OMe)tBuXPhos-Pd-G3, 4MetBuXPhos Pd G3, (t-Bu)PhCPhos Pd G3, CyJohnPhos Pd G3, mesyl[(tri-t-butylphosphine)-2-(2-aminobiphenyl)]palladium(II), DTBPF-Pd-G3, P(o-tol)₃ Pd G3, VPhos Pd G3, QPhos Pd G3, RuPhos Pd G4, SPhos Pd G4, BrettPhos Pd G4, XPhos Pd G4, APhos Pd G4, rac-BINAP Pd G4, P(t-Bu)₃ Pd G4, (t-Bu)PhCPhos Pd G4, cataCXium Pd G4, CPhos Pd G4, CyJohnPhos Pd G4, DavePhos Pd G4, DPPF Pd G4, EPhos Pd G4, MorDalPhos Pd G4, neopentyl(tBu)2P Pd G4, PCy₃ Pd G4, (tBu)₂PMe Pd G4, (tBu)₂PPh Pd G4, 1,3,5,7-tetramethyl-6-phenyl-2,4,6-trioxa-6-phosphaadamantane Pd G4, (R)-TolBINAP Pd G4, VPhos Pd G4, XantPhos Pd G4, N-XantPhos Pd G4, t-BuDavePhos Pd G4, XPhos Pd G2, RuPhos Pd G2, SPhos Pd G2, bis(triphenylphosphine)palladium(II) dichloride, tBuXPhos Pd G1, RuPhos Pd G1 methyl t-butyl ether adduct, SPhos Pd G1 methyl t-butyl ether adduct, 2′-(dimethylamino)-2-biphenylyl-palladium(II) chloride dinorbornylphosphine complex, chloro(η2-P,C-tris(2,4-di-tert-butylphenyl)phosphite)(tricyclohexylphosphine)palladium(II), di-μ-chlorobis[5-chloro-2-[(4-chlorophenyl)(hydroxyimino-κN)methyl]phenyl-κC]palladium dimer, DavePhos Pd G2, (Ad-BippyPhos)₂PdCl₂, APhos Pd G2, sSPhos Pd G2, P(t-Bu)₃ Pd G3, BrettPhos Pd G1 methyl t-butyl ether adduct, dichloro[2-(4,5-dihydro-2-oxazolyl)quinoline]palladium(II), salicylaldehyde thiosemicarbazone palladium(II) chloride, XPhos Pd G1, bis[(dicyclohexyl)(4-dimethylaminophenyl)phosphine]palladium(II) chloride, bis(di-tert-butyl(4-dimethylaminophenyl)phosphine)dichloropalladium(II), di-μ-chlorobis[5-hydroxy-2-[1-(hydroxyimino-κN)ethyl]phenyl-κC]palladium(II) dimer, 2-(2′-di-tert-butylphosphine)biphenylpalladium(II) acetate, or 2-(dimethylaminomethyl)ferrocen-1-yl-palladium(II) chloride dinorbornylphosphine complex.

In some embodiments, the compound of Formula (1h), or the salt thereof, is the HBr salt.

In some embodiments, the Buchwald catalyst or precatalyst, present for the reacting of the compound of Formula (1h), or the salt thereof, and the compound of Formula (1g), is a palladium catalyst. In some embodiments, the Buchwald catalyst or precatalyst, present for the reacting of the compound of Formula (1h), or the salt thereof, and the compound of Formula (1g), is (2-dicyclohexylphosphino-2′,4′,6′-triisopropyl-1,1′-biphenyl)[2-(2′-amino-1,1′-biphenyl)]palladium(II) methanesulfonate (XPhos Pd G3).

In some embodiments, the base, present for the reacting of the compound of Formula (1h), or the salt thereof, and the compound of Formula (1g), is an alkali metal phosphate. In some embodiments, the base, present for the reacting of the compound of Formula (1h), or the salt thereof, and the compound of Formula (1g), is sodium phosphate tribasic.

In some embodiments, the reacting of the compound of Formula (1h), or the salt thereof, with the compound of Formula (1g), is conducted at a temperature of from about 75° C. to about 95° C. In some embodiments, the reacting of the compound of Formula (1h), or the salt thereof, with the compound of Formula (1g), is conducted at a temperature of about 85° C.

In some embodiments, the reacting of the compound of Formula (1h), or the salt thereof, with the compound of Formula (1g), is conducted in a solvent component. In some embodiments, the solvent component for the reacting of the compound of Formula (1h), or the salt thereof, with the compound of Formula (1g) comprises water and a cyclic ether. In some embodiments, the solvent component for the reacting of the compound of Formula (1h), or the salt thereof, with the compound of Formula (1g) comprises water and dioxane.

In some embodiments, about 1 to about 1.5 equivalents of the compound of Formula (1g) are utilized relative to 1 equivalent of the compound of Formula (1h).

In some embodiments, about 2 to about 4 equivalents of the base is utilized relative to 1 equivalent of the compound of Formula (1h).

In some embodiments, about 0.0001 to about 0.1 equivalents of the Buchwald catalyst or precatalyst is utilized relative to 1 equivalent of the compound of Formula (1h). In some embodiments, about 0.001 to about 0.005 equivalents of the Buchwald catalyst or precatalyst is utilized relative to 1 equivalent of the compound of Formula (1h).

In some embodiments, the compound of Formula (1h), or the salt thereof, is prepared by a process comprising:
reacting a compound of Formula (1i):

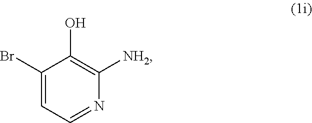

or a salt thereof, with an ethyl halide in the presence of a base to form the compound of Formula (1h), or the salt thereof.

In some embodiments, the ethyl halide is ethyl iodide.

In some embodiments, about 1 equivalent to about 2 equivalents of the ethyl halide are utilized relative to the compound of Formula (1i), or the salt thereof.

In some embodiments, about 2 equivalent to about 3 equivalents of the base are utilized relative to the compound of Formula (1i), or the salt thereof.

In some embodiments, the base present for the reacting of the compound of Formula (1i), or the salt thereof, with the ethyl halide is a carbonate base. In some embodiments, the carbonate base is cesium carbonate. In some embodiments, the compound of Formula (1i), or the salt thereof, is a HBr salt.

In some embodiments, the reacting of the compound of Formula (1i), or the salt thereof, with the ethyl halide, is conducted at a temperature of from about 55° C. to about 80° C. In some embodiments, the reacting of the compound of Formula (1i), or the salt thereof, with the ethyl halide, is conducted at a temperature of about 65° C. to about 70° C.

In some embodiments, the reacting of the compound of Formula (1i), or the salt thereof, with the ethyl halide, is conducted in a solvent component. In some embodiments, the solvent component for the reacting of the compound of Formula (1i), or the salt thereof, with the ethyl halide comprises acetonitrile.

In some embodiments, the compound of Formula (1c) is prepared by a process comprising:
reacting a compound of Formula (2a):

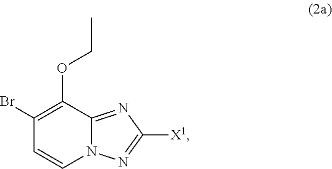

with a compound of Formula (2b):

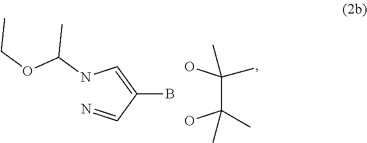

to form the compound of Formula (1c), in the presence of a Suzuki catalyst and a base, wherein X¹ is halo.

In some embodiments, X¹ is Br.

In some embodiments, about 1 to about 1.5 equivalents of the compound of Formula (2b) are utilized relative to 1 equivalent of the compound of Formula (2a).

In some embodiments, the Suzuki catalyst is a palladium catalyst. In some embodiments, the Suzuki catalyst is formed from a mixture of a phosphine ligand and a palladium (II) compound. In some embodiments, the Suzuki catalyst is formed from a mixture of CataCXium A and palladium acetate.

In some embodiments, the base, present for the reacting of the compound of Formula (2a) and the compound of Formula (2b), is an alkali metal phosphate. In some embodiments, the base, present for the reacting of the compound of Formula (2a) and the compound of Formula (2b), is sodium phosphate tribasic.

In some embodiments, the reacting of the compound of Formula (2a) with the compound of Formula (2b), is conducted at a temperature of from about 40° C. to about 60° C. In some embodiments, the reacting of the compound of Formula (2a) with the compound of Formula (2b), is conducted at a temperature of about 50° C.

In some embodiments, the reacting of the compound of Formula (2a) with the compound of Formula (2b), is conducted in a solvent component. In some embodiments, the solvent component for the reacting of the compound of Formula (2a) with the compound of Formula (2b) comprises a cyclic ether. In some embodiments, the solvent component for the reacting of the compound of Formula (2a) with the compound of Formula (2b) comprises dioxane.

In some embodiments, the compound of Formula (1b), or the salt thereof, is prepared by a process comprising:
reducing a compound of Formula (3a):

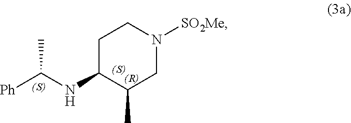

to form the compound of Formula (1b), or the salt thereof.

In some embodiments, the reducing is accomplished by reacting the compound of Formula (3a) with hydrogen gas in the presence of a palladium catalyst. In some embodiments, the reducing is accomplished by reacting the compound of Formula (3a) with hydrogen gas in the presence of a Pd(OH)₂.

In some embodiments, the reducing of the compound of Formula (3a) is conducted at room temperature.

In some embodiments, the reducing of the compound of Formula (3a) is conducted in a solvent component. In some embodiments, the solvent component for the reducing of the compound of Formula (3a) comprises an alcohol. In some embodiments, the solvent component for the reducing of the compound of Formula (3a) comprises methanol.

In some embodiments, the compound of Formula (3a) is prepared by a process comprising:
reacting a compound of Formula (3c):

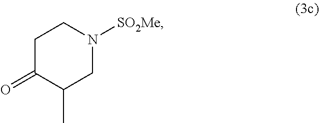

with a compound of Formula (3b):

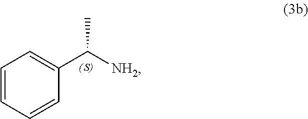

followed by crystallization to give the compound of Formula (3a).

In some embodiments, about 1 to about 2 equivalents of the compound of Formula (3b) are utilized relative to 1 equivalent of the compound of Formula (3c).

In some embodiments, the reacting of the compound of Formula (3c) with the compound of Formula (3b) is conducted in the presence of a coupling agent and a base. In some embodiments, the coupling agent, present for reacting of the compound of Formula (3c) with the compound of Formula (3b), is a borohydride. In some embodiments, the coupling agent, present for reacting of the compound of Formula (3c) with the compound of Formula (3b), is NaBH(OAc)₂. In some embodiments, the base, present for the reacting of the compound of Formula (3c) with the compound of Formula (3b), is a tertiary amine. In some embodiments, the base, present for the reacting of the compound of Formula (3c) with the compound of Formula (3b) is diisopropylethylamine.

In some embodiments, the reacting of the compound of Formula (3c) with the compound of Formula (3b), is conducted at a temperature of from about 10° C. to about 35° C. In some embodiments, the reacting of the compound of Formula (3c) with the compound of Formula (3b), is conducted at a temperature of about 20° C. to about 25° C.

In some embodiments, the reacting of the compound of Formula (3c) with the compound of Formula (3b), is conducted in a solvent component. In some embodiments, the solvent component for the reacting of the compound of Formula (3c) with the compound of Formula (3b) comprises a cyclic ether. In some embodiments, the solvent component for the reacting of the compound of Formula (3c) with the compound of Formula (3b) comprises tetrahydrofuran.

In some embodiments, the crystallization is conducted by dissolving the product of the reacting of the compound of Formula (3c) with the compound of Formula (3b) in a solvent component and then cooling the solution to form the compound of Formula (3c). In some embodiments, the solvent component for the dissolving is ethyl acetate (EtOAc).

In some embodiments, the compound of Formula (3c) is prepared by a process comprising:
reacting a compound of Formula (3d):

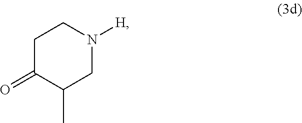

or a salt thereof, with methanesulfonyl chloride to form a compound of Formula (3c).

In some embodiments, the reacting of the compound of Formula (3d) with the methanesulfonyl chloride, is conducted in the presence of a base. In some embodiments, the base, present for the reacting of the compound of Formula (3d) with the methanesulfonyl chloride, is a tertiary amine. In some embodiments, the base, present for the reacting of the compound of Formula (3d) with the methanesulfonyl chloride, is triethylamine.

In some embodiments, about 1 to about 1.5 equivalents of the methanesulfonyl chloride are utilized relative to 1 equivalent of the compound of Formula (3d), or the salt thereof.

In some embodiments, the reacting of the compound of Formula (3d) with the methanesulfonyl chloride, is conducted at room temperature.

In some embodiments, the reacting of the compound of Formula (3d) with the methanesulfonyl chloride, is conducted in a solvent component. In some embodiments, the solvent component for the reacting of the compound of Formula (3d) with the methanesulfonyl chloride comprises dichloromethane.

In some embodiments, the present application also provides a compound selected from:

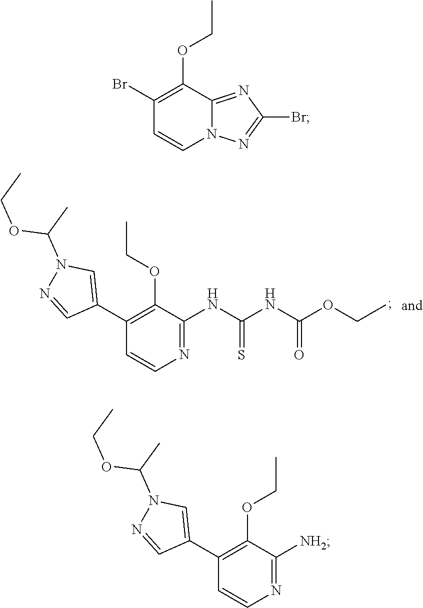 [SMILES: CCOc1c(Br)ccn2nc(Br)nc12 ; CCOC(=O)NC(=S)Nc1nccc(-c2cnn(C(C)OCC)c2)c1OCC ; CCOc1c(-c2cnn(C(C)OCC)c2)ccnc1N]

or a salt thereof.

In some embodiments, the present application provides a hemi-succinate salt of a compound of Formula (1a):

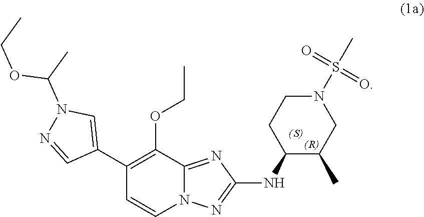

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, “halo” refers to F, Cl, Br, or I. In some embodiments, halo is F, Cl, or Br. In some embodiments, halo is F or Cl. In some embodiments, halo is F. In some embodiments, halo is Cl.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The processes described herein can also be used to prepare pharmaceutically acceptable salts of the compound of Formula (I). As used herein, the term “pharmaceutically acceptable salt” refers to a salt formed by the addition of a pharmaceutically acceptable acid or base to a compound disclosed herein. As used herein, the phrase “pharmaceutically acceptable” refers to a substance that is acceptable for use in pharmaceutical applications from a toxicological perspective and does not adversely interact with the active ingredient. Pharmaceutically acceptable salts, including mono- and bi-salts, include, but are not limited to, those derived from organic and inorganic acids such as, but not limited to, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, and similarly known acceptable acids. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in their entireties.

The reactions for preparing compounds described herein can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

The expressions, “ambient temperature” or “room temperature” or “r.t.” as used herein, are understood in the art, and refer generally to a temperature, e.g., a reaction temperature, that is about the temperature of the room in which the reaction is carried out, for example, a temperature from about 20° C. to about 30° C.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

### Methods of Use

The solid form and salts of the present disclosure can inhibit CDK2 and therefore are useful for treating diseases wherein the underlying pathology is, wholly or partially, mediated by CDK2. Such diseases include cancer and other diseases with proliferation disorder. In some embodiments, the present disclosure provides treatment of an individual or a patient in vivo using the solid form and salts of the present disclosure such that growth of cancerous tumors is inhibited. The solid form and salts described herein can be used to inhibit the growth of cancerous tumors with aberrations that activate the CDK2 kinase activity. These include, but are not limited to, disease (e.g., cancers) that are characterized by amplification or overexpression of CCNE1 such as ovarian cancer, uterine carcinosarcoma and breast cancer and p27 inactivation such as breast cancer and melanomas. Accordingly, in some embodiments of the methods, the patient has been previously determined to have an amplification of the cyclin E1 (CCNE1) gene and/or an expression level of CCNE1 in a biological sample obtained from the human subject that is higher than a control expression level of CCNE1. Alternatively, the solid form and salts described herein can be used in conjunction with other agents or standard cancer treatments, as described below. In one embodiment, the present disclosure provides a method for inhibiting growth of tumor cells in vitro. The method includes contacting the tumor cells in vitro with solid form and salts. In another embodiment, the present disclosure provides a method for inhibiting growth of tumor cells with CCNE1 amplification and overexpression in an individual or a patient. The method includes administering to the individual or patient in need thereof a therapeutically effective amount of solid form and salts described herein.

In some embodiments, provided herein is a method of inhibiting CDK2, comprising contacting the CDK2 with the solid form and salts described herein. In some embodiments, provided herein is a method of inhibiting CDK2 in a patient, comprising administering to the patient the solid form and salts described herein.

In some embodiments, provided herein is a method for treating cancer. The method includes administering to a patient (in need thereof), a therapeutically effective amount of the solid form and salts described herein. In another embodiment, the cancer is characterized by amplification or overexpression of CCNE1. In some embodiments, the cancer is ovarian cancer or breast cancer, characterized by amplification or overexpression of CCNE1.

In some embodiments, provided herein is a method of treating a disease or disorder associated with CDK2 in a patient, comprising administering to the patient a therapeutically effective amount of the solid form and salts described herein. In some embodiments, the disease or disorder associated with CDK2 is associated with an amplification of the cyclin E1 (CCNE1) gene and/or overexpression of CCNE1.

In some embodiments, the disease or disorder associated with CDK2 is N-myc amplified neuroblastoma cells (see Molenaar, et al., Proc Natl Acad Sci USA 106(31): 12968-12973) K-Ras mutant lung cancers (see Hu, S., et al., Mol Cancer Ther, 2015. 14(11): 2576-85, and cancers with FBW7 mutation and CCNE1 overexpression (see Takada, et al., Cancer Res, 2017. 77(18): 4881-4893).

In some embodiments, the disease or disorder associated with CDK2 is lung squamous cell carcinoma, lung adenocarcinoma, pancreatic adenocarcinoma, breast invasive carcinoma, uterine carcinosarcoma, ovarian serous cystadenocarcinoma, stomach adenocarcinoma, esophageal carcinoma, bladder urothelial carcinoma, mesothelioma, or sarcoma.

In some embodiments, the disease or disorder associated with CDK2 is lung adenocarcinoma, breast invasive carcinoma, uterine carcinosarcoma, ovarian serous cystadenocarcinoma, or stomach adenocarcinoma.

In some embodiments, the disease or disorder associated with CDK2 is an adenocarcinoma, carcinoma, or cystadenocarcinoma.

In some embodiments, the disease or disorder associated with CDK2 is uterine cancer, ovarian cancer, stomach cancer, esophageal cancer, lung cancer, bladder cancer, pancreatic cancer, or breast cancer.

In some embodiments, the disease or disorder associated with CDK2 is a cancer.

In some embodiments, the cancer is characterized by amplification or overexpression of CCNE1. In some embodiments, the cancer is ovarian cancer or breast cancer, characterized by amplification or overexpression of CCNE1.

In some embodiments, the breast cancer is chemotherapy or radiotherapy resistant breast cancer, endocrine resistant breast cancer, trastuzumab resistant breast cancer, or breast cancer demonstrating primary or acquired resistance to CDK4/6 inhibition. In some embodiments, the breast cancer is advanced or metastatic breast cancer.

Examples of cancers that are treatable using the compounds of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers.

In some embodiments, cancers treatable with compounds of the present disclosure include melanoma (e.g., metastatic malignant melanoma, BRAF and HSP90 inhibition-resistant melanoma), renal cancer (e.g., clear cell carcinoma), prostate cancer (e.g., hormone refractory prostate adenocarcinoma), breast cancer, colon cancer, lung cancer (e.g., non-small cell lung cancer and small cell lung cancer), squamous cell head and neck cancer, urothelial cancer (e.g., bladder) and cancers with high microsatellite instability (MSI^{high}) Additionally, the disclosure includes refractory or recurrent malignancies whose growth may be inhibited using the compounds of the disclosure.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including follicular lymphoma, including relapsed or refractory NHL and recurrent follicular), Hodgkin lymphoma or multiple myeloma) and combinations of said cancers.

In some embodiments, cancers that are treatable using the compounds of the present disclosure include, but are not limited to, cholangiocarcinoma, bile duct cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, the compounds of the present disclosure can be used to treat sickle cell disease and sickle cell anemia.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), and essential thrombocytosis (ET)), myelodysplasia syndrome (MDS), T-cell acute lymphoblastic lymphoma (T-ALL) and multiple myeloma (MM).

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC), small cell lung cancer (SCLC), bronchogenic carcinoma, squamous cell, undifferentiated small cell, undifferentiated large cell, adenocarcinoma, alveolar (bronchiolar) carcinoma, bronchial adenoma, chondromatous hamartoma, and mesothelioma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors.

Exemplary nervous system cancers include cancers of the skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, Merkel cell carcinoma, squamous cell carcinoma, Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

It is believed that the solid form and salts described herein may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

The terms “individual,” “patient,” and “subject” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Combination Therapies

### I. Cancer Therapies

Cancer cell growth and survival can be impacted by dysfunction in multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

One or more additional pharmaceutical agents such as, for example, chemotherapeutics, anti-inflammatory agents, steroids, immunosuppressants, immune-oncology agents, metabolic enzyme inhibitors, chemokine receptor inhibitors, and phosphatase inhibitors, as well as targeted therapies such as Bcr-Ab1, Flt-3, EGFR, HER2, JAK, c-MET, VEGFR, PDGFR, c-Kit, IGF-1R, RAF, FAK, and CDK4/6 kinase inhibitors such as, for example, those described in WO 2006/056399 can be used in combination with the compounds of the present disclosure for treatment of CDK2-associated diseases, disorders or conditions. Other agents such as therapeutic antibodies can be used in combination with the compounds of the present disclosure for treatment of CDK2-associated diseases, disorders or conditions. The one or more additional pharmaceutical agents can be administered to a patient simultaneously or sequentially.

In some embodiments, the solid form and salts described herein are administered or used in combination with a BCL2 inhibitor or a CDK4/6 inhibitor.

The compounds as disclosed herein can be used in combination with one or more other enzyme/protein/receptor inhibitors therapies for the treatment of diseases, such as cancer and other diseases or disorders described herein. Examples of diseases and indications treatable with combination therapies include those as described herein. Examples of cancers include solid tumors and non-solid tumors, such as liquid tumors, and blood cancers. Examples of infections include viral infections, bacterial infections, fungus infections or parasite infections. For example, the compounds of the present disclosure can be combined with one or more inhibitors of the following kinases for the treatment of cancer: Akt1, Akt2, Akt3, BCL2, CDK4/6, TGF-βR, PKA, PKG, PKC, CaM-kinase, phosphorylase kinase, MEKK, ERK, MAPK, mTOR, EGFR, HER2, HER3, HER4, INS-R, IDH2, IGF-1R, IR-R, PDGFαR, PDGFβR, PI3K (alpha, beta, gamma, delta, and multiple or selective), CSF1R, KIT, FLK-II, KDR/FLK-1, FLK-4, fit-1, FGFR1, FGFR2, FGFR3, FGFR4, c-Met, PARP, Ron, Sea, TRKA, TRKB, TRKC, TAM kinases (Axl, Mer, Tyro3), FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. In some embodiments, the compounds of the present disclosure can be combined with one or more of the following inhibitors for the treatment of cancer or infections. Non-limiting examples of inhibitors that can be combined with the compounds of the present disclosure for treatment of cancer and infections include an FGFR inhibitor (FGFR1, FGFR2, FGFR3 or FGFR4, e.g., pemigatinib (INCB54828), INCB62079), an EGFR inhibitor (also known as ErB-1 or HER-1; e.g., erlotinib, gefitinib, vandetanib, osimertinib, cetuximab, necitumumab, or panitumumab), a VEGFR inhibitor or pathway blocker (e.g. bevacizumab, pazopanib, sunitinib, sorafenib, axitinib, regorafenib, ponatinib, cabozantinib, vandetanib, ramucirumab, lenvatinib, ziv-aflibercept), a PARP inhibitor (e.g., olaparib, rucaparib, veliparib or niraparib), a JAK inhibitor (JAK1 and/or JAK2, e.g., ruxolitinib or baricitinib; JAK1, e.g., itacitinib (INCB39110), INCB052793, or INCB054707), an IDO inhibitor (e.g., epacadostat, NLG919, or BMS-986205, MK7162), an LSD1 inhibitor (e.g., GSK2979552, INCB59872 and INCB60003), a TDO inhibitor, a PI3K-delta inhibitor (e.g., parsaclisib (INCB50465) or INCB50797), a PI3K-gamma inhibitor such as PI3K-gamma selective inhibitor, a Pim inhibitor (e.g., INCB53914), a CSF1R inhibitor, a TAM receptor tyrosine kinases (Tyro-3, Axl, and Mer; e.g., INCB081776), an adenosine receptor antagonist (e.g., A2a/A2b receptor antagonist), an HPK1 inhibitor, a chemokine receptor inhibitor (e.g., CCR2 or CCR5 inhibitor), a SHP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), c-MET inhibitors (e.g., capmatinib), an anti-CD19 antibody (e.g., tafasitamab), an ALK2 inhibitor (e.g., INCB00928); or combinations thereof.

In some embodiments, the solid form and salts described herein are administered with a PI3Kδ inhibitor. In some embodiments, the compound or salt described herein is administered with a JAK inhibitor. In some embodiments, the solid form and salts described herein are administered with a JAK1 or JAK2 inhibitor (e.g., baricitinib or ruxolitinib). In some embodiments, the solid form and salts described herein are administered with a JAK1 inhibitor. In some embodiments, the solid form and salts described herein are administered with a JAK1 inhibitor, which is selective over JAK2.

Example antibodies for use in combination therapy include, but are not limited to, trastuzumab (e.g., anti-HER2), ranibizumab (e.g., anti-VEGF-A), bevacizumab (AVASTIN™, e.g., anti-VEGF), panitumumab (e.g., anti-EGFR), cetuximab (e.g., anti-EGFR), rituxan (e.g., anti-CD20), and antibodies directed to c-MET.

One or more of the following agents may be used in combination with the solid form and salts described herein and are presented as a non-limiting list: a cytostatic agent, cisplatin, doxorubicin, taxotere, taxol, etoposide, irinotecan, camptosar, topotecan, paclitaxel, docetaxel, epothilones, tamoxifen, 5-fluorouracil, methotrexate, temozolomide, cyclophosphamide, SCH 66336, R115777, L778,123, BMS 214662, IRESSA™ (gefitinib), TARCEVA™ (erlotinib), antibodies to EGFR, intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, leucovirin, ELOXATIN™ (oxaliplatin), pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide 17.alpha.-ethinylestradiol, diethylstilbestrol, testosterone, Prednisone, Fluoxymesterone, Dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, avastin, HERCEPTIN™ (trastuzumab), BEXXAR™ (tositumomab), VELCADE™ (bortezomib), ZEVALIN™ (ibritumomab tiuxetan), TRISENOX™ (arsenic trioxide), XELODA™ (capecitabine), vinorelbine, porfimer, ERBITUX™ (cetuximab), thiotepa, altretamine, melphalan, trastuzumab, lerozole, fulvestrant, exemestane, ifosfomide, rituximab, C225 (cetuximab), Campath (alemtuzumab), clofarabine, cladribine, aphidicolon, rituxan, sunitinib, dasatinib, tezacitabine, Sml1, fludarabine, pentostatin, triapine, didox, trimidox, amidox, 3-AP, and MDL-101,731.

The solid form and salts described herein can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, RIG-I agonists, oncolytic virotherapy and immunomodulating small molecules, including thalidomide or JAK1/2 inhibitor, PI3Kδ inhibitor and the like. The compounds can be administered in combination with one or more anti-cancer drugs, such as a chemotherapeutic agent. Examples of chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

Additional examples of chemotherapeutics include proteasome inhibitors (e.g., bortezomib), thalidomide, revlimid, and DNA-damaging agents such as melphalan, doxorubicin, cyclophosphamide, vincristine, etoposide, carmustine, and the like.

Example steroids include corticosteroids such as dexamethasone or prednisone.

Example Bcr-Ab1 inhibitors include imatinib mesylate (GLEEVAC™), nilotinib, dasatinib, bosutinib, and ponatinib, and pharmaceutically acceptable salts. Other example suitable Bcr-Ab1 inhibitors include the compounds, and pharmaceutically acceptable salts thereof, of the genera and species disclosed in U.S. Pat. No. 5,521,184, WO 04/005281, and U.S. Ser. No. 60/578,491.

Example suitable Flt-3 inhibitors include midostaurin, lestaurtinib, linifanib, sunitinib, sunitinib, maleate, sorafenib, quizartinib, crenolanib, pacritinib, tandutinib, PLX3397 and ASP2215, and their pharmaceutically acceptable salts. Other example suitable Flt-3 inhibitors include compounds, and their pharmaceutically acceptable salts, as disclosed in WO 03/037347, WO 03/099771, and WO 04/046120.

Example suitable RAF inhibitors include dabrafenib, sorafenib, and vemurafenib, and their pharmaceutically acceptable salts. Other example suitable RAF inhibitors include compounds, and their pharmaceutically acceptable salts, as disclosed in WO 00/09495 and WO 05/028444.

Example suitable FAK inhibitors include VS-4718, VS-5095, VS-6062, VS-6063, BI853520, and GSK2256098, and their pharmaceutically acceptable salts. Other example suitable FAK inhibitors include compounds, and their pharmaceutically acceptable salts, as disclosed in WO 04/080980, WO 04/056786, WO 03/024967, WO 01/064655, WO 00/053595, and WO 01/014402.

Example suitable CDK4/6 inhibitors include palbociclib, ribociclib, trilaciclib, lerociclib, and abemaciclib, and their pharmaceutically acceptable salts. Other example suitable CDK4/6 inhibitors include compounds, and their pharmaceutically acceptable salts, as disclosed in WO 09/085185, WO 12/129344, WO 11/101409, WO 03/062236, WO 10/075074, and WO 12/061156.

In some embodiments, the solid form and salts described herein can be used in combination with one or more other kinase inhibitors including imatinib, particularly for treating patients resistant to imatinib or other kinase inhibitors.

In some embodiments, the solid form and salts described herein can be used in combination with a chemotherapeutic in the treatment of cancer, and may improve the treatment response as compared to the response to the chemotherapeutic agent alone, without exacerbation of its toxic effects. In some embodiments, the solid form and salts described herein can be used in combination with a chemotherapeutic provided herein. For example, additional pharmaceutical agents used in the treatment of multiple myeloma, can include, without limitation, melphalan, melphalan plus prednisone [MP], doxorubicin, dexamethasone, and Velcade (bortezomib). Further additional agents used in the treatment of multiple myeloma include Bcr-Ab1, Flt-3, RAF and FAK kinase inhibitors. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM). Additive or synergistic effects are desirable outcomes of combining a CDK2 inhibitor of the present disclosure with an additional agent.

The agents can be combined with solid form and salts described herein in a single or continuous dosage form, or the agents can be administered simultaneously or sequentially as separate dosage forms.

The solid form and salts described herein can be used in combination with one or more other inhibitors or one or more therapies for the treatment of infections. Examples of infections include viral infections, bacterial infections, fungus infections or parasite infections.

In some embodiments, a corticosteroid such as dexamethasone is administered to a patient in combination with the solid form and salts described herein where the dexamethasone is administered intermittently as opposed to continuously.

The solid form and salts described herein can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The solid form and salts described herein can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the compounds of the present disclosure can be used in combination with tumor specific antigen such as heat shock proteins isolated from tumor tissue itself. In some embodiments, the c solid form and salts described herein can be combined with dendritic cells immunization to activate potent anti-tumor responses.

The solid form and salts described herein can be used in combination with bispecific macrocyclic peptides that target Fe alpha or Fe gamma receptor-expressing effectors cells to tumor cells. The solid form and salts described herein can also be combined with macrocyclic peptides that activate host immune responsiveness.

In some further embodiments, combinations of the solid form and salts described herein with other therapeutic agents can be administered to a patient prior to, during, and/or after a bone marrow transplant or stem cell transplant. The solid form and salts described herein can be used in combination with bone marrow transplant for the treatment of a variety of tumors of hematopoietic origin.

The solid form and salts described herein can be used in combination with vaccines, to stimulate the immune response to pathogens, toxins, and self-antigens. Examples of pathogens for which this therapeutic approach may be particularly useful include pathogens for which there is currently no effective vaccine, or pathogens for which conventional vaccines are less than completely effective. These include, but are not limited to, HIV, Hepatitis (A, B, & C), Influenza, Herpes, Giardia, Malaria, Leishmania, Staphylococcus aureus, Pseudomonas Aeruginosa.

Viruses causing infections treatable by methods of the present disclosure include, but are not limited to human papillomavirus, influenza, hepatitis A, B, C or D viruses, adenovirus, poxvirus, herpes simplex viruses, human cytomegalovirus, severe acute respiratory syndrome virus, Ebola virus, measles virus, herpes virus (e.g., VZV, HSV-1, HAV-6, HSV-II, and CMV, Epstein Barr virus), flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Pathogenic bacteria causing infections treatable by methods of the disclosure include, but are not limited to, chlamydia, rickettsial bacteria, mycobacteria, staphylococci, streptococci, pneumococci, meningococci and conococci, klebsiella, proteus, serratia, pseudomonas, legionella, diphtheria, salmonella, bacilli, cholera, tetanus, botulism, anthrax, plague, leptospirosis, and Lyme's disease bacteria.

Pathogenic fungi causing infections treatable by methods of the disclosure include, but are not limited to, Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Pathogenic parasites causing infections treatable by methods of the disclosure include, but are not limited to, Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

When more than one pharmaceutical agent is administered to a patient, they can be administered simultaneously, separately, sequentially, or in combination (e.g., for more than two agents).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, N.J.), the disclosure of which is incorporated herein by reference as if set forth in its entirety.

### II. Immune-Checkpoint Therapies

The solid form and salts described herein can be used in combination with one or more immune checkpoint inhibitors for the treatment of diseases, such as cancer or infections. Exemplary immune checkpoint inhibitors include inhibitors against immune checkpoint molecules such as CBL-B, CD20, CD28, CD40, CD70, CD122, CD96, CD73, CD47, CDK2, GITR, CSF1R, JAK, PI3K delta, PI3K gamma, TAM, arginase, HPK1, CD137 (also known as 4-1BB), ICOS, A2AR, B7-H3, B7-H4, BTLA, CTLA-4, LAG3, TIM3, TLR (TLR7/8), TIGIT, CD112R, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, TIGIT, and VISTA. In some embodiments, the solid form and salts described herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the solid form and salts described herein can be used in combination with one or more agonists of immune checkpoint molecules, e.g., OX40, CD27, GITR, and CD137 (also known as 4-1BB).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1 or PD-L1, e.g., an anti-PD-1 or anti-PD-L1 monoclonal antibody. In some embodiments, the anti-PD-1 or anti-PD-L1 antibody is nivolumab, pembrolizumab, atezolizumab, durvalumab, avelumab, cemiplimab, atezolizumab, avelumab, tislelizumab, spartalizumab (PDR001), cetrelimab (JNJ-63723283), toripalimab (JS001), camrelizumab (SHR-1210), sintilimab (IBI308), AB122 (GLS-010), AMP-224, AMP-514/MEDI-0680, BMS936559, JTX-4014, BGB-108, SHR-1210, MEDI4736, FAZ053, BCD-100, KN035, CS1001, BAT1306, LZM009, AK105, HLX10, SHR-1316, CBT-502 (TQB2450), A167 (KL-A167), STI-A101 (ZKAB001), CK-301, BGB-A333, MSB-2311, HLX20, TSR-042, or LY3300054. In some embodiments, the inhibitor of PD-1 or PD-L1 is one disclosed in U.S. Pat. Nos. 7,488,802, 7,943,743, 8,008,449, 8,168,757, 8,217, 149, WO 03042402, WO 2008156712, WO 2010089411, WO 2010036959, WO 2011066342, WO 2011159877, WO 2011082400, or WO 2011161699, which are each incorporated herein by reference in its entirety.

In some embodiments, the antibody is an anti-PD-1 antibody, e.g., an anti-PD-1 monoclonal antibody. In some embodiments, the anti-PD-1 antibody is nivolumab, pembrolizumab, cemiplimab, spartalizumab, camrelizumab, cetrelimab, toripalimab, sintilimab, AB122, AMP-224, JTX-4014, BGB-108, BCD-100, BAT1306, LZM009, AK105, HLX10, or TSR-042. In some embodiments, the anti-PD-1 antibody is nivolumab, pembrolizumab, cemiplimab, spartalizumab, camrelizumab, cetrelimab, toripalimab, or sintilimab. In some embodiments, the anti-PD-1 antibody is pembrolizumab. In some embodiments, the anti-PD-1 antibody is nivolumab. In some embodiments, the anti-PD-1 antibody is cemiplimab. In some embodiments, the anti-PD-1 antibody is spartalizumab. In some embodiments, the anti-PD-1 antibody is camrelizumab. In some embodiments, the anti-PD-1 antibody is cetrelimab. In some embodiments, the anti-PD-1 antibody is toripalimab. In some embodiments, the anti-PD-1 antibody is sintilimab. In some embodiments, the anti-PD-1 antibody is AB122. In some embodiments, the anti-PD-1 antibody is AMP-224. In some embodiments, the anti-PD-1 antibody is JTX-4014. In some embodiments, the anti-PD-1 antibody is BGB-108. In some embodiments, the anti-PD-1 antibody is BCD-100. In some embodiments, the anti-PD-1 antibody is BAT1306. In some embodiments, the anti-PD-1 antibody is LZM009. In some embodiments, the anti-PD-1 antibody is AK105. In some embodiments, the anti-PD-1 antibody is HLX10. In some embodiments, the anti-PD-1 antibody is TSR-042. In some embodiments, the anti-PD-1 monoclonal antibody is nivolumab or pembrolizumab. In some embodiments, the anti-PD-1 monoclonal antibody is MGA012. In some embodiments, the anti-PD1 antibody is SHR-1210. Other anti-cancer agent(s) include antibody therapeutics such as 4-1BB (e.g., urelumab, utomilumab). In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, avelumab, durvalumab, tislelizumab, BMS-935559, MEDI4736, atezolizumab (MPDL3280A; also known as RG7446), avelumab (MSB0010718C), FAZ053, KN035, CS1001, SHR-1316, CBT-502, A167, STI-A101, CK-301, BGB-A333, MSB-2311, HLX20, or LY3300054. In some embodiments, the anti-PD-L1 antibody is atezolizumab, avelumab, durvalumab, or tislelizumab. In some embodiments, the anti-PD-L1 antibody is atezolizumab. In some embodiments, the anti-PD-L1 antibody is avelumab. In some embodiments, the anti-PD-L1 antibody is durvalumab. In some embodiments, the anti-PD-L1 antibody is tislelizumab. In some embodiments, the anti-PD-L1 antibody is BMS-935559. In some embodiments, the anti-PD-L1 antibody is MEDI4736. In some embodiments, the anti-PD-L1 antibody is FAZ053. In some embodiments, the anti-PD-L1 antibody is KN035. In some embodiments, the anti-PD-L1 antibody is CS1001. In some embodiments, the anti-PD-L1 antibody is SHR-1316. In some embodiments, the anti-PD-L1 antibody is CBT-502. In some embodiments, the anti-PD-L1 antibody is A167. In some embodiments, the anti-PD-L1 antibody is STI-A101. In some embodiments, the anti-PD-L1 antibody is CK-301. In some embodiments, the anti-PD-L1 antibody is BGB-A333. In some embodiments, the anti-PD-L1 antibody is MSB-2311. In some embodiments, the anti-PD-L1 antibody is HLX20. In some embodiments, the anti-PD-L1 antibody is LY3300054.

In some embodiments, the inhibitor of an immune checkpoint molecule is a small molecule that binds to PD-L1, or a pharmaceutically acceptable salt thereof. In some embodiments, the inhibitor of an immune checkpoint molecule is a small molecule that binds to and internalizes PD-L1, or a pharmaceutically acceptable salt thereof. In some embodiments, the inhibitor of an immune checkpoint molecule is a compound selected from those in US 2018/0179201, US 2018/0179197, US 2018/0179179, US 2018/0179202, US 2018/0177784, US 2018/0177870, U.S. Ser. No. 16/369,654 (filed Mar. 29, 2019), and U.S. Ser. No. 62/688,164, or a pharmaceutically acceptable salt thereof, each of which is incorporated herein by reference in its entirety.

In some embodiments, the inhibitor of an immune checkpoint molecule is an inhibitor of KIR, TIGIT, LAIR1, CD160, 2B4 and TGFR beta.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, INCAGN2385, or eftilagimod alpha (IMP321). In some embodiments, the inhibitor of an immune checkpoint molecule is an inhibitor of CD73. In some embodiments, the inhibitor of CD73 is oleclumab.

In some embodiments, the inhibitor of an immune checkpoint molecule is an inhibitor of TIGIT. In some embodiments, the inhibitor of TIGIT is OMP-31M32.

In some embodiments, the inhibitor of an immune checkpoint molecule is an inhibitor of VISTA. In some embodiments, the inhibitor of VISTA is JNJ-61610588 or CA-170.

In some embodiments, the inhibitor of an immune checkpoint molecule is an inhibitor of B7-H3. In some embodiments, the inhibitor of B7-H3 is enoblituzumab, MGD009, or 8H9.

In some embodiments, the inhibitor of an immune checkpoint molecule is an inhibitor of KIR. In some embodiments, the inhibitor of KIR is lirilumab or IPH4102.

In some embodiments, the inhibitor of an immune checkpoint molecule is an inhibitor of A2aR. In some embodiments, the inhibitor of A2aR is CPI-444.

In some embodiments, the inhibitor of an immune checkpoint molecule is an inhibitor of TGF-beta. In some embodiments, the inhibitor of TGF-beta is trabedersen, galusertinib, or M7824.

In some embodiments, the inhibitor of an immune checkpoint molecule is an inhibitor of PI3K-gamma. In some embodiments, the inhibitor of PI3K-gamma is IPI-549.

In some embodiments, the inhibitor of an immune checkpoint molecule is an inhibitor of CD47. In some embodiments, the inhibitor of CD47 is Hu5F9-G4 or TTI-621.

In some embodiments, the inhibitor of an immune checkpoint molecule is an inhibitor of CD73. In some embodiments, the inhibitor of CD73 is MEDI9447.

In some embodiments, the inhibitor of an immune checkpoint molecule is an inhibitor of CD70. In some embodiments, the inhibitor of CD70 is cusatuzumab or BMS-936561.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the agonist of an immune checkpoint molecule is an agonist of OX40, CD27, CD28, GITR, ICOS, CD40, TLR7/8, and CD137 (also known as 4-1BB).

In some embodiments, the agonist of CD137 is urelumab. In some embodiments, the agonist of CD137 is utomilumab.

In some embodiments, the agonist of an immune checkpoint molecule is an inhibitor of GITR. In some embodiments, the agonist of GITR is TRX518, MK-4166, INCAGN1876, MK-1248, AMG228, BMS-986156, GWN323, MEDI1873, or MEDI6469. In some embodiments, the agonist of an immune checkpoint molecule is an agonist of OX40, e.g., OX40 agonist antibody or OX40L fusion protein. In some embodiments, the anti-OX40 antibody is INCAGN01949, MEDI0562 (tavolimab), MOXR-0916, PF-04518600, GSK3174998, BMS-986178, or 9B12. In some embodiments, the OX40L fusion protein is MEDI6383.

In some embodiments, the agonist of an immune checkpoint molecule is an agonist of CD40. In some embodiments, the agonist of CD40 is CP-870893, ADC-1013, CDX-1140, SEA-CD40, R07009789, JNJ-64457107, APX-005M, or Chi Lob 7/4.

In some embodiments, the agonist of an immune checkpoint molecule is an agonist of ICOS. In some embodiments, the agonist of ICOS is GSK-3359609, JTX-2011, or MEDI-570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MEDI9197.

The solid form and salts described herein can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor. In some embodiments, the bispecific antibody binds to PD-1 and PD-L1. In some embodiments, the bispecific antibody that binds to PD-1 and PD-L1 is MCLA-136. In some embodiments, the bispecific antibody binds to PD-L1 and CTLA-4. In some embodiments, the bispecific antibody that binds to PD-L1 and CTLA-4 is AK104.

In some embodiments, the solid form and salts described herein can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

As provided throughout, the additional compounds, inhibitors, agents, etc. can be combined with the present compound in a single or continuous dosage form, or they can be administered simultaneously or sequentially as separate dosage forms.

### Pharmaceutical Formulations and Dosage Forms

When employed as pharmaceuticals, the solid form and salts described herein can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, solid form and salts described herein in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The solid form and salts described herein may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form, each dosage containing from about 5 to about 1000 mg (1 g), or more, such as about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the disclosure contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the disclosure contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the disclosure contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses of the disclosure.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of the solid form and salts described herein. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication, e.g., psoriasis or other skin condition.

The amount of active ingredient or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the solid form and salts described herein can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the solid form and salts described herein can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are listed herein.

### Kits

The present disclosure also includes pharmaceutical kits useful, for example, in the treatment or prevention of CDK2-associated diseases or disorders (such as, e.g., cancer, an inflammatory disease, a cardiovascular disease, or a neurodegenerative disease) which include one or more containers containing a pharmaceutical composition comprising a therapeutically effective amount of the solid form and salts described herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### Biomarkers and Pharmacodynamics Markers

The disclosure further provides predictive markers (e.g., biomarkers and pharmacodynamic markers, e.g., gene copy number, gene sequence, expression levels, or phosphorylation levels) to identify those human subjects having, suspected of having, or at risk of developing a disease or disorder associated with CDK2 for whom administering a CDK2 inhibitor (“a CDK2 inhibitor” as used herein refers to solid form and salts described herein) is likely to be effective. The disclosure also provides pharmacodynamic markers (e.g., phosphorylation levels) to identify those human subjects having, suspected of having, or at risk of developing a disease or disorder associated with CDK2 whom are responding to a CDK2 inhibitor. The use of CCNE1, p16, and Rb S780 is further described in U.S. Patent Publ. No. 2020/0316064), the figures and disclosure of which is incorporated by reference herein in its entirety.

The methods are based, at least in part, on the discovery that the functional status of cyclin dependent kinase inhibitor 2A (“CDKN2A”; also referred to as “p16”) is a biomarker for predicting sensitivity to CDK2-targeting therapies in G1/S-specific cyclin-E1- (“CCNE1-”) amplified cells suitable for use in patient stratification. In addition, the present disclosure is based, at least in part, on the discovery that, in CCNE1-amplified cell lines, the level of human retinoblastoma associated protein (“Rb”) phosphorylation at the serine corresponding to amino acid position 780 of SEQ ID NO:3 is a pharmacodynamic marker for CDK2 activity and is suitable for use in measuring CDK2 enzymatic activity in cellular assay or preclinical and clinical applications, such as, e.g., monitoring the progress of or responsiveness to treatment with a CDK2 inhibitor.

CCNE1 and p16

CCNE1 and p16 have been identified in the Examples as genes, in combination, useful in predicting responsiveness (e.g., improvement in disease as evidenced by disease remission/resolution) of a subject having a disease or disorder associated with CDK2 to a CDK2 inhibitor.

p16 (also known as cyclin-dependent kinase inhibitor 2A, cyclin-dependent kinase 4 inhibitor A, multiple tumor suppressor 1, and p16-INK4a) acts as a negative regulator of the proliferation of normal cells by interacting with CDK4 and CDK6. p16 is encoded by the cyclin dependent kinase inhibitor 2A (“CDKN2A”) gene (GenBank Accession No. NM_000077). The cytogenic location of the CDKN2A gene is 9p21.3, which is the short (p) arm of chromosome 9 at position 21.3. The molecular location of the CDKN2A gene is base pairs 21,967,752 to 21,995,043 on chromosome 9 (Homo sapiens Annotation Release 109, GRCh38.p12). Genetic and epigenetic abnormalities in the gene encoding p16 are believed to lead to escape from senescence and cancer formation (Okamoto et al., 1994, PNAS 91(23):11045-9). Nonlimiting examples of genetic abnormalities in the gene encoding p16 are described in Table A, below. The amino acid sequence of human p16 is provided below (GenBank Accession No. NP_000068/UniProtKB Accession No. P42771):

```
(SEQ ID NO: 1)
    1 MEPAAGSSME PSADWLATAA ARGRVEEVRA
        LLEAGALPNA PNSYGRRPIQ VMMMGSARVA
  61 ELLLLHGAEP NCADPATLTR PVHDAAREGF
        LDTLVVLHRA GARLDVRDAW GRLPVDLAEE
121 LGHRDVARYL RAAAGGTRGS NHARIDAAEG
        PSDIPD.
```

CCNE1 is a cell cycle factor essential for the control of the cell cycle at the G1/S transition (Ohtsubo et al., 1995, Mol. Cell. Biol. 15:2612-2624). CCNE1 acts as a regulatory subunit of CDK2, interacting with CDK2 to form a serine/threonine kinase holoenzyme complex. The CCNE1 subunit of this holoenzyme complex provides the substrate specificity of the complex (Honda et al., 2005, EMBO 24:452-463). CCNE1 is encoded by the cyclin E1 (“CCNE1”) gene (GenBank Accession No. NM_001238). The amino acid sequence of human CCNE1 is provided below (GenBank Accession No. NP_001229/UniProtKB Accession No. P24864):

```
(SEQ ID NO: 2)
    1 mprerrerda kerdtmkedg gaefsarsrk
        rkanvtvflq dpdeemakid rtardqcgsq
  61 pwdnnavcad pcsliptpdk edddrvypns
        tckpriiaps rgsplpvlsw anreevwkim
121 Inkektylrd qhfleqhpll qpkmrailld
        wlmevcevyk Ihretfylaq dffdrymatq
181 envvktllql igisslfiaa kleeiyppkl
        hqfayvtdga csgdeiltme lmimkalkwr
241 1spltivswl nvymqvayln dlhevllpqy
        pqqifiqiae lldlcvldvd clefpygila
301 asalyhfsss elmqkvsgyq wcdiencvkw
        mvpfamvire tgssklkhfr gvadedahni
361 qthrdsldll dkarakkaml seqnrasplp
        sglltppqsg kkqssgpema.
```

The Examples demonstrate CDK2-knockdown inhibits proliferation of CCNE1-amplified cell lines, but not of CCNE1-non-amplified cell lines. Conversely, the Examples show that CDK4/6 inhibition inhibits proliferation of CCNE1-non-amplified cell lines, but not of CCNE1-amplified cell lines. The Examples further demonstrate that presence of a normal (e.g., non-mutated or non-deleted) p16 gene is required for the observed inhibition of cell proliferation in CCNE1-amplified cells treated with a CDK2-inhibitor. Accordingly, CCNE1 and p16 are, together, a combination biomarker: cells that respond to treatment with a CDK2 inhibitor display an amplification of the CCNE1 gene and/or an expression level of CCNE1 that is higher than a control expression level of CCNE1, and have a nucleotide sequence (e.g., a gene or an mRNA) that encodes the p16 protein (e.g., a p16 protein comprising the amino acid sequence of SEQ ID NO:1) and/or have p16 protein present, while control cells that do not respond to treatment with a CDK2 inhibitor do not have an amplification of the CCNE1 gene and/or an expression level of CCNE1 that is higher than a control expression level of CCNE1, and tend to have a mutated or deleted gene that encodes the p16 protein and/or lack expression of p16 protein.

Thus, the disclosure provides a method of treating a human subject having, suspected of having, or at risk of developing a disease or disorder associated with CDK2, comprising administering to the human subject a CDK2 inhibitor, wherein the human subject has been previously determined to: (i) (a) have a nucleotide sequence encoding a p16 protein comprising the amino acid sequence of SEQ ID NO:1, (b) have a CDKN2A gene lacking one or more inactivating nucleic acid substitutions and/or deletions, and/or (c) express a p16 protein, and (ii) (a) have an amplification of the CCNE1 gene and/or (b) have an expression level of CCNE1 in a biological sample obtained from the human subject that is higher than a control expression level of CCNE1. In certain embodiments, the predictive methods described herein predict that the subject will respond to treatment with the CDK2 inhibitor with at least 50%, at least 60%, at least 70%, at least 80%, at least 90%, at least 95%, at least 98% or 100% accuracy. For example, in some embodiments, if the predictive methods described herein are applied to 10 subjects having, suspected of having, or at risk of developing a disease or disorder associated with CDK2, and 8 of those 10 subjects are predicted to respond to treatment with a CDK2 inhibitor based on a predictive method described herein, and 7 of those 8 subjects do indeed respond to treatment with a CDK2 inhibitor, then the predictive method has an accuracy of 87.5% (7 divided by 8). A subject is considered to respond to the CDK2 inhibitor if the subject shows any improvement in disease status as evidenced by, e.g., reduction or alleviation in symptoms, disease remission/resolution, etc.

In some embodiments, the subject has a disease or disorder associated with CDK2. In some embodiments, the human subject has been previously determined to: (i) (a) have a nucleotide sequence encoding a p16 protein comprising the amino acid sequence of SEQ ID NO:1 and/or (b) a CDKN2A gene lacking one or more inactivating nucleic acid substitutions and/or deletions, and (ii) have an amplification of the CCNE1 gene in a biological sample obtained from the human subject. In some embodiments, the CDKN2A gene encodes a protein comprising the amino acid sequence of SEQ ID NO:1. In specific embodiments, the CDKN2A gene encodes a protein comprising the amino acid sequence of SEQ ID NO: 1.

In specific embodiments, the one or more inactivating nucleic acid substitutions and/or deletions in the CDKN2A gene is as described in Table A. In specific embodiments, the one or more inactivating nucleic acid substitutions and/or deletions in the CDKN2A gene is as described in Yarbrough et al., Journal of the National Cancer Institute, 91(18):1569-1574, 1999; Liggett and Sidransky, Biology of Neoplasia, Journal of Oncology, 16(3):1197-1206, 1998, and Cairns et al., Nature Genetics, 11:210-212, 1995, each of which is incorporated by reference herein in its entirety.

TABLE A
CDKN2A gene substitutions, deletions, and modifications

| Description | Reference(s) |
|---|---|
| C to T transition converting codon | RefSNP Accession No. |
| 232 of the CDKN2A gene from an | rs121913388; Kamb et al., |
| arginine codon to a stop codon | Science 264: 436-440, 1994 |
| 19-basepair germline deletion at | RefSNP Accession No. |
| nucleotide 225 causing a reading-frame | rs587776716; Gruis et al., |
| shift predicted to severely truncate p16 protein | Nature Genet. 10: 351-353, 1995 |
| 6-basepair deletion at nucleotides | ClinVar Accession No. |
| 363-368 of the CDKN2A gene | RCV000010017.2; Liu et al., |
| | Oncogene 11: 405-412, 1995 |
| Mutation at chromosome 9: 21971058 predicted to | RefSNP Accession No. |
| substitute glycine corresponding to amino acid | rs104894094; Ciotti et al., |
| position 101 of SEQ ID NO: 1 with a tryptophan | Am. J. Hum. Genet. 67: 311-319, 2000 |
| Germline mutation constituting an | ClinVar Accession No. |
| in-frame 3-basepair duplication at | RCV000010020.3; Borg et al., |
| nucleotide 332 in exon 2 of the CDKN2A gene | Cancer Res. 56: 2497-2500, 1996 |
| Mutation predicted to substitute methionine | RefSNP Accession No. |
| corresponding to amino acid position | rs104894095; Harland et al., |
| 53 of SEQ ID NO: 1 with an isoleucine | Hum. Molec. Genet. 6: 2061-2067, 1997 |
| Mutation predicted to substitute arginine | RefSNP Accession No. |
| corresponding to amino acid position | rs104894097; Monzon et al., |
| 24 of SEQ ID NO: 1 with a proline | New Eng. J. Med. 338: 879-887, 1998 |
| 24-basepair repeat inserted at chromosome 9 | RefSNP Accession No. |
| between 21974795 and 21974796 (forward strand) | rs587780668; Pollock et al., |
| | Hum. Mutat. 11: 424-431, 1998) |
| G-to-T transversion at nucleotide −34 | ClinVar Accession No. |
| of the CDKN2A gene | RCV000010024.5; Liu et al., |
| | Nature Genet. 21: 128-132, 1999 |
| Deletion of the p14(ARF)-specific | ClinVar Accession No. |
| exon 1-beta of CDKN2A | RCV000010026.2; Randerson-Moor et al., |
| | Hum. Molec. Genet. 10: 55-62, 2001 |
| Mutation predicted to substitute valine | RefSNP Accession No. |
| corresponding to amino acid position 126 | rs104894098; Goldstein et al., |
| of SEQ ID NO: 1 with an isoleucine | Brit. J. Cancer 85: 527-530, 2001 |
| Transition (IVS2-105 A-G) in intron 2 of the | ClinVar Accession No. |
| CDKN2A gene creating a false GT splice donor | RCV000010028.3; Harland et al., |
| site 105 bases 5-prime of exon 3 resulting | Hum. Molec. Genet. 10: 2679-2686, 2001 |
| in aberrant splicing of the mRNA | |
| Mutation predicted to result in substitution of | RefSNP Accession No. |
| glycine corresponding to amino acid position 122 | rs113798404; Hewitt et al., |
| of SEQ ID NO: 1 with an arginine | Hum. Molec. Genet. 11: 1273-1279, 2002 |
| Mutation predicted to result in substitution | RefSNP Accession No. |
| of valine corresponding to amino acid | rs113798404; Yakobson et al., |
| position 59 of SEQ ID NO: 1 with an arginine | Melanoma Res. 11: 569-570, 2001 |
| Tandem germline339G-C transversion and a 340C-T | RefSNP Accession Nos. rs113798404 |
| transition in the CDKN2A gene resulting in | and rs104894104; Kannengiesser et al., |
| substitution of proline corresponding to | Genes Chromosomes Cancer 46: 751-760, 2007 |
| amino acid position 114 of SEQ ID NO: 1 with a serine | |
| Mutation predicted to result in substitution | RefSNP Accession No. |
| of serine corresponding to amino acid | rs104894109; Kannengiesser et al., |
| position 56 of SEQ ID NO: 1 with an isoleucine | Genes Chromosomes Cancer 46: 751-760, 2007 |
| Mutation predicted to result in substitution of | RefSNP Accession No. |
| glycine corresponding to amino acid position 89 | rs137854599; Goldstein et al., |
| of SEQ ID NO: 1 with an aspartic acid | J. Med. Genet. 45: 284-289, 2008 |
| Heterozygous A-to-G transition in exon | ClinVar Accession no. |
| 1B of the CDKN2A gene, affecting | RCV000022943.3; Binni et al., Clin. |
| splicing of the p14(ARF) isoform | Genet. 77: 581-586, 2010 |
| Heterozygous 5-bp duplication (19_23dup) in the | ClinVar Accession No. |
| CDKN2A gene, resulting in a frameshift and | RCV000030680.6; Harinck, F., Kluijt et |
| premature termination | al., J. Med. Genet. 49: 362-365, 2012 |
| Mutation predicted to result in substitution of | Yarbrough et al., Journal of the |
| aspartic acid corresponding to amino acid position | National Cancer Institute, 91(18): 1569-1574 |
| 84 of SEQ ID NO: 1 with a valine | |
| Mutation predicted to result in substitution of | Yarbrough et al., Journal of the |
| aspartic acid corresponding to amino acid position | National Cancer Institute, 91(18): 1569-1574 |
| 84 of SEQ ID NO: 1 with a glycine | |
| Mutation predicted to result in substitution of | Yarbrough et al., Journal of the |
| arginine corresponding to amino acid position 87 of | National Cancer Institute, 91(18): 1569-1574 |
| SEQ ID NO: 1 with a proline | |
| Mutation predicted to result in substitution of | Yarbrough et al., Journal of the |
| proline corresponding to amino acid position 48 of | National Cancer Institute, 91(18): 1569-1574 |
| SEQ ID NO: 1 with a leucine | |
| Mutation predicted to result in substitution of | Yarbrough et al., Journal of the |
| aspartic acid corresponding to amino acid position | National Cancer Institute, 91(18): 1569-1574 |
| 74 of SEQ ID NO: 1 with a asparagine | |
| Mutation predicted to result in substitution of | Yarbrough et al., Journal of the |
| arginine corresponding to amino acid position 87 of | National Cancer Institute, 91(18): 1569-1574 |
| SEQ ID NO: 1 with a leucine | |
| Mutation predicted to result in substitution of | Yarbrough et al., Journal of the |
| asparagine corresponding to amino acid position 71 | National Cancer Institute, 91(18): 1569-1574 |
| of SEQ ID NO: 1 with a serine | |
| Mutation predicted to result in substitution of | Yarbrough et al., Journal of the |
| arginine corresponding to amino acid position 80 of | National Cancer Institute, 91(18): 1569-1574 |
| SEQ ID NO: 1 with a leucine | |
| Mutation predicted to result in substitution of | Yarbrough et al., Journal of the |
| histidine corresponding to amino acid position 83 of | National Cancer Institute, 91(18): 1569-1574 |
| SEQ ID NO: 1 with a tyrosine | |

The disclosure also features a method of treating a human subject having, suspected of having, or at risk of developing a disease or disorder associated with CDK2, comprising: (i) identifying, in a biological sample obtained from the human subject: (a) a nucleotide sequence encoding a p16 protein comprising the amino acid sequence of SEQ ID NO:1, (b) a CDKN2A gene lacking one or more inactivating nucleic acid substitutions, and/or (c) the presence of a p16 protein; (ii) identifying, in a biological sample obtained from the human subject: (a) an amplification of the CCNE1 gene and/or (b) an expression level of CCNE1 that is higher than a control expression level of CCNE1; and (iii) administering a CDK2 inhibitor to the human subject. In some embodiments, the subject has a disease or disorder associated with CDK2. In some embodiments, the subject is suspected of having or is at risk of developing a disease or disorder associated with CDK2. In some embodiments, the method comprises: (i) identifying, in a biological sample obtained from the human subject: (a) a nucleotide sequence encoding a p16 protein comprising the amino acid sequence of SEQ ID NO:1, (b) a CDKN2A gene lacking one or more inactivating nucleic acid substitutions and/or deletions, and/or (c) the presence of a p16 protein; (ii) identifying, in a biological sample obtained from the human subject: (a) an amplification of the CCNE1 gene; and (iii) administering a CDK2 inhibitor to the human subject.

The disclosure also features a method of predicting the response of a human subject having, suspected of having, or at risk of developing a disease or disorder associated with CDK2 to a CDK2 inhibitor, comprising: (i) determining, from a biological sample obtained from the human subject: (a) the nucleotide sequence of a CDKN2A gene, (b) the presence of a CDKN2A gene lacking one or more inactivating nucleic acid substitutions and/or deletions, and/or (c) the presence of a p16 protein; and (ii) determining, from a biological sample obtained from the human subject: (a) the copy number of the CCNE1 gene and/or (b) the expression level of CCNE1, wherein (1) (a) the presence of a CDKN2A gene encoding a p16 protein comprising the amino acid sequence of SEQ ID NO:1, (b) the presence of a CDKN2A gene lacking one or more inactivating nucleic acid substitutions and/or deletions, and/or (c) the presence of a p16 protein, and (2) (a) an amplification of the CCNE1 gene and/or (b) an expression level of CCNE1 that is higher than a control expression level of CCNE1, is predictive that the human subject will respond to the CDK2 inhibitor. In some embodiments, the subject has a disease or disorder associated with CDK2. In some embodiments, the subject is suspected of having or is at risk of developing a disease or disorder associated with CDK2. In some embodiments, the method comprises: (i) determining, from a biological sample obtained from the human subject: (a) the nucleotide sequence of a CDKN2A gene and/or (b) the presence of a CDKN2A gene lacking one or more inactivating nucleic acid substitutions and/or deletions; and (ii) determining, from a biological sample obtained from the human subject: (a) the copy number of the CCNE1 gene, wherein (1) (a) the presence of a CDKN2A gene encoding a p16 protein comprising the amino acid sequence of SEQ ID NO:1 and/or (b) the presence of a CDKN2A gene lacking one or more inactivating nucleic acid substitutions and/or deletions, and (2) (a) an amplification of the CCNE1 gene, is predictive that the human subject will respond to the CDK2 inhibitor.

In specific embodiments, the (i) determining of (a) the nucleotide sequence of a CDKN2A gene, (b) the presence of a CDKN2A gene lacking one or more inactivating nucleic acid substitutions and/or deletions, and/or (c) the presence of a p16 protein is performed before (e.g., at least 1 day, at least 2 days, at least 3 days, at least 4 days, at least 5 days, at least 6 days, at least 7 days, at least 2 weeks, at least 3 weeks, or at least 4 weeks, or from 6 hours to 16 hours, from 6 hours to 20 hours, or from 6 hours to 24 hours, from 2 days to 3 days, from 2 days to 4 days, from 2 days to 5 days, from 2 days to 6 days, from 2 days to 7 days, from 1 week to 2 weeks, from 1 week to 3 weeks, or from 1 week to 4 weeks before) administering to the human subject the CDK2 inhibitor. In specific embodiments, the (ii) determining of (a) the copy number of the CCNE1 gene and/or (b) the expression level of CCNE1 in the biological sample obtained from the human subject is performed before (e.g., at least 1 day, at least 2 days, at least 3 days, at least 4 days, at least 5 days, at least 6 days, at least 7 days, at least 2 weeks, at least 3 weeks, or at least 4 weeks, or from 6 hours to 16 hours, from 6 hours to 20 hours, or from 6 hours to 24 hours, from 2 days to 3 days, from 2 days to 4 days, from 2 days to 5 days, from 2 days to 6 days, from 2 days to 7 days, from 1 week to 2 weeks, from 1 week to 3 weeks, or from 1 week to 4 weeks before) administering to the human subject the CDK2 inhibitor.

An amplification of the CCNE1 gene and/or an expression level of CCNE1 that is higher than a control expression level of CCNE1, combined with the presence of a CDKN2A gene encoding a p16 protein comprising the amino acid sequence of SEQ ID NO:1, the presence of a CDKN2A gene lacking one or more inactivating nucleic acid substitutions and/or deletions, and/or the presence of a p16 protein (e.g., a p16 protein comprising the amino acid sequence of SEQ ID NO:1), is indicative/predictive that a human subject having, suspected of having, or at risk of developing a disease or disorder associated with CDK2 will respond to a CDK2 inhibitor.

In some embodiments, the CCNE1 gene is amplified to a gene copy number from 3 to 25. In specific embodiments, the CCNE1 gene is amplified to a gene copy number of at least 3. In specific embodiments, the CCNE1 gene is amplified to a gene copy number of at least 5. In specific embodiments, the CCNE1 gene is amplified to a gene copy number of at least 7. In specific embodiments, the CCNE1 gene is amplified to a gene copy number of at least 10. In specific embodiments, the CCNE1 gene is amplified to a gene copy number of at least 12. In specific embodiments, the CCNE1 gene is amplified to a gene copy number of at least 14. In specific embodiments, the CCNE1 gene is amplified to a gene copy number of at least 21.

In specific embodiments, the expression level of CCNE1 is the level of CCNE1 mRNA. In specific embodiments, the expression level of CCNE1 is the level of CCNE1 protein.

In some embodiments of the foregoing methods, the control expression level of CCNE1 is a pre-established cut-off value. In some embodiments of the foregoing methods, the control expression level of CCNE1 is the expression level of CCNE1 in a sample or samples obtained from one or more subjects that have not responded to treatment with the CDK2 inhibitor.

In some embodiments of the foregoing methods, the expression level of CCNE1 is the expression level of CCNE1 mRNA. In some embodiments of the foregoing methods, the expression level of CCNE1 is the expression level of CCNE1 protein. In some embodiments in which the expression level of CCNE1 is the expression level of CCNE1 mRNA, the expression level of CCNE1 is measured by RNA sequencing, quantitative polymerase chain reaction (PCR), in situ hybridization, nucleic acid array or RNA sequencing. In some embodiments in which the expression level of CCNE1 is the expression level of CCNE1 protein, the expression level of CCNE1 is measured by western blot, enzyme-linked immunosorbent assay, or immunohistochemistry staining.

Rb S780 The disclosure also features a method for assessing the CDKN2A gene and the CCNE1 gene, comprising determining, from a biological sample or biological samples obtained from a human subject having a disease or disorder associated with CDK2, (i) (a) the nucleotide sequence of a CDKN2A gene or (b) the presence of a CDKN2A gene lacking one or more inactivating nucleic acid substitutions and/or deletions, and (ii) the copy number of the CCNE1 gene.

The disclosure also features a method of evaluating the response of a human subject having, suspected of having, or at risk of developing a disease or disorder associated with CDK2 to a CDK2 inhibitor, comprising: (a) administering a CDK2 inhibitor to the human subject, wherein the human subject has been previously determined to have an amplification of the CCNE1 gene and/or an expression level of CCNE1 that is higher than a control expression level of CCNE1; (b) measuring, in a biological sample of obtained from the subject subsequent to the administering of step (a), the level of retinoblastoma (Rb) protein phosphorylation at the serine corresponding to amino acid position 780 of SEQ ID NO:3, wherein a reduced level of Rb phosphorylation at the serine corresponding to amino acid position 780 of SEQ ID NO:3, as compared to a control level of Rb phosphorylation at the serine corresponding to amino acid position 780 of SEQ ID NO:3, is indicative that the human subject responds to the CDK2 inhibitor. In some embodiments, the subject has a disease or disorder associated with CDK2. In some embodiments, the subject is suspected of having or is at risk of developing a disease or disorder associated with CDK2. In some embodiments, the biological sample comprises a blood sample or a tumor biopsy sample.

Phosphorylation of Rb at the serine corresponding to amino acid position 780 of SEQ ID NO:3 (referred to herein as “Ser780” or “S780”) has been identified in the Examples as a pharmacodynamic marker useful in assessing responsiveness (e.g., inhibition by CDK2) of a human subject having a disease or disorder having CCNE1 amplification to a CDK2 inhibitor.

Rb is a regulator of the cell cycle and acts as a tumor suppressor. Rb is activated upon phosphorylation by cyclin D-CDK4/6 at Ser780 and Ser795 and by cyclin E/CDK2 at Ser807 and Ser811. Rb is encoded by the RB transcriptional corepressor 1 (“RB1”) gene (GenBank Accession No. NM_000321). The amino acid sequence of human Rb is provided below (GenBank Accession No. NP_000312/UniProtKB Accession No. P06400) (S780 is in bold and underlined):

| | (SEQ ID NO: 3) |
|---|---|
| 1 | MPPKTPRKTA ATAAAAAAEP PAPPPPPPPE |
| | EDPEQDSGPE DLPLVRLEFE ETEEPDFTAL |
| 61 | CQKLKIPDHV RERAWLTWEK VSSVDGVLGG |
| | YIQKKKELWG ICIFIAAVDL DEMSFTFTEL |
| 121 | QKNIEISVHK FFNLLKEIDT STKVDNAMSR |
| | LLKKYDVLFA LFSKLERTCE LIYLTQPSSS |
| 181 | ISTEINSALV LKVSWITFLL AKGEVLQMED |
| | DLVISFQLML CVLDYFIKLS PPMLLKEPYK |
| 241 | TAVIPINGSP RTPRRGQNRS ARIAKQLEND |
| | TRIIEVLCKE HECNIDEVKN VYFKNFIPFM |
| 301 | NSLGLVTSNG LPEVENLSKR YEEIYLKNKD |
| | LDARLFLDHD KTLQTDSIDS FETQRTPRKS |
| 361 | NLDEEVNVIP PHTPVRTVMN TIQQLMMILN |
| | SASDOPSENL ISYFNNCTVN PKESILKRVK |
| 421 | DIGYIFKEKF AKAVGOGCVE IGSQRYKLGV |
| | RLYYRVMESM LKSEEERLSI QNFSKLLNDN |
| 481 | IFHMSLLACA LEVVMATYSR STSQNLDSGT |
| | DLSFPWILNV LNLKAFDFYK VIESFIKAEG |
| 541 | NLTREMIKHL ERCEHRIMES LAWLSDSPLF |
| | DLIKQSKDRE GPTDHLESAC PLNLPLQNNH |
| 601 | TAADMYLSPV RSPKKKGSTT RVNSTANAET |
| | QATSAFQTQK PLKSTSLSLF YKKVYRLAYL |
| 661 | RLNTLCERLL SEHPELEHII WTLFQHTLQN |
| | EYELMRDRHL DQIMMCSMYG ICKVKNIDLK |
| 721 | FKIIVTAYKD LPHAVQETFK RVLIKEEEYD |
| | SIIVFYNSVF MQRLKTNILQ YASTRPPTLS |
| 781 | PIPHIPRSPY KFPSSPLRIP GGNIYISPLK |
| | SPYKISEGLP TPTKMTPRSR ILVSIGESFG |
| 841 | TSEKFQKINQ MVCNSDRVLK RSAEGSNPPK |
| | PLKKLRFDIE GSDEADGSKH LPGESKFQQK |
| 901 | LAEMTSTRTR MQKQKMNDSM DTSNKEEK. |

As stated above, the Examples demonstrate CDK2-knockdown inhibits proliferation in CCNE1-amplified cell lines, but not in CCNE1-non-amplified cell lines. The Examples further demonstrate CDK2-knockdown or inhibition blocks Rb phosphorylation at the S780 in CCNE1-amplified cell lines, but not in CCNE1-non-amplified cell lines. Accordingly, Rb phosphorylation at the serine corresponding to amino acid position 780 of SEQ ID NO:3 is a pharmacodynamic marker for assessing response to CDK2 inhibition in CCNE1 amplified cancer cells or patients with diseases or disorders having CCNE1 amplification. Thus, provided herein are methods relating to the use of the level of Rb phosphorylation at the serine corresponding to amino acid position 780 of SEQ ID NO:3 in a human subject having, suspected of having, or at risk of developing a disease or disorder associated with CDK2 as a marker for indicating the response of the human subject to a CDK2 inhibitor, wherein the human subject has an increased expression level of CCNE1.

Thus, the disclosure features a method for measuring the amount of a protein in a sample, comprising: (a) providing a biological sample obtained from a human subject having a disease or disorder associated with CDK2; and (b) measuring the level of Rb protein phosphorylation at the serine corresponding to amino acid position 780 of SEQ ID NO:3 in the biological sample. In some embodiments, the biological sample comprises a blood sample or a tumor biopsy sample. In a specific embodiment, provided herein is a method of evaluating the response of a human subject having, suspected of having, or at risk of developing a disease or disorder associated with CDK2 to a CDK2 inhibitor, comprising: (a) administering a CDK2 inhibitor to the human subject, wherein the human subject has been previously determined to have an amplification of the CCNE1 gene and/or an expression level of CCNE1 that is higher than a control expression level of CCNE1; and (b) measuring, in a biological sample obtained from the human subject subsequent to the administering of step (a), the level of Rb phosphorylation at the serine corresponding to amino acid position 780 of SEQ ID NO:3, wherein a reduced level of Rb phosphorylation at the serine corresponding to amino acid position 780 of SEQ ID NO:3, as compared to a control level of Rb phosphorylation at the serine corresponding to amino acid position 780 of SEQ ID NO:3, is indicative that the human subject responds to the CDK2 inhibitor. In specific embodiments, the human subject has a disease or disorder associated with CDK2.

A reduced level of Rb phosphorylation at the serine corresponding to amino acid position 780 of SEQ ID NO:3, as compared to a control level of Rb phosphorylation at the serine corresponding to amino acid position 780 of SEQ ID NO:3, combined with an amplification of the CCNE1 gene and/or an expression level of CCNE1 that is higher than a control expression level of CCNE1, is indicative that a human subject having, suspected of having, or at risk of developing a disease or disorder associated with CDK2 responds to a CDK2 inhibitor. For example, in a subject having an amplification of the CCNE1 gene and/or an expression level of CCNE1 that is higher than a control expression level of CCNE1, a biological sample, obtained from the subject after treatment with a CDK2 inhibitor, having low (e.g., reduced as compared to a control) or undetectable levels of Rb phosphorylation at serine corresponding to amino acid position 780 of SEQ ID NO:3 is indicative that the subject responds to the CDK2 inhibitor.

A biological sample, obtained from a subject after administration of a CDK2 inhibitor to the subject, having a reduced level of Rb phosphorylation at the serine corresponding to amino acid position 780 of SEQ ID NO:3, as compared to a control level of Rb phosphorylation at the serine corresponding to amino acid position 780 of SEQ ID NO:3, combined with: (i) an amplification of the CCNE1 gene and/or an expression level of CCNE1 that is higher than a control expression level of CCNE1, and (ii) presence of a CDKN2A gene encoding a p16 protein comprising the amino acid sequence of SEQ ID NO:1, presence of a CDKN2A gene lacking one or more inactivating nucleic acid substitutions and/or deletions, and/or presence of a p16 protein (e.g., a p16 protein comprising the amino acid sequence of SEQ ID NO:1), is indicative that a human subject having, suspected of having, or at risk of developing a disease or disorder associated with CDK2 responds to a CDK2 inhibitor. For example, in a human subject having (i) an amplification of the CCNE1 gene and/or an expression level of CCNE1 that is higher than a control expression level of CCNE1, and (ii) the presence of a CDKN2A gene encoding a p16 protein comprising the amino acid sequence of SEQ ID NO:1, the presence of a CDKN2A gene lacking one or more inactivating nucleic acid substitutions and/or deletions, and/or the presence of a p16 protein (e.g., a p16 protein comprising the amino acid sequence of SEQ ID NO:1), a biological sample, obtained from the human subject after administration of a CDK2 inhibitor to the subject, having low (e.g., reduced as compared to a control) or undetectable levels of Rb phosphorylation at the serine corresponding to amino acid position 780 of SEQ ID NO:3 is indicative that the human subject responds to the CDK2 inhibitor.

In some embodiments, the CCNE1 gene is amplified to a gene copy number from 3 to 25. In specific embodiments, the CCNE1 gene is amplified to a gene copy number of at least 3. In specific embodiments, the CCNE1 gene is amplified to a gene copy number of at least 5. In specific embodiments, the CCNE1 gene is amplified to a gene copy number of at least 7. In specific embodiments, the CCNE1 gene is amplified to a gene copy number of at least 10. In specific embodiments, the CCNE1 gene is amplified to a gene copy number of at least 12. In specific embodiments, the CCNE1 gene is amplified to a gene copy number of at least 14. In specific embodiments, the CCNE1 gene is amplified to a gene copy number of at least 21. In specific embodiments, the expression level of CCNE1 is the level of CCNE1 mRNA. In specific embodiments, the expression level of CCNE1 is the level of CCNE1 protein.

Controls

As described above, the methods related to biomarkers and pharmacodynamic markers can involve, measuring one or more markers (e.g., a biomarker or a pharmacodynamics marker, e.g., the amplification of the CCNE1 gene, the expression level of CCNE1, the presence of a CDKN2A gene encoding a p16 protein comprising the amino acid sequence of SEQ ID NO:1, the presence of a CDKN2A gene lacking one or more inactivating nucleic acid substitutions and/or deletions, the presence of a p16 protein (e.g., a p16 protein comprising the amino acid sequence of SEQ ID NO:1), and Rb phosphorylation at the serine corresponding to amino acid position 780 of SEQ ID NO:3) in a biological sample from a human subject having, suspected of having or at risk of developing a disease or disorder associated with CDK2. In specific embodiments, the human subject has a disease or disorder associated with CDK2. In specific embodiments, the human subject is suspected of having or is at risk of developing a disease or disorder associated with CDK2. In certain aspects, the level (e.g., amplification (e.g., for the CCNE1 gene), expression level (e.g., for CCNE1 or p16 protein), or phosphorylation level (e.g., for Rb)) of one or more biomarkers, compared to a control level of the one or more biomarkers, predicts/indicates the response of a human subject to treatment comprising a CDK2 inhibitor. In certain embodiments, when (i) the CCNE1 gene is amplified and/or an expression level of CCNE1 that is higher than a control expression level of CCNE1, and (ii) a CDKN2A gene encoding a p16 protein comprising the amino acid sequence of SEQ ID NO:1 is present, a CDKN2A gene lacking one or more inactivating nucleic acid substitutions and/or deletions is present, and/or a p16 protein (e.g., a p16 protein comprising the amino acid sequence of SEQ ID NO:1) is present, the human subject is identified as likely to respond to a CDK2 inhibitor. In other embodiments, when (i) the CCNE1 gene is amplified and/or an expression level of CCNE1 that is higher than a control expression level of CCNE1, and (ii) in a biological sample from the human subject after the human subject has been administered a CDK2 inhibitor, the level of Rb phosphorylation at the serine corresponding to amino acid position 780 of SEQ ID NO:3 is less than the control level of Rb phosphorylation at the serine corresponding to amino acid position 780 of SEQ ID NO:3, the human subject is identified as responding to a CDK2 inhibitor. In yet another embodiment, when (i) the CCNE1 gene is amplified and/or an expression level of CCNE1 that is higher than a control expression level of CCNE1, (ii) a CDKN2A gene encoding a p16 protein comprising the amino acid sequence of SEQ ID NO:1 is present, a CDKN2A gene lacking one or more inactivating nucleic acid substitutions and/or deletions is present, and/or a p16 protein (e.g., a p16 protein comprising the amino acid sequence of SEQ ID NO:1) is present, and (iii) in a biological sample from the human subject after the human subject has been administered a CDK2 inhibitor, the level of Rb phosphorylation at the serine corresponding to amino acid position 780 of SEQ ID NO:3 is less than the control level of Rb phosphorylation at the serine corresponding to amino acid position 780 of SEQ ID NO:3, the human subject is identified as responding to a CDK2 inhibitor. In this context, the term “control” includes a sample (from the same tissue type) obtained from a human subject who is known to not respond to a CDK2 inhibitor. The term “control” also includes a sample (from the same tissue type) obtained in the past from a human subject who is known to not respond to a CDK2 inhibitor and used as a reference for future comparisons to test samples taken from human subjects for which therapeutic responsiveness is to be predicted. The “control” level (e.g., gene copy number, expression level, or phosphorylation level) for a particular biomarker (e.g., CCNE1, p16, or Rb phosphorylation) in a particular cell type or tissue may be pre-established by an analysis of biomarker level (e.g., expression level or phosphorylation level) in one or more (e.g., 2, 3, 4, 5, 6, 7, 8, 9, 10, 15, 20, 25, 30, 35, or 40 or more) human subjects that have not responded to treatment with a CDK2 inhibitor. This pre-established reference value (which may be an average or median level (e.g., gene copy number, expression level, or phosphorylation level) taken from multiple human subjects that have not responded to the therapy) may then be used for the “control” level of the biomarker (e.g., CCNE1, p16, or Rb phosphorylation) in the comparison with the test sample. In such a comparison, the human subject is predicted to respond to a CDK2 inhibitor if the CCNE1 gene is amplified and/or the expression level of CCNE is higher than the pre-established reference, and a CDKN2A gene encoding a p16 protein comprising the amino acid sequence of SEQ ID NO:1 is present, a CDKN2A gene lacking one or more inactivating nucleic acid substitutions and/or deletions is present, and/or a p16 protein (e.g., a p16 protein comprising the amino acid sequence of SEQ ID NO:1) is present. In another such a comparison, the human subject is predicted to respond to a CDK2 inhibitor if (i) CCNE1 gene is amplified and/or the expression level of CCNE is higher than the pre-established reference, and (ii) after administering to the human subject a CDK2 inhibitor, the level of Rb phosphorylation at the serine corresponding to amino acid position 780 of SEQ ID NO:3 is lower than the pre-established reference. In yet another such a comparison, the human subject is indicated to respond to a CDK2 inhibitor if (i) CCNE1 gene is amplified and/or the expression level of CCNE is higher than the pre-established reference, (ii) a CDKN2A gene encoding a p16 protein comprising the amino acid sequence of SEQ ID NO:1 is present, a CDKN2A gene lacking one or more inactivating nucleic acid substitutions and/or deletions is present, and/or a p16 protein (e.g., a p16 protein comprising the amino acid sequence of SEQ ID NO:1) is present, and (iii) after administering to the human subject a CDK2 inhibitor, the level of Rb phosphorylation at the serine corresponding to amino acid position 780 of SEQ ID NO:3 is lower than the pre-established reference.

The “control” level for a particular biomarker in a particular cell type or tissue may alternatively be pre-established by an analysis of biomarker level in one or more human subjects that have responded to treatment with a CDK2 inhibitor. This pre-established reference value (which may be an average or median level (e.g., expression level or phosphorylation level) taken from multiple human subjects that have responded to the therapy) may then be used as the “control” level (e.g., expression level or phosphorylation level) in the comparison with the test sample. In such a comparison, the human subject is indicated to respond to a CDK2 inhibitor if the level (e.g., copy number of the CCNE1 gene, expression level of CCNE1, expression level of p16, or phosphorylation level of Rb at the serine corresponding to amino acid position 780 of SEQ ID NO:3) of the biomarker being analyzed is equal or comparable to (e.g., at least 85% but less than 115% of), the pre-established reference.

In certain embodiments, the “control” is a pre-established cut-off value. A cut-off value is typically a level (e.g., a copy number, an expression level, or a phosphorylation level) of a biomarker above or below which is considered predictive of responsiveness of a human subject to a therapy of interest. Thus, in accordance with the methods and compositions described herein, a reference level (e.g., of CCNE1 gene copy number, CCNE1 expression, p16 expression, or Rb phosphorylation at the serine corresponding to amino acid position 780 of SEQ ID NO:3) is identified as a cut-off value, above or below of which is predictive of responsiveness to a CDK2 inhibitor. Cut-off values determined for use in the methods described herein can be compared with, e.g., published ranges of concentrations but can be individualized to the methodology used and patient population.

In some embodiments, the expression level of CCNE1 is increased as compared to the expression level of CCNE1 in a control. For example, the expression level of CCNE1 analyzed can be at least 1.5, at least 2, at least 3, at least 4, at least 5, at least 6, at least 7, at least 8, at least 9, at least 10, at least 20, at least 25, at least 50, at least 75, or at least 100 times higher, or at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1,000%, at least 1,500%, at least 2,000%, at least 2,500%, at least 3,000%, at least 3,500%, at least 4,000%, at least 4,500%, or at least 5,000% higher, than the expression level of CCNE1 in a control.

A p16 protein is present if the protein is detectable by any assay known in the art or described herein, such as, for example, western blot, immunohistochemistry, fluorescence-activated cell sorting, and enzyme-linked immunoassay. In some embodiments, a p16 protein is present at an expression level that is within at least 5%, at least 10%, at least 20%, or at least 30% of the p16 expression level in a healthy control.

In some embodiments, the level of Rb phosphorylation at the serine corresponding to amino acid position 780 of SEQ ID NO:3 being analyzed is reduced as compared to the level of Rb phosphorylation at the serine corresponding to amino acid position 780 of SEQ ID NO:3 in a control. For example, the level of the Rb phosphorylation at the serine corresponding to amino acid position 780 of SEQ ID NO:3 being analyzed can be at least 1.5, at least 2, at least 3, at least 4, at least 5, at least 6, at least 7, at least 8, at least 9, at least 10, at least 20, at least 25, at least 50, at least 75, or at least 100 times lower, or at least 10%, at least 20%, at least 30%, at least 40%, at least 50%, at least 60%, at least 70%, at least 80%, at least 90%, or 100% lower, than the level of Rb phosphorylation at the serine corresponding to amino acid position 780 of SEQ ID NO:3 in a control.

Biological Samples

Suitable biological samples for the methods described herein include any sample that contains blood or tumor cells obtained or derived from the human subject in need of treatment. For example, a biological sample can contain tumor cells from biopsy from a patient suffering from a solid tumor. A tumor biopsy can be obtained by a variety of means known in the art. Alternatively, a blood sample can be obtained from a patients suffering from a hematological cancer.

A biological sample can be obtained from a human subject having, suspected of having, or at risk of developing, a disease or disorder associated with CDK2. In some embodiments, the disease or disorder associated with CDK2 is a cancer (such as those described supra).

Methods for obtaining and/or storing samples that preserve the activity or integrity of molecules (e.g., nucleic acids or proteins) in the sample are well known to those skilled in the art. For example, a biological sample can be further contacted with one or more additional agents such as buffers and/or inhibitors, including one or more of nuclease, protease, and phosphatase inhibitors, which preserve or minimize changes in the molecules in the sample.

Evaluating Biomarkers and Pharmacodynamic Markers

Expression levels of CCNE1 or p16 can be detected as, e.g., RNA expression of a target gene (i.e., the genes encoding CCNE1 or p16). That is, the expression level (amount) of CCNE1 or p16 can be determined by detecting and/or measuring the level of mRNA expression of the gene encoding CCNE1. Alternatively, expression levels of CCNE1 or p16 can be detected as, e.g., protein expression of target gene (i.e., the genes encoding CCNE1 or p16). That is, the expression level (amount) of CCNE1 or p16 can be determined by detecting and/or measuring the level of protein expression of the genes encoding CCNE1 or p16.

In some embodiments, the expression level of CCNE1 or p16 is determined by measuring RNA levels. A variety of suitable methods can be employed to detect and/or measure the level of mRNA expression of a gene. For example, mRNA expression can be determined using Northern blot or dot blot analysis, reverse transcriptase-PCR (RT-PCR; e.g., quantitative RT-PCR), in situ hybridization (e.g., quantitative in situ hybridization), nucleic acid array (e.g., oligonucleotide arrays or gene chips) and RNA sequencing analysis. Details of such methods are described below and in, e.g., Sambrook et al., Molecular Cloning: A Laboratory Manual Second Edition vol. 1, 2 and 3. Cold Spring Harbor Laboratory Press: Cold Spring Harbor, N.Y., USA, November 1989; Gibson et al. (1999) Genome Res., 6(10):995-1001; and Zhang et al. (2005) Environ. Sci. Technol., 39(8):2777-2785; U.S. Publication No. 2004086915; European Patent No. 0543942; and U.S. Pat. No. 7,101,663; Kukurba et al. (2015) Cold Spring Harbor Protocols, 2015 (11): 951-69; the disclosures of each of which are incorporated herein by reference in their entirety.

In one example, the presence or amount of one or more discrete mRNA populations in a biological sample can be determined by isolating total mRNA from the biological sample (see, e.g., Sambrook et al. (supra) and U.S. Pat. No. 6,812,341) and subjecting the isolated mRNA to agarose gel electrophoresis to separate the mRNA by size. The size-separated mRNAs are then transferred (e.g., by diffusion) to a solid support such as a nitrocellulose membrane. The presence or amount of one or more mRNA populations in the biological sample can then be determined using one or more detectably-labeled-polynucleotide probes, complementary to the mRNA sequence of interest, which bind to and thus render detectable their corresponding mRNA populations. Detectable-labels include, e.g., fluorescent (e.g., umbelliferone, fluorescein, fluorescein isothiocyanate, rhodamine, dichlorotriazinylamine fluorescein, dansyl chloride, allophycocyanin, or phycoerythrin), luminescent (e.g., europium, terbium, Qdot™ nanoparticles supplied by the Quantum Dot Corporation, Palo Alto, Calif.), radiological (e.g., ¹²⁵I, ¹³¹I, ³⁵S, ³²P, ³³P, or ³H), and enzymatic (horseradish peroxidase, alkaline phosphatase, beta-galactosidase, or acetylcholinesterase) labels.

In some embodiments, the expression level of CCNE1 or p16 is determined by measuring protein levels. A variety of suitable methods can be employed to detect and/or measure the level of protein expression of target genes. For example, CCNE1 or p16 protein expression can be determined using western blot, enzyme-linked immunosorbent assay (“ELISA”), fluorescence activated cell sorting, or immunohistochemistry analysis (e.g., using a CCNE1-specific or p16-specific antibody, respectively). Details of such methods are described below and in, e.g., Sambrook et al., supra.

In one example, the presence or amount of one or more discrete protein populations (e.g., CCNE1 or p16) in a biological sample can be determined by western blot analysis, e.g., by isolating total protein from the biological sample (see, e.g., Sambrook et al. (supra)) and subjecting the isolated protein to agarose gel electrophoresis to separate the protein by size. The size-separated proteins are then transferred (e.g., by diffusion) to a solid support such as a nitrocellulose membrane. The presence or amount of one or more protein populations in the biological sample can then be determined using one or more antibody probes, e.g., a first antibody specific for the protein of interest (e.g., CCNE1 or p16), and a second antibody, detectably labeled, specific for the first antibody, which binds to and thus renders detectable the corresponding protein population. Detectable-labels suitable for use in western blot analysis are known in the art.

Methods for detecting or measuring gene expression (e.g., mRNA or protein expression) can optionally be performed in formats that allow for rapid preparation, processing, and analysis of multiple samples. This can be, for example, in multi-welled assay plates (e.g., 96 wells or 386 wells) or arrays (e.g., nucleic acid chips or protein chips). Stock solutions for various reagents can be provided manually or robotically, and subsequent sample preparation (e.g., RT-PCR, labeling, or cell fixation), pipetting, diluting, mixing, distribution, washing, incubating (e.g., hybridization), sample readout, data collection (optical data) and/or analysis (computer aided image analysis) can be done robotically using commercially available analysis software, robotics, and detection instrumentation capable of detecting the signal generated from the assay. Examples of such detectors include, but are not limited to, spectrophotometers, luminometers, fluorimeters, and devices that measure radioisotope decay. Exemplary high-throughput cell-based assays (e.g., detecting the presence or level of a target protein in a cell) can utilize ArrayScan® VTI HCS Reader or KineticScan® HCS Reader technology (Cellomics Inc., Pittsburgh, Pa.).

In some embodiments, the presence of a CDKN2A gene encoding a p16 protein comprising the amino acid sequence of SEQ ID NO:1 and/or the presence of a CDKN2A gene lacking one or more inactivating nucleic acid substitutions and/or deletions is determined by evaluating the DNA sequence of the CDKN2A gene (e.g., genomic DNA or cDNA) or by evaluating the RNA sequence of the CDKN2A gene (e.g., RNA, e.g., mRNA). Methods of performing nucleic acid sequencing analyses are known in the art and described above. Nonlimiting examples of inactivating nucleic acid substitutions and/or deletions preventing the CDKN2A gene from encoding a protein comprising the amino acid sequence of SEQ ID NO:1 are described in Table A, above. In specific embodiments, the one or more inactivating nucleic acid substitutions and/or deletions in the CDKN2A gene is as described in Yarbrough et al., Journal of the National Cancer Institute, 91(18):1569-1574, 1999; Liggett and Sidransky, Biology of Neoplasia, Journal of Oncology, 16(3):1197-1206, 1998, and Cairns et al., Nature Genetics, 11:210-212, 1995, each of which is incorporated by reference herein in its entirety.

In some embodiments, the expression level of a gene or the presence of a gene lacking one or more inactivating nucleic acid substitutions or deletions is determined by evaluating the copy number variation (CNV) of the gene. The CNV of genes (e.g., the CCNE1 gene and/or the CDKN2A gene) can be determined/identified by a variety of suitable methods. For example, CNV can be determined using fluorescent in situ hybridization (FISH), multiplex ligation dependent probe amplification (MLPA), array comparative genomic hybridization (aCGH), single-nucleotide polymorphisms (SNP) array, and next-generation sequencing (NGS) technologies.

In one example, the copy number variation of one or more discrete genes in a biological sample can be determined by MLPA, e.g., by extracting DNA specimens from the biological sample (see, e.g., Sambrook et al. (supra) and U.S. Pat. No. 6,812,341), and amplifying DNA sequence of interest (e.g., CCNE1 or CDKN2A) using a mixture of MLPA probes. Each MLPA probe consists of two oligonucleotides that hybridize to immediately adjacent target DNA sequence (e.g., CCNE1 or CDKN2A) in order to be ligated into a single probe. Ligated probes are amplified though PCR with one PCR primer fluorescently labeled, enabling the amplification products to be visualized during fragment separation by capillary electrophoresis. The presence, absence or amplification of one or more genes of interest in the biological sample is calculated by measuring PCR derived fluorescence, quantifying the amount of PCR product after normalization and comparing it with control DNA samples.

The level of Rb phosphorylation at the serine corresponding to amino acid position 780 of SEQ ID NO:3 can be detected by a variety of suitable methods. For example, phosphorylation status can be determined using western blot, ELISA, fluorescence activated cell sorting, or immunohistochemistry analysis. Details of such methods are described below and in, e.g., Sambrook et al., supra.

As with the methods for detecting or measuring gene expression (above), methods for detecting or measuring the level of Rb phosphorylation at the serine corresponding to amino acid position 780 of SEQ ID NO:3 can optionally be performed in formats that allow for rapid preparation, processing, and analysis of multiple samples.

### EMBODIMENTS

1. A solid form of a compound of Formula (I):

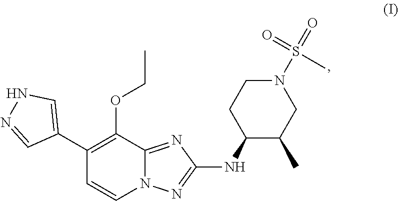 [SMILES: CCOc1c(-c2cn[nH]c2)ccn2nc(N[C@H]3CCN(S(C)(=O)=O)C[C@H]3C)nc12]

which is Form I.
2. The solid form of embodiment 1, which is non-solvated.
3. The solid form of embodiment 1, which is crystalline.
4. The solid form of embodiment 1, wherein the form has at least one XRPD peak, in terms of 2-theta (±0.2 degrees), selected from 7.3, 10.5, 12.8, 14.5, 15.2, 16.4, 20.3, 21.3, 21.6, and 27.0.
5. The solid form of embodiment 1, wherein the form has at least two XRPD peaks, in terms of 2-theta (±0.2 degrees), selected from 7.3, 10.5, 12.8, 14.5, 15.2, 16.4, 20.3, 21.3, 21.6, and 27.0.
6. The solid form of embodiment 1, wherein the form has at least three XRPD peaks, in terms of 2-theta (±0.2 degrees), selected from 7.3, 10.5, 12.8, 14.5, 15.2, 16.4, 20.3, 21.3, 21.6, and 27.0.
7. The solid form of embodiment 1, wherein the form has at least four XRPD peaks, in terms of 2-theta (±0.2 degrees), selected from 7.3, 10.5, 12.8, 14.5, 15.2, 16.4, 20.3, 21.3, 21.6, and 27.0.
8. The solid form of embodiment 1, wherein the form has at least five XRPD peaks, in terms of 2-theta (±0.2 degrees), selected from 7.3, 10.5, 12.8, 14.5, 15.2, 16.4, 20.3, 21.3, 21.6, and 27.0.
9. The solid form of embodiment 1, wherein the form has at least ten XRPD peaks, in terms of 2-theta (±0.2 degrees), selected from 7.3, 10.5, 12.8, 14.5, 15.2, 16.4, 20.3, 21.3, 21.6, and 27.0.
10. The solid form of any one of embodiments 1-9, wherein the form has an XRPD pattern as substantially shown in FIG. 1.
11. The solid form of any one of embodiments 1-9, having an endothermic peak with an onset temperature (±3° C.) at 191.7° C. and a maximum at 193.6° C.
12. The solid form of any one of embodiments 1-9, wherein the form has a DSC thermogram substantially as shown in FIG. 2.
13. The solid form of any one of embodiments 1-12, wherein the form has a TGA thermogram substantially as shown in FIG. 3.
14. A salt of a compound of Formula (I):

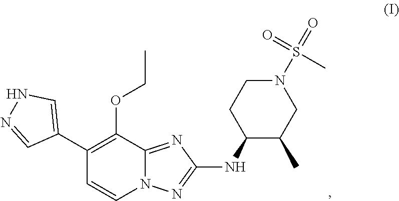 [SMILES: CCOc1c(-c2cn[nH]c2)ccn2nc(N[C@H]3CCN(S(C)(=O)=O)C[C@H]3C)nc12]

which is selected from:
a mono-maleate salt of the compound of Formula (I);
a di-besylate salt of the compound of Formula (I);
a mono-mesylate salt of the compound of Formula (I);
a di-tosylate salt of the compound of Formula (I);
a mono-hydrochloride salt of the compound of Formula (I); and a di-hydrochloride salt of the compound of Formula (I).
   15. The salt of embodiment 14, which is a mono-maleate salt of the compound of Formula (I).
   16. The salt of embodiment 15, which is crystalline.
   17. The salt of embodiment 15 or 16, wherein the salt has at least one XRPD peak, in terms of 2-theta (0.2 degrees), selected from 10.4, 11.6, 12.0, 14.1, 15.1, 17.2, 18.1, 19.1, 21.3, 21.9, 22.9, 24.2, and 25.9.
   18. The salt of embodiment 15 or 16, wherein the salt has at least two XRPD peaks, in terms of 2-theta (0.2 degrees), selected from 10.4, 11.6, 12.0, 14.1, 15.1, 17.2, 18.1, 19.1, 21.3, 21.9, 22.9, 24.2, and 25.9.
   19. The salt of embodiment 15 or 16, wherein the salt has at least three XRPD peaks, in terms of 2-theta (0.2 degrees), selected from 10.4, 11.6, 12.0, 14.1, 15.1, 17.2, 18.1, 19.1, 21.3, 21.9, 22.9, 24.2, and 25.9.
   20. The salt of embodiment 15 or 16, wherein the salt has at least four XRPD peaks, in terms of 2-theta (0.2 degrees), selected from 10.4, 11.6, 12.0, 14.1, 15.1, 17.2, 18.1, 19.1, 21.3, 21.9, 22.9, 24.2, and 25.9.
   21. The salt of embodiment 15 or 16, wherein the salt has at least five XRPD peaks, in terms of 2-theta (0.2 degrees), selected from 10.4, 11.6, 12.0, 14.1, 15.1, 17.2, 18.1, 19.1, 21.3, 21.9, 22.9, 24.2, and 25.9.
   22. The salt of embodiment 15 or 16, wherein the salt has at least ten XRPD peaks, in terms of 2-theta (0.2 degrees), selected from 10.4, 11.6, 12.0, 14.1, 15.1, 17.2, 18.1, 19.1, 21.3, 21.9, 22.9, 24.2, and 25.9.
   23. The salt of embodiment 15 or 16, wherein the salt has an XRPD pattern as substantially shown in FIG. 4.
   24. The salt of any one of embodiments 15-23, having an endothermic peak with an onset temperature (±3° C.) at 180.4° C. and a maximum temperature (±3° C.) at 181.8° C.
   25. The salt of any one of embodiments 15-23, wherein the salt has a DSC thermogram substantially as shown in FIG. 5.
   26. The salt of any one of embodiments 15-25, wherein the salt has a TGA thermogram substantially as shown in FIG. 6.
   27. The salt of embodiment 14, which is a di-besylate salt of the compound of Formula (I).
   28. The salt of embodiment 27, which is crystalline.
   29. The salt of embodiment 27 or 28, wherein the salt has at least one XRPD peak, in terms of 2-theta (0.2 degrees), selected from 6.3, 9.9, 12.1, 12.6, 15.9, 17.4, 18.7, 19.0, 19.6, and 25.1.
   30. The salt of embodiment 27 or 28, wherein the salt has at least two XRPD peaks, in terms of 2-theta (±0.2 degrees), selected from 6.3, 9.9, 12.1, 12.6, 15.9, 17.4, 18.7, 19.0, 19.6, and 25.1.
   31. The salt of embodiment 27 or 28, wherein the salt has at least three XRPD peaks, in terms of 2-theta (±0.2 degrees), selected from 6.3, 9.9, 12.1, 12.6, 15.9, 17.4, 18.7, 19.0, 19.6, and 25.1.
   32. The salt of embodiment 27 or 28, wherein the salt has at least four XRPD peaks, in terms of 2-theta (±0.2 degrees), selected from 6.3, 9.9, 12.1, 12.6, 15.9, 17.4, 18.7, 19.0, 19.6, and 25.1.
   33. The salt of embodiment 27 or 28, wherein the salt has at least five XRPD peaks, in terms of 2-theta (±0.2 degrees), selected from 6.3, 9.9, 12.1, 12.6, 15.9, 17.4, 18.7, 19.0, 19.6, and 25.1.
   34. The salt of embodiment 27 or 28, wherein the salt has at least ten XRPD peaks, in terms of 2-theta (0.2 degrees), selected from 6.3, 9.9, 12.1, 12.6, 15.9, 17.4, 18.7, 19.0, 19.6, and 25.1.
   35. The salt of embodiment 27 or 28, wherein the salt has an XRPD pattern as substantially shown in FIG. 7.
   36. The salt of any one of embodiments 27-35, having an endothermic peak with an onset temperature (±3° C.) at 160.4° C. and a maximum temperature (±3° C.) at 163.4° C.
   37. The salt of any one of embodiments 27-35, wherein the salt has a DSC thermogram substantially as shown in FIG. 8.
   38. The salt of any one of embodiments 27-37, wherein the salt has a TGA thermogram substantially as shown in FIG. 9.
   39. The salt of embodiment 14, which is a mono-mesylate salt of the compound of Formula (I).
   40. The salt of embodiment 39, which is crystalline.
   41. The salt of embodiment 39 or 40, wherein the salt has at least one XRPD peak, in terms of 2-theta (0.2 degrees), selected from 4.8, 7.0, 11.9, 14.1, 14.9, 17.7, 18.9, 20.2, 22.1, and 26.1.
   42. The salt of embodiment 39 or 40, wherein the salt has at least two XRPD peaks, in terms of 2-theta (0.2 degrees), selected from 4.8, 7.0, 11.9, 14.1, 14.9, 17.7, 18.9, 20.2, 22.1, and 26.1.
   43. The salt of embodiment 39 or 40, wherein the salt has at least three XRPD peaks, in terms of 2-theta (0.2 degrees), selected from 4.8, 7.0, 11.9, 14.1, 14.9, 17.7, 18.9, 20.2, 22.1, and 26.1.
   44. The salt of embodiment 39 or 40, wherein the salt has at least four XRPD peaks, in terms of 2-theta (0.2 degrees), selected from 4.8, 7.0, 11.9, 14.1, 14.9, 17.7, 18.9, 20.2, 22.1, and 26.1.
   45. The salt of embodiment 39 or 40, wherein the salt has at least five XRPD peaks, in terms of 2-theta (0.2 degrees), selected from 4.8, 7.0, 11.9, 14.1, 14.9, 17.7, 18.9, 20.2, 22.1, and 26.1.
   46. The salt of embodiment 39 or 40, wherein the salt has at least ten XRPD peaks, in terms of 2-theta (0.2 degrees), selected from 4.8, 7.0, 11.9, 14.1, 14.9, 17.7, 18.9, 20.2, 22.1, and 26.1.
   47. The salt of embodiment 39 or 40, wherein the salt has an XRPD pattern as substantially shown in FIG. 10.
   48. The salt of any one of embodiments 39-47, having a first endothermic peak with a maximum temperature (±3° C.) at 61.1° C. and a second endothermic peak with an onset temperature (±3° C.) at 134.4° C. and a maximum temperature (±3° C.) at 150.1° C.
   49. The salt of any one of embodiments 39-47, wherein the salt has a DSC thermogram substantially as shown in FIG. 11.
   50. The salt of any one of embodiments 39-49, wherein the salt has a TGA thermogram substantially as shown in FIG. 12.
   51. The salt of embodiment 14, which is a di-tosylate salt of the compound of Formula (I).
   52. The salt of embodiment 51, which is crystalline.
   53. The salt of embodiment 51 or 52, wherein the salt has at least one XRPD peak, in terms of 2-theta (0.2 degrees), selected from 5.7, 7.8, 8.1, 9.3, 13.7, 13.9, 16.2, 18.8, and 20.6.
   54. The salt of embodiment 51 or 52, wherein the salt has at least two XRPD peaks, in terms of 2-theta (0.2 degrees), selected from 5.7, 7.8, 8.1, 9.3, 13.7, 13.9, 16.2, 18.8, and 20.6.
   55. The salt of embodiment 51 or 52, wherein the salt has at least three XRPD peaks, in terms of 2-theta (±0.2 degrees), selected from 65.7, 7.8, 8.1, 9.3, 13.7, 13.9, 16.2, 18.8, and 20.6.
   56. The salt of embodiment 51 or 52, wherein the salt has at least four XRPD peaks, in terms of 2-theta (0.2 degrees), selected from 5.7, 7.8, 8.1, 9.3, 13.7, 13.9, 16.2, 18.8, and 20.6.
   57. The salt of embodiment 51 or 52, wherein the salt has at least five XRPD peaks, in terms of 2-theta (0.2 degrees), selected from 5.7, 7.8, 8.1, 9.3, 13.7, 13.9, 16.2, 18.8, and 20.6.
   58. The salt of embodiment 51 or 52, wherein the salt has at least eight XRPD peaks, in terms of 2-theta (0.2 degrees), selected from 5.7, 7.8, 8.1, 9.3, 13.7, 13.9, 16.2, 18.8, and 20.6.
   59. The salt of embodiment 51 or 52, wherein the salt has an XRPD pattern as substantially shown in FIG. 13.
   60. The salt of any one of embodiments 51-59, having an exothermic peak with an onset temperature (±3° C.) at 99.6° C. and a maximum temperature (±3° C.) at 110.5° C., and an endothermic peak with an onset temperature (±3° C.) at 216.1° C. and a maximum temperature (±3° C.) at 218.7° C.
   61. The salt of any one of embodiments 51-59, wherein the salt has a DSC thermogram substantially as shown in FIG. 14.
   62. The salt of any one of embodiments 51-61, wherein the salt has a TGA thermogram substantially as shown in FIG. 15.
   63. The salt of embodiment 14, which is a mono-hydrochloride salt of the compound of Formula (I).
   64. The salt of embodiment 63, which is crystalline.
   65. The salt of embodiment 63 or 64, wherein the salt has at least one XRPD peak, in terms of 2-theta (0.2 degrees), selected from 5.7, 8.5, 11.3, 14.1, 15.0, 18.4, 19.3, 20.5, 21.8, 22.8, and 25.7.
   66. The salt of embodiment 63 or 64, wherein the salt has at least two XRPD peaks, in terms of 2-theta (0.2 degrees), selected from 5.7, 8.5, 11.3, 14.1, 15.0, 18.4, 19.3, 20.5, 21.8, 22.8, and 25.7.
   67. The salt of embodiment 63 or 64, wherein the salt has at least three XRPD peaks, in terms of 2-theta (±0.2 degrees), selected from 5.7, 8.5, 11.3, 14.1, 15.0, 18.4, 19.3, 20.5, 21.8, 22.8, and 25.7.
   68. The salt of embodiment 63 or 64, wherein the salt has at least four XRPD peaks, in terms of 2-theta (±0.2 degrees), selected from 5.7, 8.5, 11.3, 14.1, 15.0, 18.4, 19.3, 20.5, 21.8, 22.8, and 25.7.
   69. The salt of embodiment 63 or 64, wherein the salt has at least five XRPD peaks, in terms of 2-theta (±0.2 degrees), selected from 5.7, 8.5, 11.3, 14.1, 15.0, 18.4, 19.3, 20.5, 21.8, 22.8, and 25.7.
   70. The salt of embodiment 63 or 64, wherein the salt has at least ten XRPD peaks, in terms of 2-theta (0.2 degrees), selected from 5.7, 8.5, 11.3, 14.1, 15.0, 18.4, 19.3, 20.5, 21.8, 22.8, and 25.7.
   71. The salt of embodiment 63 or 64, wherein the salt has an XRPD pattern as substantially shown in FIG. 16.
   72. The salt of any one of embodiments 63-71, having an endothermic peak with an onset temperature (±3° C.) at 196.0° C. and a maximum temperature (±3° C.) at 212.2° C.
   73. The salt of any one of embodiments 63-71, wherein the salt has a DSC thermogram substantially as shown in FIG. 17.
   74. The salt of any one of embodiments 63-73, wherein the salt has a TGA thermogram substantially as shown in FIG. 18.
   75. The salt of embodiment 14, which is a di-hydrochloride salt of the compound of Formula (I).
   76. The salt of embodiment 75, which is crystalline.
   77. The salt of embodiment 75 or 76, wherein the salt has at least one XRPD peak, in terms of 2-theta (±0.2 degrees), selected from 9.9, 10.7, 12.3, 13.0, 14.0, 15.2, 19.9, 21.8, 22.3, and 24.8.
   78. The salt of embodiment 75 or 76, wherein the salt has at least two XRPD peaks, in terms of 2-theta (±0.2 degrees), selected from 9.9, 10.7, 12.3, 13.0, 14.0, 15.2, 19.9, 21.8, 22.3, and 24.8.
   79. The salt of embodiment 75 or 76, wherein the salt has at least three XRPD peaks, in terms of 2-theta (±0.2 degrees), selected from 9.9, 10.7, 12.3, 13.0, 14.0, 15.2, 19.9, 21.8, 22.3, and 24.8.
   80. The salt of embodiment 75 or 76, wherein the salt has at least four XRPD peaks, in terms of 2-theta (±0.2 degrees), selected from 9.9, 10.7, 12.3, 13.0, 14.0, 15.2, 19.9, 21.8, 22.3, and 24.8.
   81. The salt of embodiment 75 or 76, wherein the salt has at least five XRPD peaks, in terms of 2-theta (±0.2 degrees), selected from 9.9, 10.7, 12.3, 13.0, 14.0, 15.2, 19.9, 21.8, 22.3, and 24.8.
   82. The salt of embodiment 75 or 76, wherein the salt has at least ten XRPD peaks, in terms of 2-theta (±0.2 degrees), selected from 9.9, 10.7, 12.3, 13.0, 14.0, 15.2, 19.9, 21.8, 22.3, and 24.8.
   83. The salt of embodiment 75 or 76, wherein the salt has an XRPD pattern as substantially shown in FIG. 19.
   84. The salt of any one of embodiments 75-83, having an endothermic peak with an onset temperature (±3° C.) at 182.1° C. and a maximum temperature (±3° C.) at 206.4° C.
   85. The salt of any one of embodiments 75-83, wherein the salt has a DSC thermogram substantially as shown in FIG. 20.
   86. The salt of any one of embodiments 75-85, wherein the salt has a TGA thermogram substantially as shown in FIG. 21.
   87. A pharmaceutical composition comprising the solid form of any one of embodiments 1-13 or the salt of any one of embodiments 14-86, and a pharmaceutically acceptable carrier.
   88. A method of inhibiting CDK2, comprising contacting the CDK2 with the solid form of any one of embodiments 1-13 or the salt of any one of embodiments 14-86.
   89. A method of inhibiting CDK2 in a patient, comprising administering to the patient the solid form of any one of embodiments 1-13 or the salt of any one of embodiments 14-86.
   90. A method of treating a disease or disorder associated with CDK2 in a patient, comprising administering to the patient a therapeutically effective amount of the solid form of any one of embodiments 1-13 or the salt of any one of embodiments 14-86.
   91. The method of embodiment 90, wherein the disease or disorder is associated with an amplification of the cyclin E1 (CCNE1) gene and/or overexpression of CCNE1.
   92. A method of treating a human subject having a disease or disorder associated with cyclin-dependent kinase 2 (CDK2), comprising administering to the human subject the solid form of any one of embodiments 1-13 or the salt of any one of embodiments 14-86, wherein the human subject has been previously determined to:
(i)
(a) have a nucleotide sequence encoding a p16 protein comprising the amino acid sequence of SEQ ID NO: 1; and/or
(b) have a cyclin dependent kinase inhibitor 2A (CDKN2A) gene lacking one or more inactivating nucleic acid substitutions and/or deletions;
(ii)
(a) have an amplification of the cyclin E1 (CCNE1) gene; and/or
(b) have an expression level of CCNE1 in a biological sample obtained from the human subject that is higher than a control expression level of CCNE1.
   93. A method of treating a human subject having a disease or disorder associated with cyclin-dependent kinase 2 (CDK2), comprising:
(i) identifying, in a biological sample obtained from the human subject:
   (a) a nucleotide sequence encoding a p16 protein comprising the amino acid sequence of SEQ ID NO:1; and/or
   (b) a cyclin dependent kinase inhibitor 2A (CDKN2A) gene lacking one or more inactivating nucleic acid substitutions;
(ii) identifying, in a biological sample obtained from the human subject:
   (a) an amplification of the cyclin E1 (CCNE1) gene; and/or
   (b) an expression level of CCNE1 that is higher than a control expression level of CCNE1; and
(iii) administering the solid form of any one of embodiments 1-13 or the salt of any one of embodiments 14-86 to the human subject.
   94. The method of embodiment 93, comprising:
(i) identifying, in a biological sample obtained from the human subject:
   (a) a nucleotide sequence encoding a p16 protein comprising the amino acid sequence of SEQ ID NO:1; and/or
   (b) a CDKN2A gene lacking one or more inactivating nucleic acid substitutions and/or deletions;
(ii) identifying, in a biological sample obtained from the human subject:
   (a) an amplification of the CCNE1 gene; and
(iii) administering the compound or the salt to the human subject.
   95. A method of evaluating the response of a human subject having a disease or disorder associated with cyclin-dependent kinase 2 (CDK2) to the solid form of any one of embodiments 1-13 or the salt of any one of embodiments 14-86, comprising:
(a) administering the compound or the salt, to the human subject, wherein the human subject has been previously determined to have an amplification of the cyclin E1 (CCNE1) gene and/or an expression level of CCNE1 that is higher than a control expression level of CCNE1;
(b) measuring, in a biological sample of obtained from the subject subsequent to the administering of step (a), the level of retinoblastoma (Rb) protein phosphorylation at the serine corresponding to amino acid position 780 of SEQ ID NO:3,
wherein a reduced level of Rb phosphorylation at the serine corresponding to amino acid position 780 of SEQ ID NO:3, as compared to a control level of Rb phosphorylation at the serine corresponding to amino acid position 780 of SEQ ID NO:3, is indicative that the human subject responds to the compound or the salt.
   96. The method of embodiment 95, wherein the disease or disorder is cancer.
   97. A process of preparing a solid form of any one of embodiments 1-13, comprising cooling a solution of the compound of Formula (I) in a solvent component comprising ethanol and water.
   98. The process of embodiment 97, wherein the solvent component comprises 6% water and 94% ethanol.
   99. The process of embodiment 97 or 98, wherein the solution is cooled to a temperature of 0° C.±3° C.
   100. The process of any one of embodiments 97-99, wherein the solution is prepared by heating a slurry of the compound of Formula (I) in the solvent component prior to said cooling.
   101. A process of preparing a compound of Formula (I), or a pharmaceutically acceptable salt thereof, a solid form of any one of embodiments 1-13; or the salt of any one of embodiments 14-86, comprising:
reacting a compound of Formula (1c):

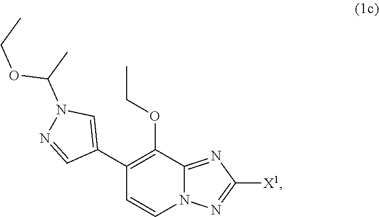

with a compound of Formula (1b):

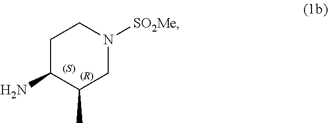

or a salt thereof, via a Buchwald coupling reaction, to form a compound of Formula (1a):

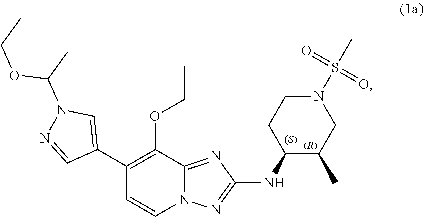

wherein X¹ is halo.
102. The process of embodiment 101, wherein X¹ is Br.
103. The process of embodiment 101 or 102, wherein the compound of formula (1b), or the salt thereof, is the HCl salt.
104. The process of any one of embodiments 101-103, wherein the Buchwald coupling reaction comprises reacting the compound of Formula (1c) with the compound of Formula (1b), or the salt thereof, in the presence of a Buchwald catalyst or precatalyst and a base.
105. The process of embodiment 104, wherein the Buchwald catalyst or precatalyst is a palladium catalyst.
106. The process of embodiment 104, wherein the palladium catalyst is [(2-di-tert-butylphosphino-3,6-dimethoxy-2′,4′,6′-triisopropyl-1,1′-biphenyl)-2-(2′-amino-1,1′-biphenyl)]palladium(II) methanesulfonate (t-BuBrett Phos Pd G3) or [tBuBrettPhos Pd(allyl)]OTf (Pd-175).
107. The process of any one of embodiments 104-106, wherein the base is an alkali metal alkoxide.
108. The process of any one of embodiments 104-106, wherein the base is sodium t-butoxide.
109. The process of any one of embodiments 101-108, further comprising deprotecting the compound of Formula (1a) to form the compound of Formula (I).
110. The process of embodiment 109, wherein the deprotecting is accomplished by reacting the compound of Formula (1a) with a strong acid.
111. The process of embodiment 110, wherein the strong acid is hydrochloric acid.
112. The process of any one of embodiments 101-111, wherein the compound of Formula (1c) is prepared by a process comprising:
reacting a compound of Formula (1d):

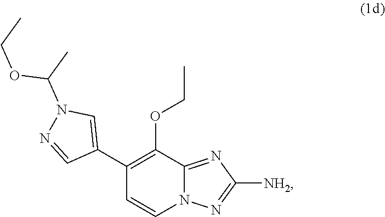 [SMILES: CCOc1c(-c2cnn(C(C)OCC)c2)ccn2nc(N)nc12]

or a salt thereof, with a halogenating agent to form the compound of Formula (1c)
113. The process of embodiment 112, wherein the halogenating agent is a brominating agent.
114. The process of embodiment 112, wherein the halogenating agent is Cu(X¹)₂.
115. The process of embodiment 112, wherein the halogenating agent is CuBr₂.
116. The process of any one of embodiments 112-115, wherein the compound of Formula (1d), or the salt thereof, is prepared by a process comprising:
reacting a compound of Formula 1(e):

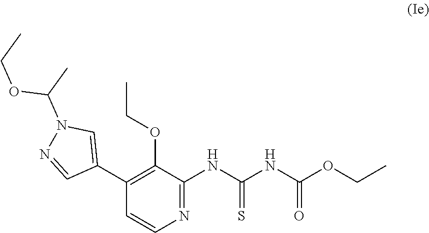 [SMILES: CCOC(=O)NC(=S)Nc1nccc(-c2cnn(C(C)OCC)c2)c1OCC]

with hydroxylamine HCl and a base component to form the compound of Formula (1d), or the salt thereof.
117. The process of embodiment 116, wherein the base component is a tertiary amine.
118. The process of embodiment 117, wherein the tertiary amine is ethyldiisopropylamine.
119. The process of any one of embodiments 116-118, wherein the compound of Formula (1e) is prepared by a process comprising:
reacting a compound of Formula (1f):

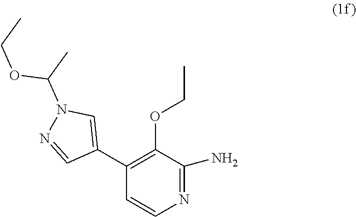 [SMILES: CCOc1c(-c2cnn(C(C)OCC)c2)ccnc1N]

with CH₃CH₂OC(O)—N═C═S to form the compound of Formula (1e).
120. The process of embodiment 119, wherein the compound of Formula (1f) is prepared by a process comprising:
reacting a compound of Formula (1h):

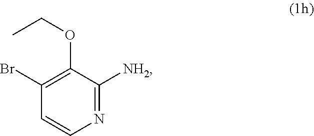

or a salt thereof, with a compound of Formula (1g):

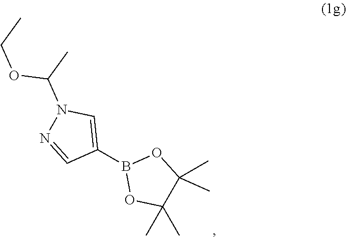 [SMILES: CCOC(C)n1cc(B2OC(C)(C)C(C)(C)O2)cn1]

via a Buchwald couple reaction to form the compound of Formula (1f).
121. The process of embodiment 120, wherein the compound of Formula (1h), or the salt thereof, is the HBr salt.
122. The process of embodiment 120 or 121, wherein the Buchwald coupling reaction comprises reacting the compound of Formula (1h), or the salt thereof, with the compound of Formula (1g) in the presence of a Buchwald catalyst or precatalyst and a base.
123. The process of embodiment 122, wherein the Buchwald catalyst or precatalyst, present for the reacting of the compound of Formula (1h), or the salt thereof, and the compound of Formula (1g), is a palladium catalyst.
124. The process of embodiment 122, wherein the Buchwald catalyst, present for the reacting of the compound of Formula (1h), or the salt thereof, and the compound of Formula (1g), is (2-dicyclohexylphosphino-2′,4′,6′-triisopropyl-1,1′-biphenyl)[2-(2′-amino-1,1′-biphenyl)]palladium(II) methanesulfonate (XPhos Pd G3).
125. The process of any one of embodiments 122-124, wherein the base, present for the reacting of the compound of Formula (1h), or the salt thereof, and the compound of Formula (1g), is an alkali metal phosphate.
126. The process of any one of embodiments 122-124, wherein the base, present for the reacting of the compound of Formula (1h), or the salt thereof, and the compound of Formula (1g), is sodium phosphate tribasic.
127. The process of any one of embodiments 120-126, wherein the compound of Formula (1h), or the salt thereof, is prepared by a process comprising:
reacting a compound of Formula (ii):

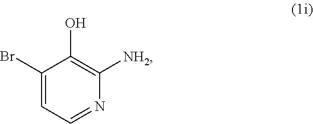

or a salt thereof, with an ethyl halide in the presence of a base to form the compound of Formula (1h), or the salt thereof.
128. The process of embodiment 127, wherein the ethyl halide is ethyl iodide.
129. The process of embodiment 127 or 128, wherein the base present for the reacting of the compound of Formula (1i), or the salt thereof, with the ethyl halide is a carbonate base.
130. The process of embodiment 129, wherein the carbonate base is cesium carbonate.
131. The process of any one of embodiments 127-130, wherein the compound of Formula (1i), or the salt thereof, is a HBr salt.
132. The process of any one of embodiments 101-111, wherein the compound of Formula (1c) is prepared by a process comprising:
reacting a compound of Formula (2a):

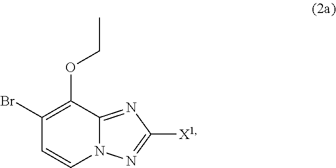

with a compound of Formula (2b):

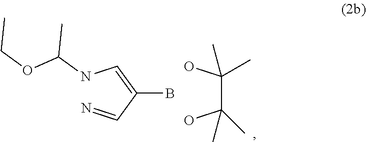

to form the compound of Formula (1c), in the presence of a Suzuki catalyst and a base, wherein X¹ is halo.
133. The process of embodiment 132, wherein X¹ is Br.
134. The process of embodiment 132 or 133, wherein the Suzuki catalyst is a palladium catalyst.
135. The process of embodiment 132 or 133, wherein the Suzuki catalyst is formed from a mixture of a phosphine ligand and a palladium (II) compound.
136. The process of embodiment 132 or 133, wherein the Suzuki catalyst is formed from a mixture of CataCXium A and palladium acetate.
137. The process of any one of embodiments 132-136, wherein the base, present for the reacting of the compound of Formula (2a) and the compound of Formula (2b), is an alkali metal phosphate.
138. The process of any one of embodiments 132-136, wherein the base, present for the reacting of the compound of Formula (2a) and the compound of Formula (2b), is sodium phosphate tribasic.
139. The process of any one of embodiments 132-138, wherein the compound of Formula (1b), or the salt thereof, is prepared by a process comprising:
reducing a compound of Formula (3a):

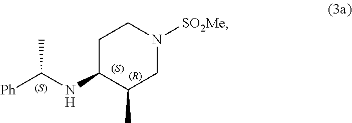

to form the compound of Formula (1b), or the salt thereof.
140. The process of embodiment 139, wherein the reducing is accomplished by reacting the compound of Formula (3a) with hydrogen gas in the presence of a palladium catalyst.
141. The process of any one of embodiments 139-140, wherein the compound of Formula (3a) is prepared by a process comprising:
reacting a compound of Formula (3c):

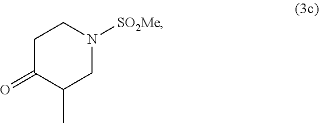

with a compound of Formula (3b):

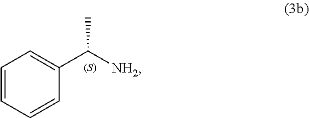

followed by crystallization to give the compound of Formula (3a).
142. The process of embodiment 141, wherein the compound of Formula (3c) is prepared by a process comprising:
reacting a compound of Formula (3d):

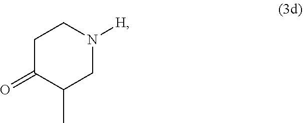

or a salt thereof, with methanesulfonyl chloride to form a compound of Formula (3c).
143. A compound selected from:

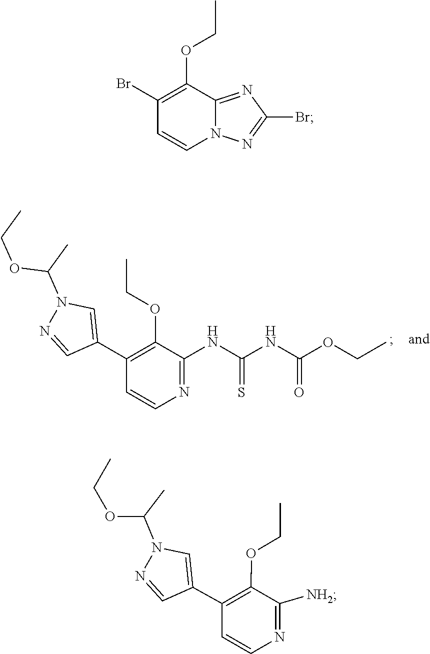 [SMILES: CCOc1c(Br)ccn2nc(Br)nc12 ; CCOC(=O)NC(=S)Nc1nccc(-c2cnn(C(C)OCC)c2)c1OCC ; CCOc1c(-c2cnn(C(C)OCC)c2)ccnc1N]

or a salt thereof.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

Experimental procedures for compounds, solid forms, and salts of the disclosure are provided below. Preparatory LC-MS purifications of some of the compounds prepared were performed on Waters mass directed fractionation systems. The basic equipment setup, protocols, and control software for the operation of these systems have been described in detail in the literature. See e.g., “Two-Pump at-Column Dilution Configuration for Preparative LC-MS,” K. Blom, J. Combi. Chem., 4, 295 (2002); “Optimizing Preparative LC-MS Configurations and Methods for Parallel Synthesis Purification,” K. Blom, R. Sparks, J. Doughty, G. Everlof, T. Haque, A. Combs, J. Combi. Chem., 5, 670 (2003); and “Preparative LC-MS Purification: Improved Compound Specific Method Optimization,” K. Blom, B. Glass, R. Sparks, A. Combs, J. Combi. Chem., 6, 874-883 (2004). The separated compounds were typically subjected to analytical liquid chromatography mass spectrometry (LCMS) for purity check under the following conditions: Instrument: Agilent 1100 series, LC/MSD; Column: Waters Sunfire™ C₁₈ 5 μm particle size, 2.1×5.0 mm; Buffers: mobile phase A: 0.025% TFA in water and mobile phase B: acetonitrile; gradient 2% to 80% of B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:

pH=2 purifications: Waters Sunfire™ C₁₈ 5 μm particle size, 19×100 mm column, eluting with mobile phase A: 0.1% TFA (trifluoroacetic acid) in water and mobile phase B: acetonitrile; the flow rate was 30 mL/minute, the separating gradient was optimized for each compound using the Compound Specific Method Optimization protocol as described in the literature (see “Preparative LCMS Purification: Improved Compound Specific Method Optimization,” K. Blom, B. Glass, R. Sparks, A. Combs, J. Comb. Chem., 6, 874-883 (2004)). Typically, the flow rate used with the 30×100 mm column was 60 mL/minute.

pH=10 purifications: Waters XBridge C₁₈ 5 μm particle size, 19×100 mm column, eluting with mobile phase A: 0.15% NH₄OH in water and mobile phase B: acetonitrile; the flow rate was 30 mL/minute, the separating gradient was optimized for each compound using the Compound Specific Method Optimization protocol as described in the literature (See “Preparative LCMS Purification: Improved Compound Specific Method Optimization,” K. Blom, B. Glass, R. Sparks, A. Combs, J. Comb. Chem., 6, 874-883 (2004)). Typically, the flow rate used with 30×100 mm column was 60 mL/minute.

### Example 1

### Synthesis of 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I

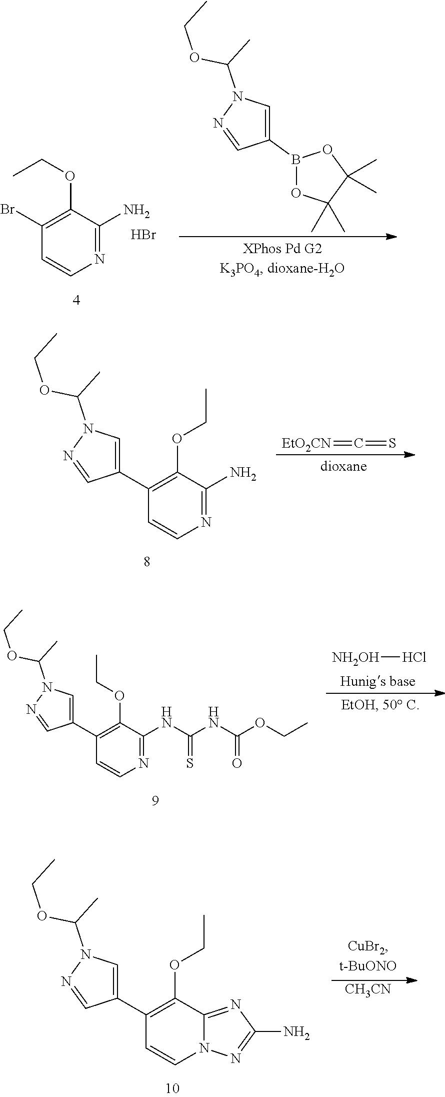

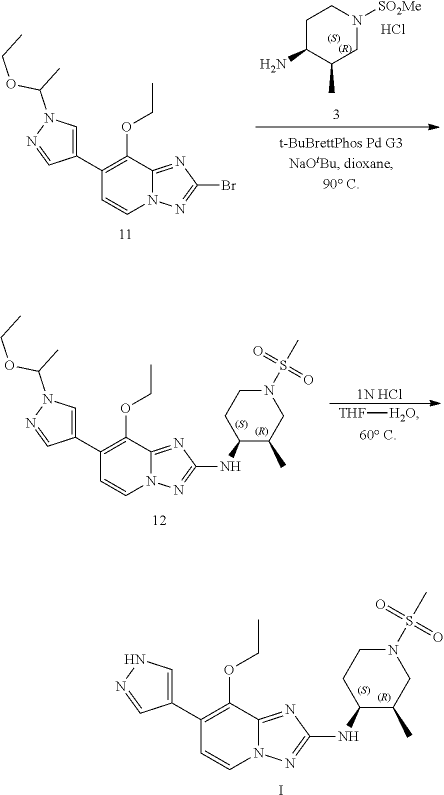

### Intermediate (8). 3-ethoxy-4-(1-(1-ethoxyethyl)-1H-pyrazol-4-yl)pyridin-2-amine

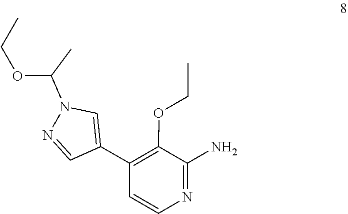 [SMILES: CCOc1c(-c2cnn(C(C)OCC)c2)ccnc1N]

4-Bromo-3-ethoxypyridin-2-amine (4) (prepared according to Example 4) (1.998 kg, 9.205 mol, 1 eq) and 1-(1-ethoxyethyl)-4-(4,4,5,5-tetramethyl-1,3,2-dioxaborolan-2-yl)-1H-pyrazole (2.94 kg, 11.0 mol, 1.2 eq) were charged to a 50 L reactor containing a solution of potassium phosphate tribasic (5.9 kg, 27.6 mmol, 3 eq) in water (10 L, 5 v). The slurry was diluted with dioxane (20 L, 10 v) and degassed via nitrogen bubbling for 50 min. The catalyst XPhos Pd G2 (21.7 g, 27.6 mmol, 0.003 eq) was charged and degassing continued for 15 min followed by the warming at 85° C. for 2.5 hrs until complete by HPLC analysis. The reaction mixture was cooled to rt and the organic layer was separated and washed with brine (6 L, 3 v). The aqueous layers were combined and extracted with ethyl acetate (10 L, 5 v). The organic layers were combined, filtered through a plug of Celite and concentrated to ˜6 L (3 v). The concentrated mixture was diluted with MTBE (18 L, 9 v) and warmed at 50° C. to form a clear solution. Heptane (18 L, 9 v) was charged slowly over −10 min and the solution was cooled to 1° C. over 2 hrs. The slurry was filtered, washed with heptane (4 L, 2 v), and dried on the filter to afford 8 (2.033 kg, 80% yield, >99% purity) as a yellow solid. ¹H NMR (400 MHz, DMSO-d₆) δ 8.38 (s, 1H), 8.00 (s, 1H), 7.65 (d, J=8.0 Hz, 1H), 6.79 (d, J=8.0 Hz, 1H), 5.77 (s, 2H), 5.62 (dd, J=8.0, 4.0 Hz, 1H), 3.72 (q, J=8.0 Hz, 2H), 3.46 (m, 1H), 3.23 (m, 1H), 1.63 (d, J=8.0 Hz, 3H), 1.33 (t, J=8.0 Hz, 3H), 1.05 (t, J=8.0 Hz, 3H); C₁₄H₂₀N₄O₂, (MW 276.34), LCMS (EI) m/e 277.13 (M⁺+H).

### Intermediate (9

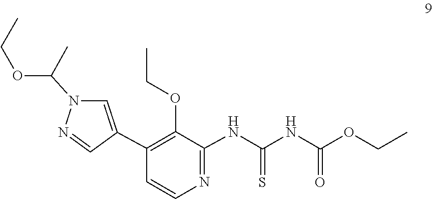 [SMILES: CCOC(=O)NC(=S)Nc1nccc(-c2cnn(C(C)OCC)c2)c1OCC]

A slurry of 8 (1.0 kg, 3.62 mol, 1 eq) in dioxane (5 L, 5 v) was cooled to 10° C. and O-ethyl carbonisothiocyanate (512 mL, 4.34 mol, 1.2 eq) was charged in one portion. The addition caused a mild exotherm from 9.1° C. to 21.7° C. and the reaction was agitated at rt for 16 hrs at which it was deemed complete by HPLC. The reaction was quenched by the addition of brine (2.5 L, 2.5 v) and water (1 L, 1 v) and the layers were separated. The aqueous layer was extracted with ethyl acetate (2.5 L, 2.5 v) and the organic layers were combined and concentrated to dryness. The crude 9 (assumed 1.475 kg, quant) was used directly in the next reaction as a thick orange oil. ¹H NMR (400 MHz, DMSO) δ 11.56 (s, 1H), 11.43 (s, 1H), 8.52 (s, 1H), 8.17 (d, J=5.1 Hz, 1H), 8.13 (s, 1H), 7.63 (d, J=5.1 Hz, 1H), 5.65 (q, J=5.9 Hz, 1H), 4.24 (q, J=7.1 Hz, 2H), 3.89 (q, J=7.0 Hz, 2H), 3.48 (dq, J=9.6, 7.0 Hz, 1H), 3.25 (dq, J=9.6, 7.0 Hz, 1H), 1.64 (d, J=6.0 Hz, 3H), 1.29 (m, 6H), 1.06 (t, J=7.0 Hz, 3H). C₁₈H₂₅N₅O₄S, (calc M+H: 408.1700), LCMS (EI) m/e 408.3 (M⁺+H).

### Intermediate (10). 8-ethoxy-7-(1-(1-ethoxyethyl)-1H-pyrazol-4-yl)-[1,2,4]triazolo[1,5-a]pyridin-2-amine

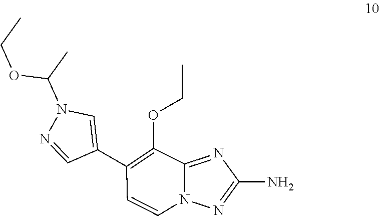 [SMILES: CCOc1c(-c2cnn(C(C)OCC)c2)ccn2nc(N)nc12]

Hünig's base (790 mL, 4.53 mol, 1.25 eq) was charged to a slurry of hydroxyamine hydrochloride (377 g, 5.43 mol, 1.5 eq) in ethanol (3 L, 2 v). A bleach scrubber was installed to capture hydrogen sulfide off-gassing. 9 (1.475 kg, 3.62 mol, 1 eq) in ethanol (4 L, 2.7 v) was charged slowly over 1 hr, and lines were rinsed with ethanol (400 mL, 0.3 v). Off-gassing began immediately with addition, with a mild exotherm (final temp 28° C.). The reaction mixture was warmed to 50° C., and a homogeneous solution was obtained. Following 3 hrs at 50° C., the reaction was deemed complete by HPLC and a thick solid product precipitated. Water (2.2 L, 1.5 v) was charged to dissolve the product and precipitate any sulfur-containing byproduct. The reaction mixture was filtered and solids were washed with ethanol/water (2:1, 700 mL, 0.5 v). The filtrate was concentrated to remove ethanol (˜7 L), leaving a slurry of 10 in water. The solids were dissolved in dichloromethane (7.3 L, 5 v) and 30% ammonium hydroxide (2.9 L, 2 v) was charged. The layers were separated, and the aqueous layer was extracted with dichloromethane (700 mL, 0.5 v). The organics were washed twice with 15% ammonium hydroxide (1.5 L, 1 v), back-extracting the aqueous layer with dichloromethane (700 mL, 0.5 v). The combined organic layers were washed with brine (3 L, 2 v) and the aqueous layer was extracted with dichloromethane (700 mL, 0.5 v). The combined organics were concentrated to ˜4-5 v (removing ˜7 L) and isopropyl acetate (6.3 L, 4.3 v) was charged. The remaining dichloromethane (˜3 L) was removed in vacuo and the slurry was transferred to a 22 L round bottom flask with isopropyl acetate (1 L, 0.7 v). The slurry was diluted with heptane (3.7 L, 2.5 v) and agitated at 80° C. for 1 hr followed by cooling to rt slowly overnight. The slurry was filtered, washed with isopropyl acetate/heptane (2:1, 700 mL, 0.5 v) and dried in a vacuum oven at 40° C. to afford 10 (920 g, 80% yield) as a white solid. ¹H NMR (400 MHz, DMSO) δ 8.47 (s, 1H), 8.27 (d, J=7.0 Hz, 1H), 8.12 (s, 1H), 7.17 (d, J=7.0 Hz, 1H), 5.99 (s, 2H), 5.62 (q, J=5.9 Hz, 1H), 4.56 (q, J=7.1 Hz, 2H), 3.47 (dq, J=9.6, 7.0 Hz, 1H), 3.26 (dq, J=9.6, 7.1 Hz, 1H), 1.64 (d, J=5.9 Hz, 3H), 1.35 (t, J=7.0 Hz, 3H), 1.06 (t, J=7.0 Hz, 3H). C₁₅H₂₀N₆O₂, (calc M+H: 317.1721), LCMS (EI) m/e 317.1 (M⁺+H).

### Intermediate (11). 2-bromo-8-ethoxy-7-(1-(1-ethoxyethyl)-1H-pyrazol-4-yl)-[1,2,4]triazolo[1,5-a]pyridine

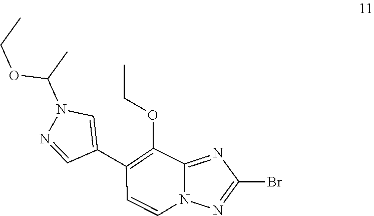 [SMILES: CCOc1c(-c2cnn(C(C)OCC)c2)ccn2nc(Br)nc12]

Copper (II) bromide (449 g, 2.0 mol, 1.05 eq) was charged to an ambient slurry of (605 g, 1.9 mol, 1 eq) in acetonitrile (9 L, 15 v), resulting in a dark solution. The reaction mixture was cooled to 5° C. and tert-butyl nitrite (607 mL, 4.6 mol, 2.4 eq) was added in one portion. The reaction mixture was allowed to slowly warm to rt overnight (allowing the ice bath to melt for slower warming). Following 16 hrs, the reaction was complete by HPLC and was quenched by the addition of 15% ammonium hydroxide (5 L, 8 v). Acetonitrile (˜6-8 L) was removed in vacuo and the mixture was diluted with dichloromethane (3 L, 5 v). The layers were separated and the aqueous portion was extracted with dichloromethane (0.5 L, 1 v). The organics were washed with 15% ammonium hydroxide (1.8 L, 3 v), and the aqueous layer was extracted with dichloromethane (0.5 L, 1 v). The hazy organic layer was washed twice with brine (1.2 L, 2 v), extracting each aqueous layer with dichloromethane (0.5 L, 1 v). The organic layer was slurried for 1 hr at rt with SiliaMetS Thiol (150 g, 0.25×wt), then filtered through a small bed of Celite and washed with dichloromethane (3×0.5 L, 1 v). The filtrate was concentrated to 1.2-1.5 L (˜2-2.5 v) in vacuo, and the resultant slurry was warmed at reflux and diluted with heptane (3 L, 5 v). The slurry was warmed at 40° C. for 1 hr, then cooled to rt and aged for 30 min. The slurry was filtered, washed with heptane/DCM (2:1, 1.4 L, 2 v). The solids were dried at 40° C. in a vacuum oven to afford 11 (536 g, 74%) as a white solid. ¹H NMR (400 MHz, DMSO) δ 8.68 (d, J=7.1 Hz, 1H), 8.58 (s, 1H), 8.21 (s, 1H), 7.58 (d, J=7.1 Hz, 1H), 5.65 (q, J=5.9 Hz, 1H), 4.65 (q, J=7.0 Hz, 2H), 3.48 (dq, J=9.6, 7.0 Hz, 1H), 3.27 (dq, J=9.6, 7.0 Hz, 1H), 1.65 (d, J=6.0 Hz, 3H), 1.40 (t, J=7.0 Hz, 3H), 1.07 (t, J=7.0 Hz, 3H). C₁₅H₁₈BrN₅O₂, (calc M+H: 380.0717), LCMS (EI) m/e 380.0717 (M⁺+H).

### Intermediate (12). 8-ethoxy-7-(1-(1-ethoxyethyl)-1H-pyrazol-4-yl)-N-((3R,4S)-3-methyl-1-(methylsulfonyl)piperidin-4-yl)-[1,2,4]triazolo[1,5-a]pyridin-2-amine

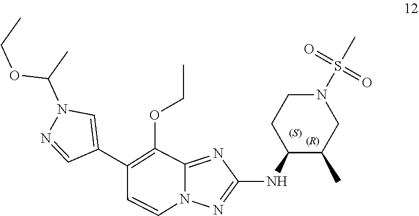

A solution of 11 (1.5 kg, 3.9 mol, 1 eq), 3 (1.08 kg, 4.7 mol, 1.2 eq) (prepared according to Example 3), sodium tert-butoxide (1.9 kg, 19.7 mol, 5 eq) and t-BuBrettPhos Pd G3 (101 g, 118 mmol, 0.03 eq) in dioxane was degassed with nitrogen bubbling for 1 hr. The slurry was heated to 90° C., forming a homogeneous solution, and stirred at that temperature for 2 hrs until complete by HPLC analysis. The reaction mixture was cooled to rt and quenched by the addition of water (7.5 L, 5 v). The layers were separated and the aqueous layer was extracted twice with ethyl acetate (15 L and 7.5 L, 10 v and 5 v). The combined organic layers were treated with a solution of N-acetyl cysteine (690 g) and potassium phosphate tribasic (990 g) in water (7.5 L) at 60° C. for 3 hrs. The wash to remove palladium was cooled to rt and the layers allowed to separate. The aqueous layer was extracted twice with ethyl acetate (7.5 L, 5 v) and the organics were concentrated to ˜20 L (13 v) in vacuo and slurried at rt with Carbon C-941 (300 g, 0.20 wt) and SiliMetS Thiol (300 g, 0.20 wt) for no longer than 8 hrs. The slurry was filtered through a pad of Celite, rinsing with ethyl acetate (7.5 L, 5 v). The filtrate was concentrated to afford a crude oil of 12 (assumed quant), which was used directly in the next step. ¹H NMR (600 MHz, DMSO-d₆) δ 8.48 (s, 1H), 8.33 (d, J=6.0 Hz, 1H), 8.13 (s, 1H), 7.18 (d, J=12.0 Hz, 1H), 6.76 (d, J=6.0 Hz, 1H), 5.62 (q, J=6.0 Hz, 1H), 4.55 (q, J=6.0 Hz, 2H), 3.88 (m, 1H), 3.47 (m, 1H), 3.26 (m, 2H), 3.12 (m, 3H), 2.87 (s, 3H), 2.18 (m, 1H), 1.85 (m, 1H), 1.76 (m, 1H), 1.64 (d, J=6.0 Hz, 3H), 1.36 (t, J=6.0 Hz, 3H), 1.06 (t, J=6.0 Hz, 3H), 0.93 (d, J=6.0 Hz, 3H); C₂₂H₃₃N₇O₄S, (MW 491.61), LCMS (EI) m/e 492.2 (M⁺+H).

### Compound of Formula (I). 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine

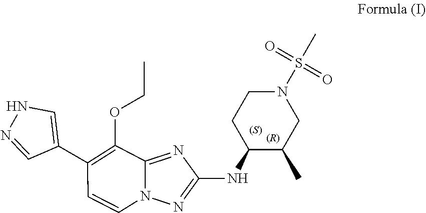

To a solution of 12 (1.9 kg, 3.9 mol, 1 eq) (or 12 hemi-succinate) in TH (14 L, 7 v) was added 1N hydrochloric acid (9 L, 9.0 mol, 2.3 eq) and the solution was warmed at 60° C. for 2 hrs until complete by HPLC. The reaction mixture was cooled to rt and neutralized with 16% sodium hydroxide (2 L, 9.3 mol). The reaction mixture was extracted twice with ethyl acetate (14 L and 8 L, 7 v and 4 v) and the combined organic layers were agitated with activated carbon C-941 (600 g, 0.32 wt) and SiliaMetS thiol (600 g, 0.32 wt) for 16 hrs. The slurry was filtered over a pad of Celite and washed with ethyl acetate (7.5 L, 5 v). The filtrate was concentrated to ˜7.5 L (5 v) and the slurry was diluted with heptane (4.5 L, 3 v), agitated for 30 min at rt, then cooled to 0° C. The solids were filtered and washed with ethyl acetate/heptane (5:3, 1.5 L, 1 v). The solids were dried on the filter to afford the crude compound of Formula (I) (1.312 kg, 80% yield).

Crude compound of Formula (I) (1.4 kg, 3.3 mol) was slurried in 6% water/ethanol (7 L, 5 v) and the slurry was warmed at reflux to obtain a clear solution. The mixture was cooled slowly to 0° C. and filtered, rinsing with ethanol (500 mL, 0.35 v). The solids were dried at 45° C. under vacuum for 3 days to afford the compound of Formula (I) (1.163 kg, 83% yield) as a white solid. ¹H NMR (500 MHz, DMSO-d6) δ 13.10 (s, 1H), 8.33 (s, 1H), 8.31 (d, J=10.0 Hz, 1H), 8.12 (s, 1H), 7.17 (d, J=10.0 Hz, 1H), 6.73 (d, J=10.0 Hz, 1H), 4.54 (dd, J=15.0, 10.0 Hz, 2H), 3.88 (m, 1H), 3.26 (m, 1H), 3.12 (m, 3H), 2.87 (s, 3H), 2.19 (m, 1H), 1.85 (m, 1H), 1.76 (m, 1H), 1.36 (t, J=10.0 Hz, 3H), 0.93 (d, J=5.0 Hz, 3H); C18H25N7O3S, (calc M+H: 420.1812), LCMS (EI) m/e 420.1818 (M++H).

### Example 2

### Synthesis of 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I

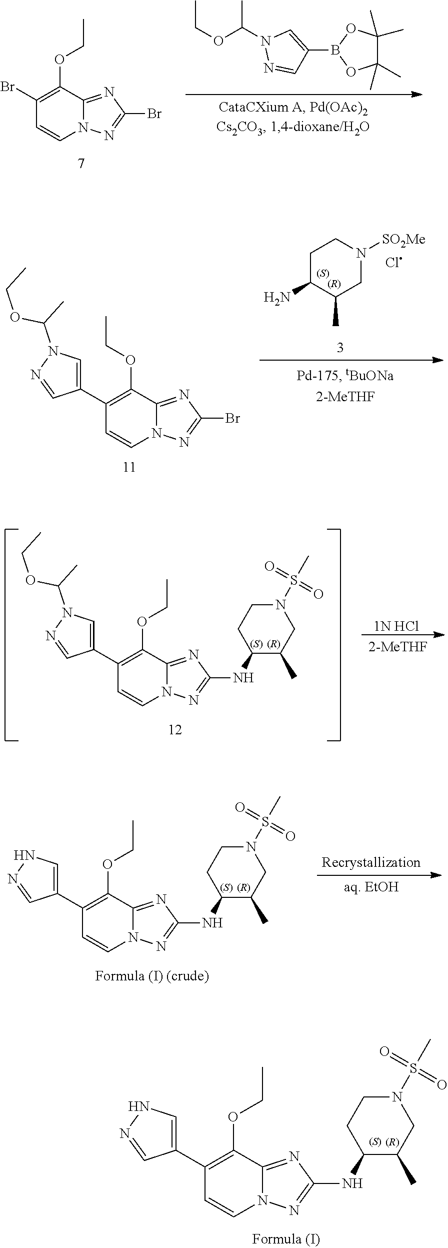

### Intermediate (11). 2-bromo-8-ethoxy-7-(1-(1-ethoxyethyl)-1H-pyrazol-4-yl)-[1,2,4]triazolo[1,5-a]pyridine (alternative synthesis

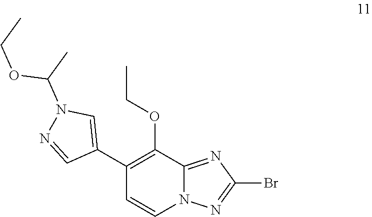 [SMILES: CCOc1c(-c2cnn(C(C)OCC)c2)ccn2nc(Br)nc12]

To degassed dioxane (0.84 L) was charged Pd(OAc)₂ (7.35 g, 0.5 mol %) and CataCXium A (25.8 g, 1.1 mol %) under nitrogen and the mixture was evacuated and refilled with nitrogen three times. The mixture was stirred at rt under nitrogen for no longer than 20 min. To a separate reactor was charged 7 (prepared according to Example 4) (2.10 kg), cesium carbonate (4.26 kg, 2 eq), 1-(1-ethoxyethyl)-4-(4,4,5,5-tetramethyl-1,3,2-dioxaborolan-2-yl)-1H-pyrazole (2.05 kg, 1.2 eq), dioxane (9.7 L, 5 vol in total including the dioxane utilized for in-situ catalyst formation) and water (5.3 L, 2.5 vol). The mixture was next purged with nitrogen for no longer than 30 min. After that period, the above-prepared catalyst was added to the mixture. The combined mixture was purged with nitrogen for 5 min. The mixture was then heated to 50° C. for no longer than 10 hrs. The reaction was monitored by HPLC until complete. Next was charged water (15.8 L, 7.5 v) slowly via an addition funnel. The resultant slurry was slowly cooled to rt and further to 0° C. and stirred for no longer than 30 min. The slurry was filtered and the solid was washed with water (3.2 L). The solid was dried at no greater than 50° C. to give 2.49 kg of 11.

Crude 11 (4.61 kg) was mixed with THF (18.4 L, 4 v based on weight of the isolated solid) and stirred at 50° C. to give a clear solution. Heptane (36.8 L, 8 v) was then charged at 50° C. The resultant slurry was cooled down to 15-30° C. slowly, and then further cooled to 0° C. The slurry was filtered and the solid was washed with heptane (4.6 L) and dried at no greater than 50° C. to obtain 4.91 kg (84%) of 11 in >99A % purity. ¹H NMR (400 MHz, DMSO-d₆) δ 8.67 (d, J=8.0 Hz, 1H), 8.58 (s, 1H), 8.21 (s, 1H), 7.58 (d, J=8.0 Hz, 1H), 5.65 (dd, J=12.0, 8.0 Hz, 1H), 4.65 (dd, J=12.0, 8.0 Hz, 2H), 3.48 (m, 1H), 3.27 (m, 1H), 1.65 (d, J=8.0 Hz, 3H), 1.40 (t, J=8.0 Hz, 3H), 1.07 (t, J=8.0 Hz, 3H); C₁₅H₁₈BrN₅O₂, (MW 380.25), LCMS (EI) m/e 380.0, 382.0 (M⁺+H).

### Intermediate (12). 8-ethoxy-7-(1-(1-ethoxyethyl)-1H-pyrazol-4-yl)-N-((3R,4S)-3-methyl-1-(methylsulfonyl)piperidin-4-yl)-[1,2,4]triazolo[1,5-a]pyridin-2-amine

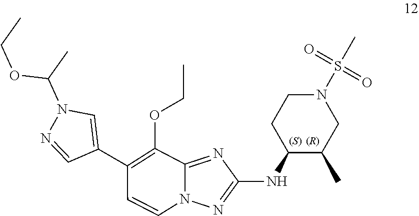

11 (4.1 kg, 1.0 eq), 3 (2.71 kg, 1.1 eq) (prepared according to Example 3), and NaOtBu (3.63 kg, 3.5 eq) were combined in 2-MeTHF (10 v). The solution was degassed and Pd-175 (8.41 g, 0.001 eq) was charged. The solution was further degassed and heated to 70° C. until complete by HPLC. The reaction was next cooled to rt and quenched by the addition of brine (4 v). The organics were then washed with brine (3 v). The crude 12 in 2-MeTHF was concentrated to ˜10 v and used directly in the next reaction (assumed quantitative).

### Alternative Synthesis of Intermediate 12 as the Hemi-Succinate

Alternatively, 11 (10 g, 1.0 eq), 3 (6.62 g, 1.1 eq), and NaOtBu (8.85 g, 3.5 eq) were combined in 2-MeTHF (10 v). The solution was degassed and Pd-175 (21 mg, 0.001 eq) was charged. The solution was further degassed and heated to 70° C. until complete by HPLC. The reaction was next cooled to room temperature and quenched by the addition of ammonium chloride (aq, 20 wt %, 6 v). The organics were then washed with brine (6 v). The crude 12 in 2-MeTHF was concentrated to ˜5 v and azeotropically dried by constant distillation with 2-MeTHF until KF<1.5%. Acetonitrile (5 v) was charged followed by succinic acid (2.3 g, 0.75 eq) portion-wise at 55° C. The resultant slurry was aged 1 h at 55° C. and cooled to rt overnight. Filtration followed by washing with acetonitrile/2-MeTHF (1:1, 2 v) afforded 12 hemi-succinate as a white solid (13.8 g, 95% yield). 1H NMR (400 MHz, DMSO) δ 12.18 (s, 1H), 8.48 (s, 1H), 8.33 (d, J=7.0 Hz, 1H), 8.13 (s, 1H), 7.18 (d, J=7.0 Hz, 1H), 6.75 (d, J=8.6 Hz, 1H), 5.62 (q, J=5.9 Hz, 1H), 4.55 (q, J=7.1 Hz, 2H), 3.88 (dp, J=10.8, 3.8 Hz, 1H), 3.47 (dq, J=9.6, 7.0 Hz, 1H), 3.25 (dt, J=9.8, 6.9 Hz, 2H), 3.13 (m, 3H), 2.86 (s, 3H), 2.42 (s, 2H), 2.18 (tt, J=7.7, 4.3 Hz, 1H), 1.90-1.71 (m, 1H), 1.64 (d, J=6.0 Hz, 3H), 1.36 (t, J=7.1 Hz, 3H), 1.06 (t, J=7.0 Hz, 3H), 0.93 (d, J=6.9 Hz, 3H). C22H33N7O4S, (calc M+H: 492.2387), LCMS (EI) m/e 492.2389 (M⁺+H).

### Compound of Formula (I). 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (crude

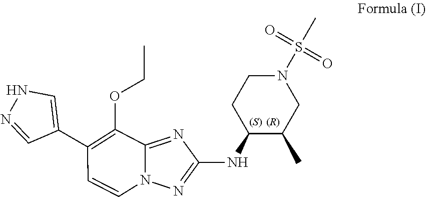

To a clean, inerted reactor was charged 12 (or 12 hemi-succinate) in 2-MeTHF, followed by 1N aqueous HCl (32.3 L, 3 eq). The mixture was heated to 70±5° C. while concentrating the reaction mixture to remove acetaldehyde until reaction was complete as indicated by HPLC. Upon completion, the reaction was cooled to 15-30° C. and the aqueous layer containing the product was removed. The organic layer was extracted twice with 1N HCl (2×5.3 L, 2 v total) and the combined aqueous layers were diluted with 2-MeTHF (45 L). The reaction mixture was neutralized by the addition of 5 N sodium hydroxide (8.4 L) to a pH of ˜8-9. The organic layer containing the product was separated and the aqueous layer extracted with 2-MeTHF (10.6 L, 2 v). The combined organic layers were treated at 40° C. with carbon C-941 (530 g, 20% w/w) and SiliaMetS imidazole (530 g, 20% w/w) for no longer than 12 hrs. The mixture was cooled to 15-30° C. and filtered through Celite. The collected solids were washed with 2-MeTHF (21.2 L). The filtrate containing the product was washed twice with water (7.95 L). The organic layer was concentrated while charging 2-Me THE at NMT 50° C. to remove residual water. The reaction mixture was then diluted with heptane (10 v), maintaining a temperature of >45° C. Following aging at 50° C. for −30 min, the reaction mixture was cooled to 15-30° C. and filtered. The product was washed with heptane (10.6 L, 2 v) and dried at no greater than 50° C. to give the crude compound of Formula (I).

### Solid Form of the Compound of Formula (I). 8-ethoxy-N-((3R,4S)-3-methyl-1-(methylsulfonyl)piperidin-4-yl)-7-(1H-pyrazol-4-yl)-[1,2,4]triazolo[1,5-a]pyridin-2-amine (Formula (I

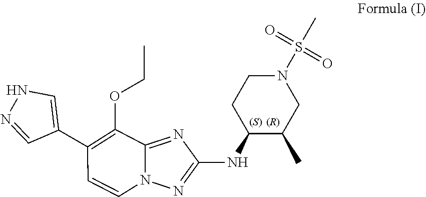

A 10% aqueous ethanol solution (42 L) was prepared in a clean, inerted reactor. Next, the crude compound of Formula (I) (4.2 kg) from the previous step was charged and the contents of the reactor were heated to 50-80° C. until a solution was observed. The solution was cooled to 47-57° C. and C-941 activated charcoal (420 g) and SiliaMetS imidazole (420 g) were added. The mixture was agitated for no longer than 12 hrs. The mixture was cooled to 35-50° C. and filtered through Celite. The filtrate was then polish filtered into a clean inert reactor. Polish filtered ethanol was charged and distilled to remove water from the solution until Karl Fisher analysis met the criterion specified in the batch record. The mixture was cooled to −5-5° C. The solids were washed with cold ethanol (4.2 L) and dried in a vacuum oven at no greater than 50° C.

### Example 3

### Synthesis of (3R,4S)-3-methyl-1-(methylsulfonyl)piperidin-4-amine hydrochloride (3

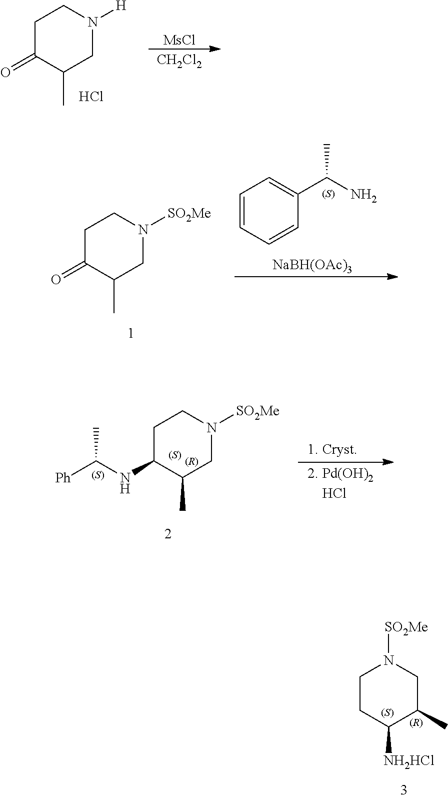

### Intermediate (1). 3-Methyl-1-(methylsulfonyl)piperidin-4-one

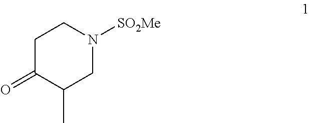

To a 3-liter 3-neck round bottom flask with mechanical stirrer and thermocouple under nitrogen was added 3-methylpiperidin-4-one hydrochloride (106.2 g, 710 mmol), DCM (708 mL) and methanesulfonyl chloride (60.4 mL, 781 mmol). The reaction mixture was stirred and turned into a solution within a few minutes. Triethylamine (218 mL, 1562 mmol) was slowly added via an addition funnel over 30 min. The stirring was continued at room temperature overnight (12 hrs). The suspended solids were filtered off as a waste and the solid was rinsed with DCM (300 mL) in three portions. The filtrate was then washed with aqueous 0.5 N HCl to remove the TEA residue. The DCM layer was washed further with brine solution (60 mL). The DCM layer was then concentrated to dryness as a pale yellow solid (132 g, 97% yield) on a rotary evaporator under reduced pressure. The resulting crude product, 3-methyl-1-(methylsulfonyl)piperidin-4-one (1, 132.1 g, 691 mmol, 97% yield), was used in the next reaction without further purification. ¹H NMR (400 MHz, DMSO) δ 3.85-3.74 (m, 2H), 3.21 (td, J=11.9, 3.8 Hz, 1H), 2.98 (s, 3H), 2.88 (t, J=11.5 Hz, 1H), 2.78-2.67 (m, 1H), 2.70-2.59 (m, 1H), 2.34 (dt, J=14.7, 3.6 Hz, 1H), 0.95 (d, J=6.6 Hz, 3H). C₇H₁₃NO₃S, (calc M+H: 192.0689), LCMS (EI) m/e 192.1 (M⁺+H).

### Intermediate (2). (3R,4S)-3-Methyl-1-(methylsulfonyl)-N—((S)-1-phenylethyl)piperidin-4-amine

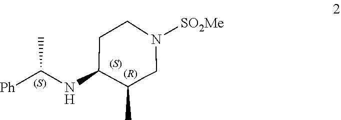

To a solution of 1 (132.4 g, 1 eq) in THE (1324 mL, 10 v) was charged (S)-1-phenylethan-1-amine (134 mL, 1.5 eq) and DIPEA (145 mL, 1.2 eq). Sodium triacetoxyborohydride (STAB; 220 g, 1.5 eq) was added portion-wise while maintaining 20-25° C. (delayed exotherm can be seen). The reaction mixture was agitated for 1 h until complete by HPLC, and then quenched by the slow addition of methanol (132 mL, 1 v). The mixture was concentrated to minimal volume and diluted with dichloromethane (1324 mL, 10 v). The organics were washed twice with saturated sodium bicarbonate (6 v and 4 v), then brine (2 v), and then concentrated to near dryness. The crude was diluted with EtOAc (4 v), and concentrated to a minimal volume to remove residual DCM. The crude solids were then slurried in EtOAc (4 v) and warmed at 70° C. to form a homogeneous mixture. The reaction mixture was cooled to 60° C. and seeded with 2, cooled to 50° C., and aged for crystal growth for 6 hrs, followed by slow cooling to 0° C. The solids were filtered, washed with EtOAc (1 v), and dried to afford 2 (115 g, 56.3% yield, 94:0.5:5:5 dr) as a white solid.

A diastereomeric mixture of 2 (115 g, 1 eq, 94:0.5:5:5 dr) was warmed at reflux in EtOAc (693 mL, 6 v) to afford a clear to almost clear solution. The solution was cooled at 60° C. and seeded as auto seeding was not seen. The mixture was aged at 50-60° C. for 5 hrs to afford a thick slurry and continued to cool to rt slowly. The solids were isolated via filtration, washed with EtOAc (1 v), and dried in a vacuum oven to afford 2 (92.4 g, 80% yield, 99.27:0:0.73:0 dr) as a white solid. The crystallization was repeated until desired dr was obtained (typically two recrystallizations). ¹H NMR (400 MHz, DMSO) δ 7.39-7.32 (m, 2H), 7.35-7.26 (m, 2H), 7.25-7.16 (m, 1H), 3.77 (q, J=6.6 Hz, 1H), 3.37-3.27 (m, 1H), 3.18 (ddd, J=11.7, 4.5, 1.8 Hz, 1H), 2.75 (s, 3H), 2.70-2.59 (m, 2H), 2.37 (dt, J=9.1, 4.5 Hz, 1H), 1.99 (q, J=4.8, 4.0 Hz, 2H), 1.45 (m, 2H), 1.23 (d, J=6.6 Hz, 3H), 0.94 (d, J=6.9 Hz, 3H). C₁₅H₂₄N₂O₂S, (calc M+H: 297.1631), LCMS (EI) m/e 297.2 (M⁺+H).

### Intermediate (3). (3R,4S)-3-Methyl-1-(methylsulfonyl)piperidin-4-amine hydrochloride

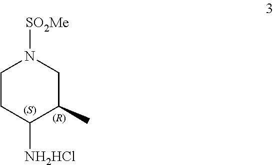

To a solution of 2 (10.31 g, 1 eq) in methanol (200 mL, 20 v) was charged Pd(OH)₂ (2.4 g, 20% wt) and HCl (3N in MeOH; 11.6 mL, 1 eq). The reaction mixture was degassed and subjected to H₂ (60 psi) for 24 hrs. Additional Pd(OH)₂ (0.73 g, 20% wt) was charged and the mixture was subject to H₂ for an additional 6 hrs at which point it was complete by HPLC analysis. The reaction mixture was filtered through Celite, washing with methanol (3×60 mL, 6 v) and concentrated to dryness in vacuo. The crude solids were triturated in heptane (40 mL, 4 v) at 60° C. for 6 hrs. The slurry was cooled to rt, filtered, and washed with heptane (20 mL, 2 v) to afford 3 (7.5 g, 94% yield) as the HCl salt. ¹H NMR (400 MHz, DMSO) δ 8.38-8.33 (s, 3H), 3.49-3.39 (m, 1H), 3.33-3.24 (m, 2H), 3.02 (dd, J=12.1, 3.3 Hz, 1H), 2.98-2.89 (m, 1H), 2.87 (s, 3H), 2.24-2.14 (m, 1H), 1.83 (m, 2H), 0.97 (d, J=7.0 Hz, 3H). C₇H₁₆N₂O₂S, (calc M+H: 193.1005), LCMS (EI) m/e 193.1 (M⁺+H).

### Example 4

### Synthesis of 2,7-dibromo-8-ethoxy-[1,2,4]triazolo[1,5-a]pyridine (7

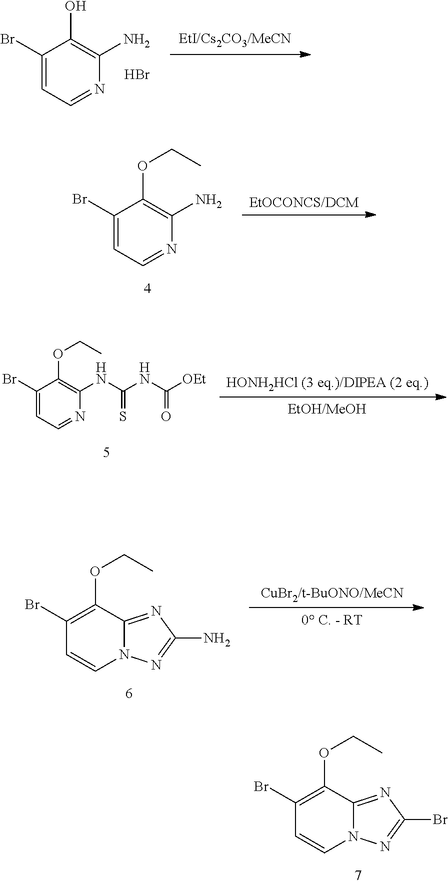 [SMILES: Nc1nccc(Br)c1O ; Br ; CCOc1c(Br)ccnc1N ; CCOC(=O)NC(=S)Nc1nccc(Br)c1OCC ; CCOc1c(Br)ccn2nc(N)nc12 ; CCOc1c(Br)ccn2nc(Br)nc12]

### Intermediate (4). 4-bromo-3-ethoxypyridin-2-amine

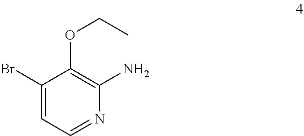

To a 5 L round bottomed flask equipped with mechanical stirrer and reflux condenser under nitrogen environment was combined 2-amino-4-bromopyridin-3-ol hydrobromide (245.0 g, 871 mmol), cesium carbonate (568 g, 1743 mmol) and acetonitrile (2000-2500 mL, 711 mmol). The mixture was stirred 15 minutes and iodoethane (105 mL, 1307 mmol) was added. The reaction mixture was heated at 65-70° C. for 18 hrs and was noted to be complete by HPLC analysis. The reaction mixture was cooled and allowed to stir at room temperature for 30 min. The resulting reaction slurry was filtered over Celite to remove salts and the cake washed with dichloromethane. The filtrate containing the product was concentrated to dryness and 500 mL methylene chloride was added. Additional cesium carbonate precipitated and was removed by filtration. The filtrate was concentrated and subjected to silica plug using a 750 g Biotage column using a gradient of ethyl acetate/dichloromethane from 0 to 25%. Clean fractions were combined and concentrated on a rotary evaporator.

The resulting solid was slurried in 1.25 to 1.5 volumes of heptane and heated to 98° C. The mixture was then cooled to 0° C. The solid was filtered and washed with ice cold heptane. The solid was dried by pulling air through the cake. 4-bromo-3-ethoxypyridin-2-amine (4) was obtained in 70-75% yield. ¹H NMR (400 MHz, DMSO) δ 7.53 (d, J=5.4 Hz, 1H), 6.73 (d, J=5.3 Hz, 1H), 6.13 (s, 2H), 3.91 (q, J=7.0 Hz, 2H), 1.35 (t, J=7.0 Hz, 3H). C₇H₉BrN₂O, (calc M+H: 216.9971), LCMS (EI) m/e 216.9 (M⁺+H).

### Intermediate (5

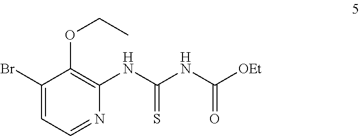 [SMILES: CCOC(=O)NC(=S)Nc1nccc(Br)c1OCC]

To a solution of 4 (44.7 g, 1 eq) in DCM (400 mL, 9 v) was added ethoxycarbonyl isothiocyanate (26.7 mL, 1.1 eq), while the temperature was maintained at 20° C. The reaction mixture was agitated for 16 hrs until complete by HPLC, then concentrated to dryness to afford 5 (71.7 g, quant.) and used directly in the next reaction. C₁₁H₁₄BrN₃O₃S (calc M+H: 348.0012), LCMS (EI) m/e 348.1 (M⁺+H).

### Intermediate (6). 7-bromo-8-ethoxy-[1,2,4]triazolo[1,5-a]pyridin-2-amine

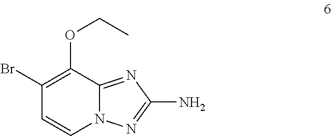

To a slurry of hydroxylamine hydrochloride (42.9 g, 3 eq) in methanol (300 mL, 4 v) was added Hünig's base (N,N-diisopropylethylamine) (71.9 mL, 2 eq) and the mixture was agitated at rt for 50 min. The reaction mixture was then added to a separate flask containing 5 (71.7 g, 1 eq) in ethanol (300 mL, 4 v) and warmed at reflux for 2 hrs until complete by HPLC. The reaction mixture was cooled to rt and concentrated to ˜1.5 v. The resultant slurry was cooled to 0° C. and quenched by the addition of ammonium hydroxide (28%, 57.3 mL, 2 eq) then diluted with water (120 mL, 2 v). The slurry was agitated 15 min, filtered, and washed with water (2×100 mL), and dried to afford 6 (48.8 g, 92% yield). ¹H NMR (400 MHz, DMSO) δ 8.25 (d, J=7.0 Hz, 1H), 7.03 (d, J=7.0 Hz, 1H), 6.16 (s, 2H), 4.55 (q, J=7.0 Hz, 2H), 1.32 (t, J=7.1 Hz, 3H). C₈H₁₀BrN₄O, (calc M+H: 257.0032), LCMS (EI) m/e 257.0 (M⁺+H).

### Intermediate (7). 2,7-dibromo-8-ethoxy-[1,2,4]triazolo[1,5-a]pyridine

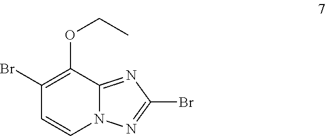

To a mixture of 6 (150 g, 583 mmol) and CuBr₂ (134 g, 600 mmol) in acetonitrile (1500 mL) at 0° C. was added t-BuONO (144.3 g, 1399 mmol) dropwise over 30 minutes. The resulting mixture was stirred at 0° C. for 2 hrs followed by 2 hrs at room temperature (TLC indicated that all of starting material was consumed). The reaction mixture was concentrated under reduced pressure. To the residue was added DCM (3 L), and stirred for half an hour. The mixture was then passed through a pad of silica gel (300 g), eluting with DCM until no product was detected by TLC. The fraction collected was evaporated to afford the desired product (137 g). To the product (137 g) in EtOAc (1500 mL) was added charcoal (45 g, ˜100 mesh particle size), and the resulting suspension was stirred at room temperature for 2 hrs. The mixture was filtered through a pad of Celite and washed with EtOAc (1500 mL). The filtrate was concentrated under reduce pressure. To the residue (125 g) was added hexanes (500 mL), and was stirred. The solid was recovered by filtration and washed with hexanes (500 mL). The filter cake was air-dried to afford 7 as a pale beige solid (111 g). ¹H NMR (400 MHz, DMSO) δ 8.65 (d, J=7.2 Hz, 1H), 7.47 (d, J=7.2 Hz, 1H), 4.65 (q, J=7.0 Hz, 2H), 1.37 (t, J=7.0 Hz, 3H). C₈H₇Br₂N₃O, (calc M+H: 319.9029), LCMS (EI) m/e 319.9 (M⁺+H).

### Example 5

### Solid State Characterization of Formula (I

### X-Ray Powder Diffraction (XRPD)

The X-Ray Powder Diffraction (XRPD) was obtained from Bruker D8 Advance ECO X-ray Powder Diffractometer (XRPD) instrument. The general experimental procedures for XRPD were: (1) X-ray radiation from copper at 1.5418 Å and LYNXEYE™ detector; (2) X-ray power at 40 kV, 25 mA; and (3) the sample powder was dispersed on a zero-background sample holder. The general measurement conditions for XRPD were: Start Angle 3 degrees; Stop Angle 30 degrees; Sampling 0.015 degrees; and Scan speed 2 degree/min.

### Differential Scanning Calorimetry (DSC)

The DSC was obtained from TA Instruments Differential Scanning Calorimetry, Discovery DSC2500 with autosampler. The DSC instrument conditions were as follows: 20-300° C. at 10° C./min; Tzero aluminum sample pan and lid; and nitrogen gas flow at 50 mL/min.

### Thermogravimetric Analysis (TGA)

The TGA was obtained from TA Instruments Thermogravimetric Analyzer, Discovery TGA5500 with autosampler. The general experimental conditions for TGA were: ramp from 25° C. to 300° C. at 10° C./min; nitrogen purge gas flow at 25 mL/min; platinum sample holder.

### Formula (I) Free Base Form I

The crystalline free base of the compound of Formula (I) as obtained in Examples 1 and 2 was characterized and is referred to herein as Form I. Form I free base was characterized by XRPD, DSC, and TGA. The XRPD pattern is shown in FIG. 1 and FIG. 28 and the XRPD data are provided in Tables 1a and 1b, which confirms that Form I was a crystalline solid.

The DSC thermogram is shown in FIG. 2. The DSC thermogram revealed a major endothermal event at an onset temperature of 191.7° C. with a peak temperature of 193.6° C. which is believed to be the melting/decomposition temperature of the compound.

A second DSC was obtained using a Q200 V24.11 DSC using a ramp of 10° C. per minute up to 300° C. The DSC is shown in FIG. 29. The DSC thermogram revealed a major endothermal event at an onset temperature of 191.3° C. with a peak temperature of 193.3° C. which is believed to be the melting/decomposition temperature of the compound.

The TGA thermogram is shown in FIG. 3. Weight loss of 0.96% was observed at below 200° C. The compound started to decompose above 200° C.

TABLE 1a
XRPD Data for Crystalline Free Base Form I

| 2-Theta (°) | Height | H % |
|---|---|---|
| 7.1 | 3226 | 7.4 |
| 7.3 | 21873 | 50.2 |
| 7.5 | 308 | 0.7 |
| 8.0 | 236 | 0.5 |
| 8.3 | 112 | 0.3 |
| 9.7 | 236 | 0.5 |
| 10.1 | 1125 | 2.6 |
| 10.5 | 3691 | 8.5 |
| 11.2 | 349 | 0.8 |
| 11.5 | 1164 | 2.7 |
| 12.5 | 3584 | 8.2 |
| 12.8 | 35446 | 81.3 |
| 13.0 | 1610 | 3.7 |
| 13.3 | 3389 | 7.8 |
| 13.8 | 218 | 0.5 |
| 14.2 | 2284 | 5.2 |
| 14.5 | 4779 | 11.0 |
| 15.0 | 2427 | 5.6 |
| 15.2 | 7743 | 17.8 |
| 15.6 | 3290 | 7.5 |
| 16.0 | 11878 | 27.2 |
| 16.4 | 17818 | 40.9 |
| 16.6 | 1383 | 3.2 |
| 17.0 | 771 | 1.8 |
| 17.2 | 1765 | 4.0 |
| 17.4 | 2257 | 5.2 |
| 17.6 | 3220 | 7.4 |
| 17.9 | 283 | 0.7 |
| 18.0 | 513 | 1.2 |
| 18.6 | 1093 | 2.5 |
| 19.2 | 1754 | 4.0 |
| 19.5 | 586 | 1.3 |
| 19.8 | 5167 | 11.9 |
| 20.1 | 2477 | 5.7 |
| 20.3 | 43601 | 100 |
| 20.6 | 12763 | 29.3 |
| 21.3 | 36858 | 84.5 |
| 21.6 | 15085 | 34.6 |
| 21.9 | 1805 | 4.1 |
| 22.2 | 3908 | 9.0 |
| 22.6 | 3592 | 8.2 |
| 23.1 | 9060 | 20.8 |
| 23.9 | 5713 | 13.1 |
| 24.3 | 325 | 0.7 |
| 24.6 | 408 | 0.9 |
| 25.0 | 2865 | 6.6 |
| 25.7 | 4574 | 10.5 |
| 26.0 | 348 | 0.8 |
| 26.4 | 4750 | 10.9 |
| 26.8 | 7514 | 17.2 |
| 27.0 | 15041 | 34.5 |
| 27.8 | 2548 | 5.8 |
| 27.9 | 4947 | 11.3 |
| 28.2 | 213 | 0.5 |
| 28.6 | 1558 | 3.6 |
| 28.9 | 1661 | 3.8 |
| 29.2 | 1043 | 2.4 |
| 29.4 | 467 | 1.1 |
| 29.8 | 815 | 1.9 |

TABLE 1b
XRPD Data for Crystalline Free Base Form I

| 2-Theta (°) | Height | H % |
|---|---|---|
| 7.5 | 235900 | 83.2 |
| 10.6 | 30976 | 10.9 |
| 13.0 | 205684 | 72.6 |
| 14.7 | 42552 | 15.0 |
| 15.3 | 38144 | 13.5 |
| 16.2 | 121679 | 42.9 |
| 16.6 | 113132 | 39.9 |
| 17.6 | 27890 | 9.8 |
| 20.5 | 283366 | 100 |
| 20.8 | 85984 | 30.3 |
| 21.4 | 265761 | 93.8 |
| 23.3 | 71442 | 25.2 |
| 24.0 | 53028 | 18.7 |
| 25.2 | 29928 | 10.6 |
| 25.9 | 38144 | 13.5 |
| 27.1 | 136962 | 48.3 |
| 28.0 | 64762 | 22.8 |
| 28.9 | 29217 | 10.3 |
| 30.5 | 12495 | 4.4 |
| 32.8 | 14303 | 5.0 |
| 37.5 | 10129 | 3.6 |
| 39.4 | 2725 | 1.0 |
| 40.3 | 8331 | 29.4 |

### Formula (I) Maleate Salt

### Preparation of Formula (I) Maleate Salt

574.0 mg of free base was dissolved in 8 mL of 1:1 dichloromethane (DCM)/methanol in a 20 mL clear glass vial with stirring. To the solution, 191.3 mg of maleic acid (1.2 eq) was added and mixed well. The solution was evaporated without a cap at room temperature to dryness overnight. To the resulting solid, 5 mL of acetone was added and stirred for 2 hrs at room temperature. The solid was collected by filtration, washed with acetone and vacuum dried at 50° C. for 2 hrs. The salt ratio between the free base and maleic acid was determined to be 1.0 by NMR analysis.

The maleate salt was confirmed as a crystalline solid according to XRPD analysis. The XRPD pattern is shown in FIG. 4 and the peak data are provided in Table 1.

The DSC thermogram is shown in FIG. 5. The DSC thermogram revealed a major endothermal event at an onset temperature of 180.4° C. with a peak temperature of 181.9° C. which is believed to be the melting/decomposition temperature of the compound.

The TGA thermogram is shown in FIG. 6. Weight loss of 19.9% was observed between 100-260° C.

TABLE 2
XRPD Data for Maleate Salt

| 2-Theta (°) | Height | H % |
|---|---|---|
| 8.5 | 145 | 0.8 |
| 10.4 | 11653 | 66.2 |
| 11.6 | 4629 | 26.3 |
| 12.0 | 17607 | 100 |
| 14.1 | 6311 | 35.8 |
| 14.4 | 1058 | 6.0 |
| 14.9 | 3931 | 22.3 |
| 15.1 | 5917 | 33.6 |
| 16.3 | 1769 | 10.0 |
| 16.6 | 297 | 1.7 |
| 17.2 | 8252 | 46.9 |
| 17.4 | 736 | 4.2 |
| 18.1 | 4947 | 28.1 |
| 19.1 | 11887 | 67.5 |
| 19.7 | 4533 | 25.7 |
| 20.0 | 3436 | 19.5 |
| 20.2 | 4927 | 28.0 |
| 20.9 | 6665 | 37.9 |
| 21.2 | 4482 | 25.5 |
| 21.3 | 8857 | 50.3 |
| 21.5 | 5010 | 28.5 |
| 21.9 | 10453 | 59.4 |
| 22.3 | 151 | 0.9 |
| 22.9 | 8359 | 47.5 |
| 23.5 | 1830 | 10.4 |
| 23.8 | 376 | 2.1 |
| 24.2 | 9029 | 51.3 |
| 24.9 | 1581 | 9.0 |
| 25.2 | 3574 | 20.3 |
| 25.9 | 6318 | 35.9 |
| 26.5 | 369 | 2.1 |
| 26.9 | 813 | 4.6 |
| 27.0 | 730 | 4.1 |
| 27.6 | 1693 | 9.6 |
| 28.1 | 927 | 5.3 |
| 28.4 | 1539 | 8.7 |
| 28.9 | 1018 | 5.8 |
| 29.1 | 3192 | 18.1 |
| 29.7 | 429 | 2.4 |

### Formula (I) Besylate Salt

### Preparation of Formula (I) Besylate Salt

104.98 mg of the free base of Formula (I) was dissolved in 2 mL of 1:1 dichloromethane (DCM)/methanol in a 4 mL clear glass vial with stirring. To the solution, 47.75 mg of benzenesulfonic acid (1.2 eq) was added and mixed well. The solution was evaporated without a cap at room temperature to oil overnight. To the resulting oil, 1 mL of acetonitrile was added to obtain a solution with stirring at room temperature. The solution was evaporated again without a cap at room temperature to oil overnight. Then 1 mL of acetone was added to the oil and slurried to solid for 1-2 hrs at room temperature. The besylate salt was collected by filtration, washed with acetone, and vacuum dried at 50° C. for 1 hr. The salt ratio between the free base and benzenesulfonic acid was determined to be 2.0 by NMR analysis.

The besylate salt was confirmed as a crystalline solid according to XRPD analysis. The XRPD pattern is shown in FIG. 7 and the peak data are provided in Table 3.

The DSC thermogram is shown in FIG. 8. The DSC thermogram revealed a major endothermal event at an onset temperature of 160.4° C. with a peak temperature of 163.4° C. which is believed to be the melting/decomposition temperature of the compound.

The TGA thermogram is shown in FIG. 9. Weight loss of 1.0% was observed below 150° C. and significant weight loss occurred above 150° C. due to decomposition of the compound.

TABLE 3
XRPD Data for Besylate Salt

| 2-Theta (°) | Height | H % |
|---|---|---|
| 6.3 | 45298 | 100 |
| 8.4 | 240 | 0.5 |
| 9.9 | 2592 | 5.7 |
| 11.6 | 567 | 1.3 |
| 11.9 | 2520 | 5.6 |
| 12.1 | 5502 | 12.1 |
| 12.6 | 3707 | 8.2 |
| 13.3 | 1286 | 2.8 |
| 14.2 | 701 | 1.5 |
| 14.8 | 161 | 0.4 |
| 15.5 | 766 | 1.7 |
| 15.9 | 2920 | 6.4 |
| 16.3 | 279 | 0.6 |
| 16.5 | 856 | 1.9 |
| 17.0 | 789 | 1.7 |
| 17.4 | 3847 | 8.5 |
| 18.0 | 351 | 0.8 |
| 18.4 | 3569 | 7.9 |
| 18.7 | 3638 | 8.0 |
| 19.0 | 8399 | 18.5 |
| 19.6 | 4324 | 9.5 |
| 19.9 | 643 | 1.4 |
| 20.6 | 533 | 1.2 |
| 20.8 | 1392 | 3.1 |
| 21.1 | 782 | 1.7 |
| 21.6 | 2270 | 5.0 |
| 22.2 | 3312 | 7.3 |
| 23.1 | 781 | 1.7 |
| 23.3 | 2880 | 6.4 |
| 23.7 | 3021 | 6.7 |
| 24.1 | 1640 | 3.6 |
| 24.4 | 577 | 1.3 |
| 25.1 | 4225 | 9.3 |
| 25.3 | 1078 | 2.4 |
| 26.0 | 2419 | 5.3 |
| 26.9 | 1317 | 2.9 |
| 28.0 | 991 | 2.2 |
| 28.7 | 994 | 2.2 |
| 29.2 | 210 | 0.5 |

### Formula (I) Mesylate Salt

### Preparation of Formula (I) Mesylate Salt

108.57 mg of the free base of Formula (I) was dissolved in 2 mL of 1:1 dichloromethane (DCM)/methanol in a 4 mL clear glass vial with stirring. To the solution, 20.2 μL of methanesulfonic acid (1.2 eq) was added and mixed well. The solution was evaporated without a cap at room temperature to oil overnight. To the resulting oil, 1 mL of acetone was added and slurried to solid for 1 hr at room temperature. The mesylate salt was collected by filtration, washed with acetone, and vacuum dried at 50° C. for 1 hr. The salt ratio between the free base and methanesulfonic acid was determined to be 1.2 by NMR analysis.

The mesylate salt was confirmed as a crystalline solid according to XRPD analysis. The XRPD pattern is shown in FIG. 10 and the peak data are provided in Table 4.

The DSC thermogram is shown in FIG. 11. The DSC thermogram revealed first dehydration at an onset temperature of 24.6° C. with a peak temperature of 61.1° C. and a second endothermal event at an onset temperature of 134.4° C. with a peak temperature of 150.1° C. which is believed to be the melting/decomposition temperature of the compound.

The TGA thermogram is shown in FIG. 12. Weight loss of 0.65% was observed below 125° C. and significant weight loss occurred above 150° C. due to decomposition of the compound.

TABLE 4
XRPD Data for Mesylate Salt

| 2-Theta (°) | Height | H % |
|---|---|---|
| 4.8 | 3485 | 21.3 |
| 7.0 | 8253 | 50.4 |
| 8.6 | 699 | 4.3 |
| 9.7 | 106 | 0.6 |
| 10.6 | 120 | 0.7 |
| 11.9 | 16384 | 100 |
| 12.9 | 261 | 1.6 |
| 14.1 | 4036 | 24.6 |
| 14.6 | 2349 | 14.3 |
| 14.9 | 4916 | 30.0 |
| 16.1 | 718 | 4.4 |
| 16.2 | 477 | 2.9 |
| 17.0 | 679 | 4.1 |
| 17.3 | 307 | 1.9 |
| 17.7 | 3073 | 18.8 |
| 18.4 | 1297 | 7.9 |
| 18.9 | 4903 | 29.9 |
| 19.4 | 284 | 1.7 |
| 20.2 | 7937 | 48.4 |
| 20.8 | 619 | 3.8 |
| 21.3 | 164 | 1.0 |
| 21.9 | 3217 | 19.6 |
| 22.1 | 7682 | 46.9 |
| 23.0 | 3101 | 18.9 |
| 23.7 | 2805 | 17.1 |
| 24.3 | 2003 | 12.2 |
| 25.3 | 683 | 4.2 |
| 26.1 | 4839 | 29.5 |
| 26.3 | 1070 | 6.5 |
| 26.9 | 108 | 0.7 |
| 27.3 | 833 | 5.1 |
| 28.2 | 151 | 0.9 |
| 28.5 | 1374 | 8.4 |
| 28.8 | 695 | 4.2 |
| 29.1 | 736 | 4.5 |
| 29.4 | 443 | 2.7 |

### Formula (I) Tosylate Salt

### Preparation of Formula (I) Tosylate Salt

121.81 mg of the free base of Formula (I) was dissolved in 2 mL of 1:1 dichloromethane (DCM)/methanol in a 4 mL clear glass vial with stirring. To the solution, 67.03 mg of p-toluenesulfonic acid monohydrate (1.2 eq) was added and mixed well. The solution was evaporated without a cap at room temperature to oil overnight. To the resulting oil, 1 mL of acetonitrile was added to obtain a solution with stirring at room temperature. The solution was evaporated again without a cap at room temperature to oil/semi-solid overnight. Then 1 mL of acetone was added and slurried to solid for 1 hr at room temperature. The tosylate salt was collected by filtration, washed with acetone, and vacuum dried at 50° C. for 1 hr. The salt ratio between the free base and toluenesulfonic acid was determined to be 2.0 by NMR analysis.

The tosylate salt was confirmed as a crystalline solid according to XRPD analysis. The XRPD pattern is shown in FIG. 13 and the peak data are provided in Table 5.

The DSC thermogram is shown in FIG. 14. The DSC thermogram revealed one exothermal event at an onset temperature of 99.6° C. with a peak temperature of 110.5° C. and one endothermal event at an onset temperature of 216.1° C. with a peak temperature of 218.7° C. which is believed to be the melting/decomposition temperature of the compound.

The TGA thermogram is shown in FIG. 15. Weight loss of 0.76% was observed below 150° C. and significant weight loss occurred above 200° C. due to decomposition of the compound.

TABLE 5
XRPD Data for Tosylate Salt

| 2-Theta (°) | Height | H % |
|---|---|---|
| 5.7 | 3037 | 42.4 |
| 5.9 | 855 | 11.9 |
| 7.8 | 7161 | 100 |
| 8.1 | 1663 | 23.2 |
| 8.4 | 557 | 7.8 |
| 8.9 | 645 | 9.0 |
| 9.3 | 1691 | 23.6 |
| 9.9 | 329 | 4.6 |
| 10.2 | 351 | 4.9 |
| 11.3 | 1108 | 15.5 |
| 11.9 | 793 | 11.1 |
| 12.5 | 874 | 12.2 |
| 13.5 | 733 | 10.2 |
| 13.7 | 1711 | 23.9 |
| 13.9 | 2254 | 31.5 |
| 14.5 | 1316 | 18.4 |
| 15.7 | 132 | 1.8 |
| 16.2 | 1961 | 27.4 |
| 16.5 | 663 | 9.3 |
| 16.9 | 1586 | 22.1 |
| 17.1 | 1466 | 20.5 |
| 17.5 | 1318 | 18.4 |
| 17.8 | 409 | 5.7 |
| 18.3 | 1261 | 17.6 |
| 18.8 | 1841 | 25.7 |
| 19.5 | 383 | 5.3 |
| 20.3 | 1631 | 22.8 |
| 20.6 | 2753 | 38.4 |
| 21.0 | 1567 | 21.9 |
| 21.5 | 1034 | 14.4 |
| 22.3 | 874 | 12.2 |
| 23.0 | 703 | 9.8 |
| 23.3 | 522 | 7.3 |
| 24.0 | 633 | 8.8 |
| 24.5 | 1652 | 23.1 |
| 24.8 | 1506 | 21.0 |
| 25.8 | 289 | 4.0 |
| 26.3 | 118 | 1.6 |
| 27.0 | 316 | 4.4 |
| 27.2 | 421 | 5.9 |
| 27.7 | 120 | 1.7 |
| 28.6 | 493 | 6.9 |
| 28.8 | 511 | 7.1 |

### Formula (I) Mono-Hydrochloride Salt

### Preparation of Formula (I) Mono-Hydrochloride Salt

102.7 mg of the free base of Formula (I) was dissolved in 2 mL of 1:1 dichloromethane (DCM)/methanol in a 4 mL clear glass vial with stirring. To the solution, 49 μL of 6 M aqueous hydrochloric acid (1.2 eq) was added and mixed well. The solution was evaporated without a cap at room temperature to dry solid overnight. To the resulting solid, 1 mL of acetone was added and slurried for 1 hr at room temperature. The mono-hydrochloride salt was collected by filtration, washed with acetone and vacuum dried at 50° C. for 1 hr. The salt ratio between the free base and hydrochloric acid was determined to be 1.08 by ion chromatography analysis.

The mono-hydrochloride salt was confirmed as a crystalline solid according to XRPD analysis. The XRPD pattern is shown in FIG. 16 and the peak data are provided in Table 6.

The DSC thermogram is shown in FIG. 17. The DSC thermogram revealed one major endothermal event at an onset temperature of 196.0° C. with a peak temperature of 212.2° C. which is believed to be the melting/decomposition temperature of the compound.

The TGA thermogram is shown in FIG. 18. Weight loss of 9.4% was observed below 225° C. Weight loss continued above 225° C. due to decomposition of the compound.

TABLE 6
XRPD Data for Mono-Hydrochloride Salt

| 2-Theta (°) | Height | H % |
|---|---|---|
| 5.7 | 668 | 41.3 |
| 8.5 | 944 | 58.4 |
| 10.8 | 149 | 9.2 |
| 11.3 | 320 | 19.8 |
| 13.0 | 507 | 31.4 |
| 13.8 | 1333 | 82.5 |
| 14.1 | 1535 | 95.0 |
| 15.0 | 1483 | 91.8 |
| 15.1 | 1330 | 82.3 |
| 15.7 | 639 | 39.6 |
| 17.1 | 134 | 8.3 |
| 18.4 | 1268 | 78.5 |
| 19.3 | 1108 | 68.6 |
| 19.9 | 261 | 16.2 |
| 20.5 | 411 | 25.5 |
| 21.8 | 836 | 51.8 |
| 22.1 | 784 | 48.5 |
| 22.8 | 596 | 36.9 |
| 23.7 | 126 | 7.8 |
| 24.0 | 332 | 20.6 |
| 24.8 | 358 | 22.2 |
| 25.7 | 1616 | 100 |
| 28.8 | 244 | 15.1 |
| 29.2 | 146 | 9.0 |

### Formula (I) Di-Hydrochloride Salt

### Preparation of Formula (I) Di-Hydrochloride Salt

100.35 mg of the free base of Formula (I) was dissolved in 2 mL of 1:1 dichloromethane (DCM)/methanol in a 4 mL clear glass vial with stirring. To the solution, 88 μL of 6 M aqueous hydrochloric acid (2.2 eq) was added and mixed well. The solution was evaporated without a cap at room temperature to 0.2-0.3 mL with solid overnight. Then 1 mL of acetone was added and slurried for 1 hr at room temperature. The di-hydrochloride salt was collected by filtration, washed with acetone, and vacuum dried at 50° C. for 1 hr. The salt ratio between the free base and hydrochloric acid was determined to be 1.50 by ion chromatography analysis.

The di-hydrochloride salt was confirmed as a crystalline solid according to XRPD analysis. The XRPD pattern is shown in FIG. 19 and the peak data are provided in Table 7.

The DSC thermogram is shown in FIG. 20. The DSC thermogram revealed one major endothermal event at an onset temperature of 182.1° C. with a peak temperature of 206.4° C. which is believed to be the melting/decomposition temperature of the compound.

The TGA thermogram is shown in FIG. 21. Weight loss was observed in multiple steps: 8.3% below 175° C. and 7.3% between 170° C. and 240° C. Weight loss continued above 240° C. due to decomposition of the compound.

TABLE 7
XRPD Data for Di-Hydrochloride Salt

| 2-Theta (°) | Height | H % |
|---|---|---|
| 7.2 | 515 | 4.3 |
| 8.4 | 235 | 2.0 |
| 9.9 | 792 | 6.6 |
| 10.7 | 761 | 6.4 |
| 12.3 | 1046 | 8.8 |
| 12.5 | 210 | 1.8 |
| 13.0 | 7663 | 64.1 |
| 13.4 | 1204 | 10.1 |
| 14.0 | 6209 | 52.0 |
| 14.5 | 186 | 1.6 |
| 15.2 | 3901 | 32.7 |
| 15.7 | 1789 | 15.0 |
| 16.0 | 498 | 4.2 |
| 16.5 | 56 | 0.5 |
| 16.9 | 723 | 6.1 |
| 17.4 | 534 | 4.5 |
| 17.6 | 1838 | 15.4 |
| 18.2 | 1535 | 12.9 |
| 18.6 | 761 | 6.4 |
| 18.8 | 684 | 5.7 |
| 19.6 | 2681 | 22.4 |
| 19.9 | 5945 | 49.8 |
| 20.7 | 484 | 4.1 |
| 21.3 | 254 | 2.1 |
| 21.8 | 11946 | 100 |
| 22.3 | 2797 | 23.4 |
| 23.2 | 1109 | 9.3 |
| 23.3 | 1352 | 11.3 |
| 23.7 | 1655 | 13.9 |
| 24.0 | 3459 | 29.0 |
| 24.4 | 523 | 4.4 |
| 24.8 | 7162 | 60.0 |
| 25.1 | 1437 | 12.0 |
| 25.5 | 914 | 7.7 |
| 25.6 | 1056 | 8.8 |
| 26.2 | 1731 | 14.5 |
| 26.5 | 549 | 4.6 |
| 26.8 | 906 | 7.6 |
| 27.7 | 219 | 1.8 |
| 28.3 | 250 | 2.1 |
| 28.8 | 2439 | 20.4 |
| 29.0 | 1680 | 14.1 |
| 29.3 | 1233 | 10.3 |
| 29.5 | 712 | 6.0 |

### Example 6

### Formula (I) Studies

### Solubility Measurement

The solubility of the compound of Formula (I) Form I was measured at 25° C. at 50° C.

The solubility measurement at 25° C. was performed according to the following procedure. 5 mL of solvent (see Table 8) was added to the individual vials. The compound of Formula (I) Form I was added to the vials to get a cloudy solution at 25° C. Another approximately 20 mg of the compound of Formula (I) Form I was added to the cloudy solution. The mixture was agitated at 25±1° C. for 48 h, which was controlled by IKA® ETS-D5 temperature controller and IKA® RCT basic safety control. The supernatant was filtered using a syringe filter (0.22 μm). The saturated solution was pipetted into HPLC vials and diluted with MeOH or acetone. The samples were analyzed by HPLC and the corresponding solubility was calculated as indicated in Table 8.

The solubility measurement at 50° C. was performed according to the following procedure. 5 mL of solvent (see Table 8) was added to the individual vials. The compound of Formula (I) Form I was added to the vials to get a cloudy solution at 50° C. Another approximately 20-25 mg of the compound of Formula (I) Form I was added. The mixture was agitated at 50±1° C. for 24 h, which was controlled by IKA® ETS-D5 temperature controller and IKA® RCT basic safety control. The supernatant was filtered quickly using a warmed syringe filter (0.22 μm) at 50±1° C. The saturated solution was pipetted into HPLC vials and diluted with MeOH or acetone. The samples were analyzed by HPLC and the corresponding solubility was calculated as indicated in Table 8.

TABLE 8
Solubility (mg/mL) of the compound of
Formula (I) Form I in various solvents

| | Solubility | Solubility |
|---|---|---|
| Solvent | (25° C.) | (50° C.) |
| MeCN | 7.5 | 30.1 |
| Chloroform | >50* | >50* |
| Dichloromethane | 18.5 | 37.1 |
| Dimethylformamide | >50* | >50* |
| 1,4-Dioxane | >50* | >50* |
| MeOH | 18.7 | 48.1 |
| 2-Methoxyethanol | >50* | >50* |
| Methyl iso-butyl ketone | 2.5 | 4.5 |
| Toluene | 0.1 | 0.5 |
| Acetone | 30.1 | 40.2 |
| n-BuOH | 3.9 | 9.7 |
| tert-Butyl methyl ether | 0.2 | 0.3 |
| DMSO | >50* | >50* |
| EtOH | 6.8 | 15.7 |
| EtOAc | 3.8 | 6.8 |
| Ethyl formate | 11.8 | 15.2 |
| Heptane | 0 | 0 |
| iso-Butyl acetate | 1.1 | 1.6 |
| iso-Propyl acetate | 1.8 | 3.0 |
| n-Propanol | 4.8 | 13.1 |
| iso-Propanol (IPA) | 3.3 | 9.1 |
| Water | 0.06 | 0.2 |
| Methyl ethyl ketone | 15.9 | 25.3 |
| Tetrahydrofuran (THF) | >50* | >50* |
| 2-Methyl THF | 9.6 | 18.7 |
| 2-Me THF/H₂O/Heptane (1:1.5%:1) | 0.2 | 0.6 |
| 2-Me THF/H₂O/Heptane (1:3%:1) | 0.3 | 0.4 |
| 3% water in EtOH | 9.8 | 20.0 |
| 6% water in EtOH | 14.9 | 36.4 |

At 25° C., the compound of Formula (I) Form I had excellent solubility in (>50 mg/mL) in CHCl₃, dimethylformamide (DMF), 1,4-dioxane, 2-methoxyethanol, DMSO, and THF. It had relatively good solubility (15 mg/mL<solubility<50 mg/mL) in dichloromethane, MeOH, acetone, methyl ethyl ketone. It was slightly soluble (1 mg/mL<solubility<15 mg/mL) in acetonitrile, methyl iso-butyl ketone, EtOH, n-propanol, iso-propanol, n-butanol, EtOAc, iso-propyl acetate, iso-butyl acetate, ethyl formate, 2-methyl THF, 3% water in EtOH, and 6% water in EtOH. It had poor solubility (<1 mg/mL) in toluene, tert-butyl methyl ether (MTBE), water, 2-methyl THF/H₂O/Heptane (volume ratio 1:1.5%:1), and 2-methyl THF/H₂O/Heptane (volume ratio 1:3%:1). It was completely insoluble in heptane.

At 50° C., the compound of Formula (I) Form I had excellent solubility in (>50 mg/mL) in CHCl₃, dimethylformamide (DMF), 1,4-dioxane, 2-methoxyethanol, DMSO, and THF. It had relatively good solubility (15 mg/mL<solubility<50 mg/mL) in acetonitrile, dichloromethane, MeOH, EtOH, acetone, methyl ethyl ketone, ethyl formate, 2-methyl THF, 3% water in EtOH, and 6% water in EtOH. It was slightly soluble (1 mg/mL<solubility<15 mg/mL) in methyl iso-butyl ketone, n-propanol, iso-propanol, n-butanol, EtOAc, iso-propyl acetate, and iso-butyl acetate. It had poor solubility (<1 mg/mL) in toluene, tert-butyl methyl ether (MTBE), water, 2-methyl THF/H₂O/Heptane (volume ratio 1:1.5%:1), and 2-methyl THF/H₂O/Heptane (volume ratio 1:3%:1). It was completely insoluble in heptane.

### Phase Equilibrium

Phase equilibration studies were conducted to identify a predominant crystal form for phase identification. Based on its solubility in various solvent systems (Table 8), the compound of Formula (I) Form I was equilibrated in a representative groups of solvents at 25±1° C. and 50±1° C. To the solvents, the compound of Formula (I) Form I was added until a cloudy solution was obtained, then approximately 20 mg of the compound of Formula (I) Form I was added to the cloudy solution. The mixture was stirred at 25±1° C. or 50±1° C. for 48 hours or 24 hours, respectively. The solid was filtered, dried in vacuum, and analyzed by XRPD.

The material obtained showed as crystalline Form I for phase equilibration at 25° C. and 50° C. in all solvents tested. The results are shown in Table 9 (N/A indicates not tested).

TABLE 9
Crystalline form for phase equilibration

| | Solid Form | Solid Form |
|---|---|---|
| Solvent | (25° C.) | (50° C.) |
| MeCN | I | I |
| Chloroform | I | N/A |
| DCM | I | I |
| MeOH | I | I |
| MIBK | I | I |
| Toluene | I | I |
| Acetone | I | I |
| n-BuOH | I | I |
| MTBE | I | I |
| EtOH | I | I |
| EtOAc | I | I |
| Ethyl formate | I | I |
| Heptane | I | I |
| Isobutyl acetate | I | I |
| IPAc | I | I |
| n-Propanol | I | I |
| IPA | I | I |
| Water | I | I |
| MEK | I | I |
| THF | I | N/A |
| 2-Methyl THF | I | I |
| Me-THF/H₂O/Heptane (1:0.015:1) | I | I |
| Me-THF/H₂O/Heptane (1:0.03:1) | I | I |
| 3% H₂O in EtOH | I | I |
| 6% H₂O in EtOH | I | I |

### Evaporation Studies

Evaporation studies were carried out to identify the predominant crystalline form during uncontrolled precipitation. XRPD was used to study the solid-state morphology of the crystalline forms of the evaporation samples at 25±1° C. and 50±1° C. The results are shown in Table 10 (N/A indicates that the amount of precipate was too small to be analyzed by XRPD).

TABLE 10
Crystalline form from evaporation

| | Solid Form | Solid Form |
|---|---|---|
| Solvent | (25° C.) | (50° C.) |
| MeCN | II | I + II |
| Chloroform | N/A | N/A |
| DCM | II | I + II |
| DMF | N/A | N/A |
| 1,4-Dioxane | III | N/A |
| MeOH | I | I |
| 2-Methoxy-ethanol | N/A | N/A |
| MIBK | I | I |
| Toluene | N/A | N/A |
| Acetone | I | I |
| n-BuOH | I | I |
| MTBE | N/A | N/A |
| DMSO | N/A | N/A |
| EtOH | II | I |
| EtOAc | I | I |
| Ethyl formate | I | I |
| Heptane | N/A | N/A |
| Isobutyl acetate | I | I |
| IPAc | I | I |
| n-Propanol | I + II | I |
| IPA | II | I |
| Water | N/A | N/A |
| MEK | I | I |
| THF | N/A | N/A |
| 2-Methyl THF | I | I |
| Me-THF/H₂O/Heptane (1:0.015:1) | I | I |
| Me-THF/H₂O/Heptane (1:0.03:1) | I | I |
| 3% H₂O in EtOH | I | I |
| 6% H₂O in EtOH | I | I |

The solids of the compound of Formula (I) obtained after evaporation at 25° C. in dichloromethane, acetonitrile, ethanol, and IPA had a different XRPD pattern when compared to that of the staring material (the compound of Formula (I) Form I) and is referred to as Form II. The solids obtained after evaporation at 25° C. in 1,4-dioxane had a different XRPD pattern when compared to those of Form I and Form II and is referred to as Form III.

### Anti-Solvent Addition

Saturated solutions and nearly saturated solutions of the compound of Formula (I) Form I were prepared in the solvents listed in Table 11 at room temperature. An anti-solvent was added dropwise to induce precipitation. As indicated in Table 11, only Form I was observed.

TABLE 11
Precipitation from anti-solvent addition

| Solvent (mL) | Anti-solvent (mL) | Solid Form |
|---|---|---|
| DMF (1.0) | MTBE (10) | I |
| DMF (1.0) | H₂O (10) | I |
| 1,4-Dioxane (1.0) | MTBE (10) | I |
| 1,4-Dioxane (1.0) | H₂O (10) | I |
| 2-Methoxy-ethanol (1.0) | MTBE (10) | I |
| 2-Methoxy-ethanol (1.0) | H₂O (10) | I |
| THF (1.0) | MTBE | I |
| THF (1.0) | H₂O (10) | I |

### Reverse Addition

Saturated solutions and nearly saturated solutions of the compound of Formula (I) Form I were prepared in the solvents listed in Table 12 at 25° C. and added dropwise to a larger volume of a miscible anti-solvent. As shown in Table 12, in the reverse addition experiments, no new polymorphic forms were identified.

TABLE 12
Precipitation from reverse addition

| Solvent (mL) | Anti-solvent (mL) | Solid Form |
|---|---|---|
| DMF (1.0) | MTBE (8) | I |
| DMF (1.0) | H₂O (8) | I |
| 1,4-Dioxane (1.0) | MTBE (8) | I |
| 1,4-Dioxane (1.0) | H₂O (8) | I |
| 2-Methoxy-ethanol (1.0) | MTBE (8) | I |
| 2-Methoxy-ethanol (1.0) | H₂O (8) | I |
| THF (1.0) | MTBE (8) | I |
| THF (1.0) | H₂O(8) | I |

### Quench Cool of Saturated Solution

Saturated or nearly saturated solutions of the compound of Formula (I) prepared at 25° C. were quench-cooled to about −20 to about −30° C. to induce precipitation of higher energy forms. Representative solvents were chosen based on solubility data measured at 25° C. and 50° C. As shown in Table 13, Form III solids were observed in 1,4-dioxane.

TABLE 13
Crystal forms from quench cool experiment

| Solvent | Solid Form |
|---|---|
| DMF | N/A (sticky) |
| 1,4-Dioxane | III |
| MeOH | I |
| 2-Methoxy-ethanol | N/A (sticky) |
| THF | I |

Crystallization of Saturated Solutions with Heating and Cooling Cycles

This experiment was conducted to identify a more stable form of the compound of Formula (I) than Form I. Saturated solutions of the compound of Formula (I) were prepared at 50° C. and cooled in a bath slowly by using a programmed circulating bath. To the clear solution was added about 10 mg of the compound of Formula (I) Form I to give a slurry. The formed slurry was then heated at 50° C. over 2 hours and then cooled down to 5° C. over 2 hours. This process was repeated for 3 days and the solid was filtered for further analysis. The solvents used were 1,4-dioxane, methanol, or THF. The experiments only resulted in Form I.

### Stability Relationship

The relative stability of the three forms (Forms I-III) of the compound of Formula (I) was studied and compared by phase equilibration of mixture experiments in three solvent system (3% water [volume ratio] in EtOH using 3 volumes of solvent, 3% water in EtOH using 5 volumes of solvent, and 6% water in EtOH using 5 volumes of solvent). The three crystal forms were mixed and slurried at 50° C. for more than 6 hours. The mixtures of the three forms were transformed to Form I in the three specific solvents after the six-hour-stirring competitive slurry.

### Example 7

### Characterization of Form II

The compound of Formula (I) Form II was prepared by evaporation at 25° C. in CH₂Cl₂, CH₃CN, EtOH, and TPA (see Example 6, Table 10). Form II (obtained in CH₂Cl₂) was characterized by HPLC, ¹H NMR, XRPD, DSC, and TGA. The XRPD pattern is shown in FIG. 22 and the XRPD data are provided in Table 14.

The DSC thermogram is shown in FIG. 23. When comparing the DSC of Form I and Form II, it was observed that Form II solids contained two peaks (FIG. 23), with the latter peak onset (≈191.0° C.) and Tₘₐₓ (193.4° C.) similar to the peak onset and Tₘₐₓ of Form I (FIG. 2). Form II changed to Form I during the heating process (see Example 6).

The TGA thermogram in shown in FIG. 24.

TABLE 14
XRPD Data from Form III

| 2-Theta (°) | Height | H % |
|---|---|---|
| 5.8 | 29295 | 17.9 |
| 7.6 | 42830 | 26.1 |
| 11.4 | 29838 | 18.2 |
| 12.5 | 163864 | 100 |
| 14.4 | 41540 | 25.4 |
| 16.2 | 9896 | 6.0 |
| 17.2 | 21335 | 13.0 |
| 17.9 | 47812 | 29.2 |
| 20.4 | 12664 | 7.7 |
| 21.4 | 13831 | 8.4 |
| 23.3 | 2113 | 1.3 |
| 25.3 | 34434 | 21.0 |
| 27.1 | 10735 | 6.6 |
| 28.0 | 697 | 0.4 |

### Example 8

### Characterization of Form III

The compound of Formula (I) Form III was prepared by evaporation at 25° C. in 1,4-dioxane (see Example 6, Table 10) and the quench cool experiment in 1,4-dioxane (see Example 6, Table 13). Form III was characterized by HPLC, ¹H NMR, XRPD, DSC, and TGA. The XRPD pattern is shown in FIG. 25 and the XRPD data are provided in Table 15.

Quantitative ¹H NMR exhibited that the residual 1,4-dioxane in the solids (wt %) was approximately 4.9800. The residual solvent could not be removed by conventional drying methods. This suggested that the solids obtained were potentially a solvate, supported by TGA experiments (up to 5.000% weight loss up to 180° C.; FIG. 27). The stoichiometric ratio of the compound of Formula (I) and 1,4-dioxane could be 4:1. The DSC of the solid (FIG. 26) contained a peak onset latter peak onset (≈192.6° C.) and Tₘₐₓ (194.3° C.), which are similar to the peak onset (191.3° C.) and Tₘₐₓ (193.3° C.) of Form I (FIG. 2). Form III changed to Form I during the heating process (see Example 6).

TABLE 15
XRPD Data from Form III

| 2-Theta (°) | Height | H % |
|---|---|---|
| 2.9 | 13884 | 8.7 |
| 5.5 | 73711 | 46.1 |
| 9.8 | 88136 | 55.1 |
| 10.5 | 41865 | 26.2 |
| 11.6 | 20638 | 12.9 |
| 12.1 | 35206 | 22.0 |
| 12.9 | 20612 | 12.8 |
| 13.9 | 24258 | 15.2 |
| 15.6 | 357 | 0.2 |
| 16.3 | 160045 | 100 |
| 19.1 | 18150 | 11.3 |
| 19.8 | 50056 | 31.3 |
| 22.0 | 45892 | 28.7 |
| 23.4 | 8600 | 5.4 |
| 24.4 | 91482 | 57.2 |
| 25.7 | 17399 | 10.9 |
| 27.3 | 24388 | 15.2 |
| 28.9 | 11277 | 7.0 |
| 30.6 | 7801 | 4.9 |
| 35.8 | 1441 | 0.9 |
| 39.5 | 206 | 0.1 |
| 41.7 | 8809 | 5.5 |

### Example A. CDK2/Cyclin E1 HTRF Enzyme Activity Assay

CDK2/Cyclin E1 enzyme activity assays utilize full-length human CDK2 co-expressed as N-terminal GST-tagged protein with FLAG-Cyclin E1 in a baculovirus expression system (Carna Product Number 04-165). Assays were conducted in white 384-well polystyrene plates in a final reaction volume of 8 μL. CDK2/Cyclin E1 (0.25 nM) was incubated with the compounds of the Examples (40 nL serially diluted in DMSO) in the presence of ATP (50 μM or 1 mM) and 50 nM ULight™-labeled eIF4E-binding protein 1 (THR37/46) peptide (PerkinElmer) in assay buffer (containing 50 mM HEPES pH 7.5, 1 mM EGTA, 10 mM MgCl₂, 2 mM DTT, 0.05 mg/mL BSA, and 0.01% Tween 20) for 60 minutes at room temperature. The reactions were stopped by the addition of EDTA and Europium-labeled anti-phospho-4E-BP1 antibody (PerkinElmer), for a final concentration of 15 mM and 1.5 nM, respectively. HTRF signals were read after 1 hour at room temperature on a PHERAstar FS plate reader (BMG Labtech). Data was analyzed with IDBS XLFit and GraphPad Prism 5.0 software using a three or four parameter dose response curve to determine IC₅₀ for each compound. The IC₅₀ data as measured for the compound of Formula (I) at 1 mM ATP in the assay of Example A is shown in Table 16.

TABLE 16

| Example | IC₅₀ (nM) |
|---|---|
| Compound of Formula (I) | + |

+ refers to ≤20 nM

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A solid form of a compound of Formula (I): 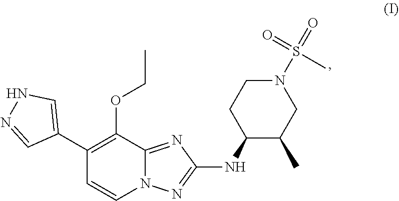 [SMILES: CCOc1c(-c2cn[nH]c2)ccn2nc(N[C@H]3CCN(S(C)(=O)=O)C[C@H]3C)nc12]
which is Form I, which is crystalline.

2. The solid form of claim 1, wherein the form has at least four XRPD peaks, in terms of 2-theta (±0.2 degrees), selected from 7.3, 10.5, 12.8, 14.5, 15.2, 16.4, 20.3, 21.3, 21.6, and 27.0.

3. The solid form of claim 1, wherein the form has at least four XRPD peaks, in terms of 2-theta (±0.2 degrees), selected from 7.5, 13.0, 14.7, 15.3, 16.2, 16.6, 20.5, 20.8, 21.4, 23.3, 24.0, and 27.1.

4. The solid form of claim 1, wherein the form has at least ten XRPD peaks, in terms of 2-theta (±0.2 degrees), selected from 7.3, 10.5, 12.8, 14.5, 15.2, 16.4, 20.3, 21.3, 21.6, and 27.0.

5. The solid form of claim 1, wherein the form has at least ten XRPD peaks, in terms of 2-theta (±0.2 degrees), selected from 7.5, 13.0, 14.7, 15.3, 16.2, 16.6, 20.5, 20.8, 21.4, 23.3, 24.0, and 27.1.

6. The solid form of claim 1, wherein the form has an XRPD pattern as substantially shown in FIG. 1.

7. The solid form of claim 1, having an endothermic peak with an onset temperature (±3° C.) at 191.7° C. and a maximum at 193.6° C.

8. The solid form of claim 1, wherein the form has a DSC thermogram substantially as shown in FIG. 2.

9. The solid form of claim 1, wherein the form has a TGA thermogram substantially as shown in FIG. 3.

10. A solid form of a compound of Formula (I): 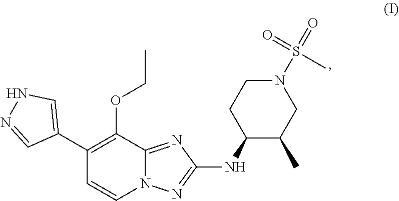 [SMILES: CCOc1c(-c2cn[nH]c2)ccn2nc(N[C@H]3CCN(S(C)(=O)=O)C[C@H]3C)nc12]
which is Form II, which is crystalline.

11. The solid form of claim 10, wherein the form has at least four XRPD peaks, in terms of 2-theta (±0.2 degrees), selected from 5.8, 7.6, 11.4, 12.5, 14.4, 17.2, 17.9, and 25.3.

12. The solid form of claim 10, wherein the form has an XRPD pattern as substantially shown in FIG. 22.

13. The solid form of claim 10, having an endothermic peak with an onset temperature (±3° C.) at 191.0° C. and a maximum at 193.4° C.

14. A solid form of a compound of Formula (I): 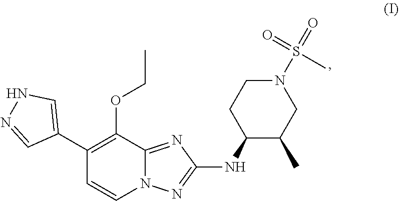 [SMILES: CCOc1c(-c2cn[nH]c2)ccn2nc(N[C@H]3CCN(S(C)(=O)=O)C[C@H]3C)nc12]
which is Form III, which is crystalline.

15. The solid form of claim 14, wherein the form has at least four XRPD peaks, in terms of 2-theta (±0.2 degrees), selected from 5.5, 9.8, 10.5, 12.1, 13.9, 16.3, 19.8, 22.0, 24.4, and 27.3.

16. The solid form of claim 14, wherein the form has an XRPD pattern as substantially shown in FIG. 25.

17. The solid form of claim 14, having an endothermic peak with an onset temperature (3° C.) at 192.6° C. and a maximum at 194.3° C.

18. A salt of a compound of Formula (I): 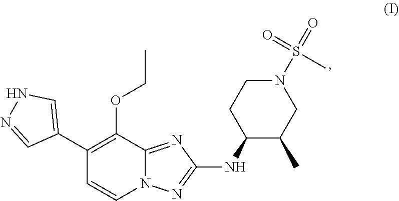 [SMILES: CCOc1c(-c2cn[nH]c2)ccn2nc(N[C@H]3CCN(S(C)(=O)=O)C[C@H]3C)nc12]
which is selected from:
a mono-maleate salt of the compound of Formula (I);
a di-besylate salt of the compound of Formula (I);
a mono-mesylate salt of the compound of Formula (I);
a di-tosylate salt of the compound of Formula (I);
a mono-hydrochloride salt of the compound of Formula (I); and
a di-hydrochloride salt of the compound of Formula (I).

19. The salt of claim 18, which is a mono-maleate salt of the compound of Formula (I).

20. The salt of claim 19, which is crystalline.

21. The salt of claim 19, wherein the salt has at least four XRPD peaks, in terms of 2-theta (±0.2 degrees), selected from 10.4, 11.6, 12.0, 14.1, 15.1, 17.2, 18.1, 19.1, 21.3, 21.9, 22.9, 24.2, and 25.9.

22. The salt of claim 19, having an endothermic peak with an onset temperature (±3° C.) at 180.4° C. and a maximum temperature (±3° C.) at 181.8° C.

23. The salt of claim 18, which is a di-besylate salt of the compound of Formula (I).

24. The salt of claim 23, which is crystalline.

25. The salt of claim 23, wherein the salt has at least four XRPD peaks, in terms of 2-theta (±0.2 degrees), selected from 6.3, 9.9, 12.1, 12.6, 15.9, 17.4, 18.7, 19.0, 19.6, and 25.1.

26. The salt of claim 23, having an endothermic peak with an onset temperature (±3° C.) at 160.4° C. and a maximum temperature (±3° C.) at 163.4° C.

27. The salt of claim 18, which is a mono-mesylate salt of the compound of Formula (I).

28. The salt of claim 27, which is crystalline.

29. The salt of claim 27, wherein the salt has at least four XRPD peaks, in terms of 2-theta (±0.2 degrees), selected from 4.8, 7.0, 11.9, 14.1, 14.9, 17.7, 18.9, 20.2, 22.1, and 26.1.

30. The salt of claim 27, having a first endothermic peak with a maximum temperature (±3° C.) at 61.1° C. and a second endothermic peak with an onset temperature (±3° C.) at 134.4° C. and a maximum temperature (±3° C.) at 150.1° C.

31. The salt of claim 18, which is a di-tosylate salt of the compound of Formula (I).

32. The salt of claim 31, which is crystalline.

33. The salt of claim 31, wherein the salt has at least four XRPD peaks, in terms of 2-theta (±0.2 degrees), selected from 5.7, 7.8, 8.1, 9.3, 13.7, 13.9, 16.2, 18.8, and 20.6.

34. The salt of claim 31, having an exothermic peak with an onset temperature (±3° C.) at 99.6° C. and a maximum temperature (±3° C.) at 110.5° C., and an endothermic peak with an onset temperature (±3° C.) at 216.1° C. and a maximum temperature (±3° C.) at 218.7° C.

35. The salt of claim 18, which is a mono-hydrochloride salt of the compound of Formula (I).

36. The salt of claim 35, which is crystalline.

37. The salt of claim 35, wherein the salt has at least four XRPD peaks, in terms of 2-theta (±0.2 degrees), selected from 5.7, 8.5, 11.3, 14.1, 15.0, 18.4, 19.3, 20.5, 21.8, 22.8, and 25.7.

38. The salt of claim 35, having an endothermic peak with an onset temperature (±3° C.) at 196.0° C. and a maximum temperature (±3° C.) at 212.2° C.

39. The salt of claim 18, which is a di-hydrochloride salt of the compound of Formula (I).

40. The salt of claim 39, which is crystalline.

41. The salt of claim 39, wherein the salt has at least four XRPD peaks, in terms of 2-theta (±0.2 degrees), selected from 9.9, 10.7, 12.3, 13.0, 14.0, 15.2, 19.9, 21.8, 22.3, and 24.8.

42. The salt of claim 39, having an endothermic peak with an onset temperature (±3° C.) at 182.1° C. and a maximum temperature (±3° C.) at 206.4° C.

43. A pharmaceutical composition comprising the solid form of claim 1, and a pharmaceutically acceptable carrier.

44. A method of inhibiting CDK2, comprising contacting the CDK2 with the solid form of claim 1.

45. A method of inhibiting CDK2 in a patient, comprising administering to the patient the solid form of claim 1.

46. A method of treating a disease or disorder associated with CDK2 in a patient, comprising administering to the patient a therapeutically effective amount of the solid form of claim 1.

47. The method of claim 46, wherein the disease or disorder is associated with an amplification of the cyclin E1 (CCNE1) gene and/or overexpression of CCNE1.

48. A method of treating a human subject having a disease or disorder associated with cyclin-dependent kinase 2 (CDK2), comprising administering to the human subject the solid form of claim 1, wherein the human subject has been previously determined to:
(i)
(a) have a nucleotide sequence encoding a p16 protein comprising the amino acid sequence of SEQ ID NO:1; and/or
(b) have a cyclin dependent kinase inhibitor 2A (CDKN2A) gene lacking one or more inactivating nucleic acid substitutions and/or deletions;
(ii)
(a) have an amplification of the cyclin E1 (CCNE1) gene; and/or
(b) have an expression level of CCNE1 in a biological sample obtained from the human subject that is higher than a control expression level of CCNE1.

49. A method of treating a human subject having a disease or disorder associated with cyclin-dependent kinase 2 (CDK2), comprising:
(i) identifying, in a biological sample obtained from the human subject:
(a) a nucleotide sequence encoding a p16 protein comprising the amino acid sequence of SEQ ID NO:1; and/or
(b) a cyclin dependent kinase inhibitor 2A (CDKN2A) gene lacking one or more inactivating nucleic acid substitutions;
(ii) identifying, in a biological sample obtained from the human subject:
(a) an amplification of the cyclin E1 (CCNE1) gene; and/or
(b) an expression level of CCNE1 that is higher than a control expression level of CCNE1; and
(iii) administering the solid form of claim 1 to the human subject.

50. The method of claim 49, comprising:
(i) identifying, in a biological sample obtained from the human subject:
(a) a nucleotide sequence encoding a p16 protein comprising the amino acid sequence of SEQ ID NO:1; and/or
(b) a CDKN2A gene lacking one or more inactivating nucleic acid substitutions and/or deletions;
(ii) identifying, in a biological sample obtained from the human subject:
(a) an amplification of the CCNE1 gene; and
(iii) administering the compound or the salt to the human subject.

51. A method of evaluating the response of a human subject having a disease or disorder associated with cyclin-dependent kinase 2 (CDK2) to the solid form of claim 1, comprising:
(a) administering the compound or the salt, to the human subject, wherein the human subject has been previously determined to have an amplification of the cyclin E1 (CCNE1) gene and/or an expression level of CCNE1 that is higher than a control expression level of CCNE1;
(b) measuring, in a biological sample of obtained from the subject subsequent to the administering of step (a), the level of retinoblastoma (Rb) protein phosphorylation at the serine corresponding to amino acid position 780 of SEQ ID NO:3,
wherein a reduced level of Rb phosphorylation at the serine corresponding to amino acid position 780 of SEQ ID NO:3, as compared to a control level of Rb phosphorylation at the serine corresponding to amino acid position 780 of SEQ ID NO:3, is indicative that the human subject responds to the compound or the salt.

52. The method of claim 46, wherein the disease or disorder is cancer.

53. A process of preparing a solid form of claim 1, comprising cooling a solution of the compound of Formula (I) in a solvent component comprising ethanol and water.

54. A process of preparing a solid form of claim 10, comprising evaporating at 25° C. a solution of the compound of Formula (I) in a solvent selected from CH₂Cl₂, CH₃CN, EtOH, and IPA.

55. A process of preparing a solid form of claim 14, comprising evaporating at 25° C. a solution of the compound of Formula (I) in 1,4-dioxane.

56. A process of preparing a compound of Formula (I), or a pharmaceutically acceptable salt thereof, comprising:
reacting a compound of Formula (1c): 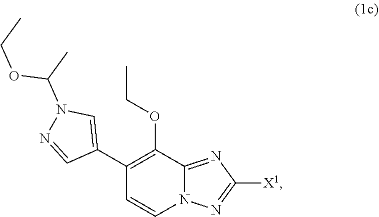
with a compound of Formula (1b): 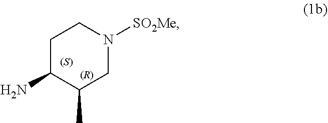
or a salt thereof, via a Buchwald coupling reaction, to form a compound of Formula (1a): 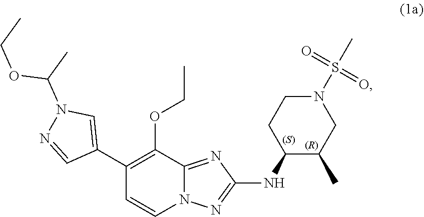
wherein X¹ is halo.

57. The process of claim 56, wherein X¹ is Br.

58. The process of claim 56, wherein the compound of formula (1b), or the salt thereof, is the HCl salt.

59. The process of claim 56, wherein the Buchwald coupling reaction comprises reacting the compound of Formula (1c) with the compound of Formula (1b), or the salt thereof, in the presence of a Buchwald catalyst or precatalyst and a base.

60. The process of claim 59, wherein the base is an alkali metal alkoxide.

61. The process of claim 56, wherein the process further comprises reacting the compound of Formula (1a) with an organic acid to form a salt of the compound of Formula (1a).

62. The process of claim 61, wherein the organic acid is succinic acid.

63. The process of claim 62, wherein the salt of the compound of Formula (1) is a hemi-succinic acid salt of the compound of Formula (1a).

64. The process of claim 56, wherein the process further comprises deprotecting the compound of Formula (1a), or a salt thereof, to form the compound of Formula (I).

65. The process of claim 56, further comprising deprotecting the compound of Formula (1a) to form the compound of Formula (I).

66. The process of claim 56, wherein the compound of Formula (1c) is prepared by a process comprising:
reacting a compound of Formula (1d): 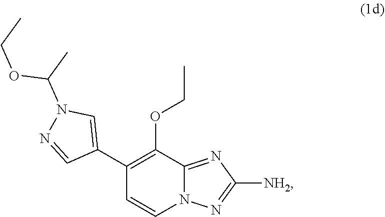 [SMILES: CCOc1c(-c2cnn(C(C)OCC)c2)ccn2nc(N)nc12]
or a salt thereof, with a halogenating agent to form the compound of formula (1c).

67. The process of claim 66, wherein the compound of Formula (1d), or the salt thereof, is prepared by a process comprising:
reacting a compound of Formula 1(e): 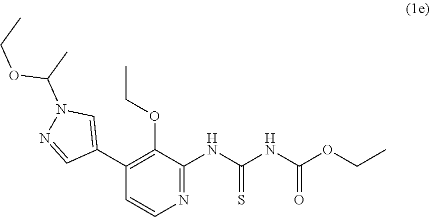 [SMILES: CCOC(=O)NC(=S)Nc1nccc(-c2cnn(C(C)OCC)c2)c1OCC]
with hydroxylamine HCl and a base component to form the compound of formula (1d), or the salt thereof.

68. A compound selected from: 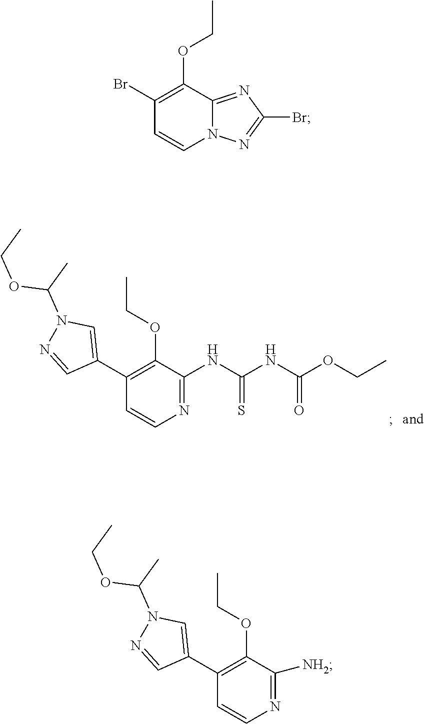 [SMILES: CCOc1c(Br)ccn2nc(Br)nc12 ; CCOC(=O)NC(=S)Nc1nccc(-c2cnn(C(C)OCC)c2)c1OCC ; CCOc1c(-c2cnn(C(C)OCC)c2)ccnc1N]
or a salt thereof.

69. A hemi-succinate salt of a compound of Formula (1a): [chemical structure]
